(19) United States
(12) Patent Application Publication
(10) Pub. No.: US 2026/0027116 A1
(43) Pub. Date: Jan. 29, 2026

(54) Methods of Treating Chronic Graft-Versus-Host Disease Following Allogeneic Hematopoietic Stem Cell Transplantation

(71) Applicant: Kadmon Corporation, LLC, Morristown, NJ (US)

(72) Inventors: Sanjay Aggarwal, Cambridge, MA (US); Mark Berger, Cambridge, MA (US); David Eiznhamer, Cambridge, MA (US); Jeegar P. Patel, Hillsborough, NJ (US); Olivier Schueller, Cambridge, MA (US)

(73) Assignee: Kadmon Corporation, LLC, Morristown, NJ (US)

(21) Appl. No.: 19/259,700

(22) Filed: Jul. 3, 2025

Related U.S. Application Data
(63) Continuation of application No. 18/302,483, filed on Apr. 18, 2023, now abandoned.
(60) Provisional application No. 63/332,632, filed on Apr. 19, 2022.

(51) Int. Cl. A61K 31/517; A61P 37/06

(52) U.S. Cl. CPC A61K 31/517; A61P 37/06

(57) ABSTRACT

The present disclosure relates generally to the treatment of patients with chronic graft-versus-host disease (cGVHD) following allogeneic hematopoietic stem cell transplantation by administering belumosudil at certain dosages.

### CROSS-REFERENCE TO RELATED APPLICATIONS

This application is a continuation of U.S. application Ser. No. 18/302,483, filed Apr. 18, 2023, which claims priority to U.S. Provisional Application No. 63/332,632, filed Apr. 19, 2022, the entire contents of which are incorporated by reference herein for all purposes.

### TECHNICAL FIELD

The present disclosure relates generally to the treatment of patients with chronic graft-versus-host disease (cGVHD) following allogeneic hematopoietic stem cell transplantation by administering belumosudil.

### BACKGROUND

Chronic graft-versus-host disease (cGVHD) is an immune-mediated inflammatory and fibrotic disorder. (MacDonald KPA, Hill G R, Blazar B R: Chronic graft-versus-host disease: Biological insights from preclinical and clinical studies. Blood 129:13-21, 2017) It is a leading cause of morbidity, mortality, and impaired quality of life (QOL) after an allogeneic hematopoietic cell transplant (alloHCT). (Arora M, Cutler C S, Jagasia M H, et al: Late acute and chronic graft-versus-host disease after allogeneic hematopoietic cell transplantation. Biol Blood Marrow Transpl 22:449-455, 2016; Wingard J R, Majhail N S, Brazauskas R, et al: Long-term survival and late deaths after allogeneic hematopoietic cell transplantation. J Clin Oncol 29:2230-2239, 2011; Wong F L, Francisco L, Togawa K, et al: Long-term recovery after hematopoietic cell transplantation: Predictors of quality-of-life concerns. Blood 115:2508-2519, 2010). cGVHD affects up to 70% of all alloHCT recipients, with an incidence of 20%-50% in children, who survive more than 100 days after alloHCT. (Arora M, Cutler C S, Jagasia M H, et al: Late acute and chronic graft-versus-host disease after allogeneic hematopoietic cell transplantation. Biol Blood Marrow Transpl 22:449-455, 2016; Baird K, Cooke K, Schultz K R: Chronic graft versus host disease (GVHD) in children. Pediatr Clin North Am 57:297-322, 2010; Lec S J, Vogelsang G, Flowers M E D: Chronic graft-versus-host disease. Biol Blood Marrow Transpl 9:215-233, 2003) It is the leading cause of nonrelapse mortality beyond 2 years after alloHCT. The estimated prevalence of cGVHD is 14,000 patients in the United States (as of 2016). (Bachier C R, Aggarwal S K, Hennegan K, et al: Epidemiology and real-world treatment of chronic graft-versus-host disease post allogeneic hematopoietic cell transplantation: A US claims analysis. Presented at ASH 2019, Orlando, FL, Dec. 7-10, 2019)

Of those patients, approximately 40% have severe disease. (Lee S J, Nguyen T D, Onstad L, et al: Success of immunosuppressive treatments in patients with chronic graft-versus-host disease. Biol Blood Marrow Transpl 24:555-562, 2018; Saillard C, Crocchiolo R, Furst S, et al: National Institutes of Health classification for chronic graft-versus-host disease predicts outcome of allo-hematopoietic stem cell transplant after fludarabine-busulfan-antithymocyte globulin conditioning regimen. Leuk Lymphoma 55:1106-1112, 2014) In addition, 42% of patients have ≥4 organs involved at the time of diagnosis. Patients with cGVHD have substantial impairment in QOL as assessed by the Lec Symptom Scale (LSS), which measures the effect of cGVHD on patients' functioning and well-being. (Pidala J, Kurland B, Chai X, ct al: Patient-reported quality of life is associated with severity of chronic graft-versus-host disease as measured by NIH criteria: Report on baseline data from the chronic GVHD Consortium. Blood 117:4651-4657, 2011; Lee S J, Cook E F, Soiffer R, et al: Development and validation of a scale to measure symptoms of chronic graft-versus-host disease. Biol Blood Marrow Transpl 8:444-452, 2002) Only one third of patients who have cGVHD and start systemic treatment will be alive, in remission and off immunosuppressive therapy by 5 years. (Lee S J, Nguyen T D, Onstad L, et al: Success of immunosuppressive treatments in patients with chronic graft-versus-host disease. Biol Blood Marrow Transpl 24:555-562, 2018)

The pathophysiology of cGVHD can be separated into three phases: early inflammation because of tissue injury, a dysregulated adaptive immune system, and chronic inflammation and aberrant tissue repair with fibrosis. (Zeiser R, Blazar B R: Pathophysiology of chronic graft-versus-host disease and therapeutic targets. N Engl J Med 377:2565-2579, 2017)

First-line therapy for National Institutes of Health (NIH)-defined moderate to severe chronic graft-versus-host disease (cGVHD) is corticosteroids alone or in combination with sirolimus or a calcincurin inhibitor. (Carpenter P A, Logan B R, Lee S J, et al: A phase II/III randomized, multicenter trial of prednisone/sirolimus versus prednisone/sirolimus/calcineurin inhibitor for the treatment of chronic graft-versus-host disease: BMT CTN 0801. Haematologica 103:1915-1924, 2018). However, up to 70% of patients require additional lines of therapy. (Bachier C R, Aggarwal S K, Hennegan K, et al: Epidemiology and real-world treatment of chronic graft-versus-host disease post allogeneic hematopoietic cell transplantation: A US claims analysis. Presented at ASH 2019, Orlando, FL, Dec. 7-10, 2019; Lee S J, Nguyen T D, Onstad L, et al: Success of immunosuppressive treatments in patients with chronic graft-versus-host disease. Biol Blood Marrow Transpl 24:555-562, 2018: Flowers M E D, Martin P J: How we treat chronic graft-versus-host disease. Blood 125:606-615, 2015). Furthermore, the long-term use of corticosteroids is associated with significant side effects. (Biol Blood Marrow Transpl 24:555-562, 2018: Flowers M E D, Martin P J: How we treat chronic graft-versus-host disease. Blood 125:606-615, 2015); MacDonald KPA, Hill G R, Blazar B R: Chronic graft-versus-host disease: Biological insights from preclinical and clinical studies. Blood 129:13-21, 2017)

Management of cGVHD continues to evolve with the advent of targeted therapies. In 2017, the US Food and Drug Administration approved ibrutinib, a Bruton's Tyr kinase inhibitor, for the treatment of adults with cGVHD after failure of ≥1 systemic lines of therapy. (Imbruvica. Package Insert. Sunnyvale, CA, Pharmacyclics LLC, 2019) In patients with cGVHD who were required to have either >25% body surface area erythematous rash or an NIH mouth score of >4, (Miklos D, Cutler C S, Arora M, et al: Ibrutinib for chronic graft-versus-host disease after failure of prior therapy. Blood 130:2243-2250, 2017) a study with ibrutinib reported an overall response rate (ORR) of 67% and a discontinuation rate because of treatment-emergent adverse events (TEAEs) of 43%. (Waller E K, Miklos D, Cutler C, et al: Ibrutinib for chronic graft-versus-host disease after failure of prior therapy: 1-Year update of a phase 1b/2 study. Biol Blood Marrow Transpl 25:2002-2007, 2019)

There remains an opportunity to study other treatment options for patients who have failed ≥1 lines of therapy.

### SUMMARY

The present disclosure provides methods of treating a subject diagnosed with chronic graft-versus-host disease following allogeneic hematopoietic stem cell transplantation by administering a therapeutically effective amount of 2-{3-[4-(1H-indazol-5-ylamino)-2-quinazolinyl]phenoxy}-N-(propan-2-yl) acetamide, or a pharmaceutically acceptable salt thereof (belumosudil) to a subject in need thereof. In some embodiments, belumosudil can be administered to the subject at a dose selected from the group consisting of 200 mg daily, 200 mg twice daily, and 400 mg daily. In some embodiments, belumosudil can be administered to the subject at a dose selected from the group consisting of 200 mg daily, 200 mg twice daily, and 400 mg daily, wherein the belumosudil is administered as a 28-day cycle, wherein the number of cycles ranges from 3-15.

The present embodiments can be understood more fully by reference to the detailed description and examples, which are intended to exemplify non-limiting embodiments.

### BRIEF DESCRIPTION OF THE DRAWINGS

FIG. 1 is the CONSORT flow diagram describing the phase IIa, open-label, dose-finding study of belumosudil of Example 1.

FIG. 2 is a forest plot for subgroup analyses of ORR in the safety population. Subgroups were defined based on baseline assessment.

FIG. 3A describes the best individual response by organ system among responders. n=number of responder population for global severity rating and number of specific organs involved at baseline. The percentages are calculated based on the corresponding n.

FIG. 3B is a response and progression heat map for all patients in the safety population. Of 11 patients with progression in joints, seven had a reduction in P-ROM of just one unit.

FIG. 4 describes time to response among belumosudil responders. Percentages are calculated based on the number of responder population.

FIG. 5 describes time to response by selected organs among responders. Percentages are calculated based on the number of responder population.

FIG. 6A describes changes in percentage of CD41 Tregs following treatment with belumosudil compared with baseline for Tregs (regulatory T cells all). Predose peripheral blood samples were collected on C1D1 (cycle 1 day 1), C2D1 (cycle 2 day 1), C4D1 (cycle 4 day 1), C7D1 (cycle 7 day 1), and end-of-treatment visits.

FIG. 6B describes changes in percentage of CD41 Tregs following treatment with belumosudil compared with baseline for Tregs (regulatory T cells responders). Predose peripheral blood samples were collected on C1D1 (cycle 1 day 1), C2D1 (cycle 2 day 1), C4D1 (cycle 4 day 1), C7D1 (cycle 7 day 1), and end-of-treatment visits.

FIG. 6C describes changes in percentage of CD41 Tregs following treatment with belumosudil compared with baseline for Tregs (regulatory T cells nonresponders). Predose peripheral blood samples were collected on C1D1 (cycle 1 day 1), C2D1 (cycle 2 day 1), C4D1 (cycle 4 day 1), C7D1 (cycle 7 day 1), and end-of-treatment visits.

FIG. 7 is the CONSORT flow diagram describing the phase II randomized study of belumosudil of Example 2.

FIG. 8 is a forest plot of subgroup analyses of ORR (mITT). High ORRs were observed in all subgroups analyzed in the mITT population, and efficacy was maintained irrespective of prior treatments. The 50th percentile for duration of cGVHD before enrollment was 29 months. Response assessments performed on or after the initiation of a new systemic therapy for cGVHD were excluded from the analysis.

FIG. 9 describes ORR by organ system in the mITT population. Organ-specific analyses in the mITT population demonstrated ORRs in the skin, eyes, mouth, liver, lungs, joints/fascia, upper GI tract, lower GI tract, and esophagus. CR was seen across all affected organs.

FIG. 10A describes durability of response to belumosudil by dose. Kaplan-Meier plot of DOR in the responder population. DOR was defined as the time from response until documented progression or start of another cGVHD systemic treatment; durability data continue to mature.

FIG. 10B describes durability of response to belumosudil by dose. Kaplan-Meier curves of estimated FFS in the mITT population, including reasons for failure. FFS was defined as the absence of cGVHD treatment change, NRM, and recurrent malignancy.

FIG. 10C describes durability of response to belumosudil by dose. Kaplan-Meier curves of estimated OS in the mITT population.

FIG. 11 describes the clinical study design of Example 2.

### DETAILED DESCRIPTION

### Definitions

The terms “allogeneic hematopoietic stem cell transplantation (allo-HSCT)” also called bone marrow transplantation or stem cell transplantation, or “allogeneic hematopoietic cell transplantation (allo-HCT)” in which the hematopoietic cells from a donor are grafted into a recipient who is not an identical twin. The source of hematopoietic stem cells for allogeneic transplantation may be peripheral blood stem cells (PBSC) or bone marrow (BM). In some circumstances umbilical cord blood may be used. The donor and recipient may be matched at the human leukocyte antigen (HLA) genes, such as siblings. The donor and recipient may be a parent and a child who are only half-matched (haploidentical). A myeloablative transplant uses very high doses of chemotherapy or radiation prior to transplantation with autologous or allogeneic hematopoietic stem cells. A non-myeloablative transplant, or reduced intensity transplant, allows the patient to have less intensive chemotherapy before transplantation with allogeneic hematopoietic stem cells.

Belumosudil is an oral selective rho-associated coiled-coil-containing protein kinase-2 (ROCK2) inhibitor. ROCK2 inhibition acts on the dysregulated adaptive immune system and the fibrosis that occurs as a result of aberrant tissue repair. (Zanin-Zhorov A, Weiss J M, Nyuydzefe M S, Chen W, Scher J U, Mo R et al. Selective oral ROCK2 inhibitor down-regulates IL-21 and IL-17 secretion in human T cells via STAT3-dependent mechanism. Proceedings of the National Academy of Sciences of the United States of America 2014; 111 (47): 16814-16819. Flynn R, Paz K, Du J, Reichenbach D K, Taylor P A, Panoskaltsis-Mortari A et al. Targeted Rho-associated kinase 2 inhibition suppresses murine and human chronic GVHD through a Stat3-dependent mechanism. Blood 2016; 127 (17): 2144-2154.)

The term “belumosudil” as used herein is intended to include 2-{3-[4-(1H-indazol-5-ylamino)-2-quinazolinyl]phenoxy}-N-(propan-2-yl) acetamide, and any pharmaceutically acceptable salt thereof. Belumosudil, also known as KD025, is marketed as REZUROCK™ in the United States for the treatment of patients with chronic GVHD after failure of at least two prior lines of systemic therapy. The active pharmaceutical ingredient of REZUROCK™ is belumosudil mesylate with the molecular formula C27H28N605S and the molecular weight is 548.62 g/mol. The chemical name for belumosudil mesylate is 2-{3-[4-(1H-indazol-5-ylamino)-2-quinazolinyl]phenoxy}-N-(propan-2-yl) acetamide methanesulfonate (1:1). The chemical structure of belumosudil mesylate is as follows:

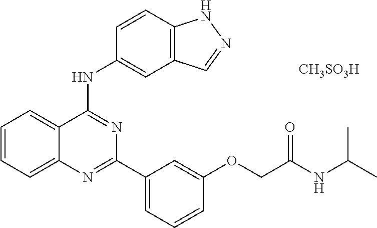

Belumosudil mesylate is a yellow powder that is practically insoluble in water, slightly soluble in methanol and DMF and soluble in DMSO. Belumosudil tablets are for oral administration. Each tablet contains 200 mg of the free base equivalent to 242.5 mg of belumosudil mesylate. The tablet also contains the following inactive ingredients: microcrystalline cellulose, hypromellose, croscarmellose sodium, colloidal silicon dioxide, and magnesium stearate. The tablet film consists of polyvinyl alcohol, polyethylene glycol, talc, titanium dioxide and yellow iron oxide. Each 200 mg tablet is a pale yellow film-coated oblong tablet debossed with “KDM” on one side and “200” on the other side. Tablets are stored at room temperature, 20° C. to 25° C. (68° F. to 77° F.); excursions permitted from 15° C. and 30° C. (59° F. to 86° F.).

Belumosudil is described in the following US patents: U.S. Pat. Nos. 8,357,693, 9,815,820, 10,183,931, and 10,696,660.

Belumosudil is an inhibitor of rho-associated, coiled-coil containing protein kinase (ROCK) which inhibits ROCK2 and ROCK1 with IC50 values of approximately 100 nM and 3 μM, respectively. Belumosudil down-regulated proinflammatory responses via regulation of STAT3/STAT5 phosphorylation and shifting Th17/Treg balance in ex-vivo or in vitro-human T cell assays. Belumosudil also inhibited aberrant pro-fibrotic signaling, in vitro. In vivo, belumosudil demonstrated activity in animal models of chronic GVHD.

First-line therapy for National Institutes of Health (NIH)-defined moderate to severe chronic graft-versus-host disease (cGVHD) is corticosteroids alone or in combination with sirolimus or a calcineurin inhibitor. (Carpenter P A, Logan B R, Lec S J, et al: A phase II/III randomized, multicenter trial of prednisone/sirolimus versus prednisone/sirolimus/calcineurin inhibitor for the treatment of chronic graft-versus-host disease: BMT CTN 0801. Haematologica 103:1915-1924, 2018). Examples of corticosteroid therapies for treatment of cGVHD include, but are not limited to, prednisone, prednisolone, methylprednisolone, budesonide.

Lee Symptom Scale (LSS) summary score measures the effect on patients' functioning and well-being. The Lee Symptom Scale is a 30-item scale developed to measure the symptoms of cGVHD and is described in Lec S J, Cook E F, Soiffer R, Antin J H. Development and validation of a scale to measure symptoms of chronic graft-versushost disease. Biol Blood Marrow Transplant 2002; 8:444-452.

As used herein, the term “line of treatment” or “line of therapy” describes the sequence or order in which different therapies are given to a patient as the patient's disease progresses. Initial treatment (first-line therapy) may not work or may stop working after a period of time. After such first-line therapy is discontinued, a second different treatment (second-line therapy) may be given. Subsequent lines of therapy may be given when a second-line therapy does not work or stops working. Some patients may be administered multiple lines of therapy over the course of a disease. Examples of prior systemic therapies for treating cGVHD include, but are not limited to, prednisone, tacrolimus, ECP, sirolimus, ibruitinib, ruxolitinib, MMF, rituximab, MTX, cyclosporine, imatinib, ixazomib, and ofatumumab.

The term “subject” or “patient” as used herein includes an animal or a human.

The term “NIH lung symptom score” or “NIH cGVHD lung score” is a clinical symptom-based score ranging from 0 to 3. A Score 0 is used for no symptoms, Score 1 is used for symptoms of shortness of breath with stairs, Score 2 is used for symptoms of shortness of breath on flat ground, and Score 3 is used for shortness of breath at rest or requiring oxygen.

Examples of clinical endpoints include the following. Overall response rate (ORR) is the percentage of people in a study or treatment group who have a partial response (PR) or complete response (CR) to the treatment within a certain period of time. Failure-free survival (FFS) means the time from the first dose of belumosudil to a failure event, or the interval between the start of belumosudil and the addition of a new cGVHD therapy, relapse of the underlying disease, or nonrelapse mortality (NRM). Overall survival (OS) means the length of time from either the date of diagnosis or the start of treatment for a disease. Duration of response (DOR) means from the time of initial response (e.g., PR or CR) until documented progression from best response of cGVHD, time from initial response to start of additional systemic cGVHD therapy, or death. Time to next treatment (TTNT) means time to initiation of a subsequent systemic cGVHD therapy.

Steroid-refractory (SR) cGVHD is defined as cGVHD progression while on prednisone at ≥1 mg/kg/day for 1-2 weeks, or stable cGVHD while on ≥0.5 mg/kg/day for 1-2 months.

Immunosuppressive therapy (IST) is typically administered for at least six months after allo-HSCT in order to prevent GVHD. Examples of IST's include sirolimus, prednisone and calcineurin inhibitors such as tacrolimus and cyclosporine. In some embodiments, standard-of-care immunosuppressants may comprise at least one of the following: calcineurin inhibitor, a cell cycle inhibitor and an mTOR inhibitor.

In some embodiments, provided herein are methods of treating chronic graft-versus-host disease following allogeneic hematopoietic stem cell transplantation, the methods comprising administering belumosudil at a dose selected from the group consisting of 200 mg daily, 200 mg twice daily, and 400 mg daily to a subject in need thereof.

In some embodiments, provided herein are methods of treating chronic graft-versus-host disease following allogeneic hematopoietic stem cell transplantation, the method comprising administering belumosudil to a subject in need thereof at a dose selected from the group consisting of 200 mg daily, 200 mg twice daily, and 400 mg daily, wherein the belumosudil is administered as a 28-day cycle, wherein the number of cycles ranges from 3 to 15.

In some embodiments, the number of cycles ranges from 3 cycles to loss of response. In some embodiments, the number of cycles ranges from 4 cycles to loss of response. In some embodiments, the number of cycles ranges from 5 cycles to loss of response. In some embodiments, the number of cycles ranges from 6 cycles to loss of response. In some embodiments, the number of cycles ranges from 7 cycles to loss of response. In some embodiments, the number of cycles ranges from 8 cycles to loss of response. In some embodiments, the number of cycles is greater than 3, 4, 5, 10, 15, 20, 25, or 30, or until a desired response is achieved. In some embodiments, the belumosudil is administered until a desired response is achieved. In some embodiments, a desired response comprises no further disease progression. In some embodiments, a desired response comprises slowing the disease progression. In some embodiments, a desired response comprises a reduction in the Lee Symptom Scale Score. In some embodiments, a desired response comprises at least a 10-point reduction in the Lee Symptom Scale Score. In some embodiments, the belumosudil is administered until there is no disease progression. In some embodiments, the belumosudil is administered until there is at least a 10-point reduction in the Lee Symptom Scale Score. In some embodiments, the subject is human.

In some embodiments, use of a therapeutically effective amount of 2-{3-[4-(1H-indazol-5-ylamino)-2-quinazolinyl]phenoxy}-N-(propan-2-yl) acetamide (belumosudil), or a pharmaceutically acceptable salt thereof for preparing a medicament for the treatment of chronic graft-versus-host disease following allogeneic hematopoietic stem cell transplantation is provided.

In some embodiments, a compound comprising a therapeutically effective amount of 2-{3-[4-(1H-indazol-5-ylamino)-2-quinazolinyl]phenoxy}-N-(propan-2-yl) acetamide (belumosudil), or a pharmaceutically acceptable salt thereof for use in the treatment of chronic graft-versus-host disease following allogeneic hematopoietic stem cell transplantation is provided.

In some embodiments, use of a dose selected from the group consisting of 200 mg daily, 200 mg twice daily, and 400 mg daily of 2-{3-[4-(1H-indazol-5-ylamino)-2-quinazolinyl]phenoxy}-N-(propan-2-yl) acetamide (belumosudil), or a pharmaceutically acceptable salt thereof for preparing a medicament for the treatment of chronic graft-versus-host disease following allogeneic hematopoietic stem cell transplantation is provided.

In some embodiments, a compound comprising a dose selected from the group consisting of 200 mg daily, 200 mg twice daily, and 400 mg daily of 2-{3-[4-(1H-indazol-5-ylamino)-2-quinazolinyl]phenoxy}-N-(propan-2-yl) acetamide (belumosudil), or a pharmaceutically acceptable salt thereof for use in the treatment of chronic graft-versus-host disease following allogeneic hematopoietic stem cell transplantation is provided.

In some embodiments, use of a dose selected from the group consisting of 200 mg daily, 200 mg twice daily, and 400 mg daily of 2-{3-[4-(1H-indazol-5-ylamino)-2-quinazolinyl]phenoxy}-N-(propan-2-yl) acetamide (belumosudil), or a pharmaceutically acceptable salt thereof, wherein the belumosudil is administered as a 28-day cycle, wherein the number of cycles ranges from 3 to 15, for preparing a medicament for the treatment of chronic graft-versus-host disease following allogeneic hematopoietic stem cell transplantation is provided.

In some embodiments, a compound comprising a dose selected from the group consisting of 200 mg daily, 200 mg twice daily, and 400 mg daily of 2-{3-[4-(1H-indazol-5-ylamino)-2-quinazolinyl]phenoxy}-N-(propan-2-yl) acetamide (belumosudil), or a pharmaceutically acceptable salt thereof, wherein the belumosudil is administered as a 28-day cycle, wherein the number of cycles ranges from 3 to 15, for use in the treatment of chronic graft-versus-host disease following allogeneic hematopoietic stem cell transplantation is provided.

In some embodiments, the allogeneic hematopoietic stem cell transplantation is a matched-HSCT. In some embodiments, the allogeneic hematopoietic stem cell transplantation is a haploidentical-HSCT.

In some embodiments, the belumosudil is administered in a 28-day cycle.

In some embodiments, the number of cycles ranges from 3 to 15. In some embodiments, the number of cycles ranges from 3 to 14, from 3 to 13, from 3 to 12, from 3 to 11, from 3 to 10, from 3 to 9, from 3 to 8, from 3 to 7, from 3 to 6, from 3 to 5, or from 3 to 4. In some embodiments, the number of cycles ranges from 5 to 11. In some embodiments, the number of cycles ranges from 6 to 12. In some embodiments, the number of cycles ranges from 5 to 10, from 5 to 9, or from 5 to 8. In some embodiments, the number of cycles ranges from 5 to 7. In some embodiments, the number of cycles ranges from 5 to 6. In some embodiments, the number of cycles is 5. In some embodiments, the number of cycles is 6. In some embodiments, the number of cycles is 7. In some embodiments, the number of cycles is 3, 4, 5, 6, 7, 8, 9, 10, 11, 12, 13, 14, or 15.

In some embodiments, the subject experiences an improvement as defined by the Lee Symptom Scale (LSS). In some embodiments, the subject experiences at least a 7-point reduction in the LSS score. In some embodiments, the subject experiences at least a 10-point reduction in the LSS score. In some embodiments, the improvement is maintained over at least two consecutive evaluations. In some embodiments the LSS score is evaluated at baseline and on day 1 of each cycle starting at cycle 2 day 1.

In some embodiments, the belumosudil is administered to the subject at a dose selected from the group consisting of 200 mg daily, 200 mg twice daily, and 400 mg daily. In some embodiments, the dose is 200 mg daily. In some embodiments, the dose is 200 mg twice daily. In some embodiments, the dose is 400 mg daily.

In some embodiments, the subject has chronic graft-versus-host disease and has failed one to three prior lines of systemic therapy for the chronic graft-versus-host disease. In some embodiments, the subject has chronic graft-versus-host disease and has failed at least two prior lines of systemic therapy for the chronic graft-versus-host disease. In some embodiments, the subject has chronic graft-versus-host disease and has failed two to five prior lines of systemic therapy for the chronic graft-versus-host disease. In some embodiments, the subject has failed at least one, at least two, at least three, at least four, at least five, at least six, at least seven, at least eight, at least nine or at least ten prior lines of systemic therapy for the chronic graft-versus-host disease.

In some embodiments, the subject experienced a complete response to last treatment for the graft-versus-host disease prior to belumosudil. In some embodiments, the subject experienced a partial response to last treatment for the graft-versus-host disease prior to belumosudil. In some embodiments, the subject experienced stable disease during the last treatment for the graft-versus-host disease prior to belumosudil.

In some embodiments, the prior lines of systemic therapy for the chronic graft-versus-host disease have been discontinued.

In some embodiments, the prior lines of systemic therapy are selected from the group consisting of prednisone, tacrolimus, ECP, sirolimus, ibruitinib, ruxolitinib, MMF, rituximab, MTX, cyclosporine, imatinib, ixazomib, and ofatumumab.

In some embodiments, the cGVHD is steroid-refractory (SR) cGVHD. In some embodiments, the subject is refractory to the last line of treatment prior to belumosudil treatment.

In some embodiments, the subject is receiving concomitant corticosteroid therapy. In some embodiments, the concomitant corticosteroid therapy is selected from the group consisting of prednisone, prednisolone, methylprednisolone, and budesonide. In some embodiments, the concomitant corticosteroid therapy is prednisone. In some embodiments, the dose of the concomitant corticosteroid therapy is reduced after at least 1 cycle of the belumosudil treatment. In some embodiments, the dose of the concomitant corticosteroid therapy is reduced by at least about 10%, by at least about 20%, by at least about 30%, by at least about 40%, by at least about 50%, by at least about 60%, or by at least about 70% after at least 1 cycle of the belumosudil treatment. In some embodiments, the dose of the concomitant corticosteroid therapy is reduced by from about 10% to about 70%, from about 15% to about 65%, from about 20% to about 60%, from about 30% to about 60%, from about 35% to about 60%, from about 40% to about 60%, or from about 45% to about 55% after at least 1 cycle of the belumosudil treatment. In some embodiments, the concomitant corticosteroid therapy is discontinued after at least 1 cycle of the belumosudil treatment.

In some embodiments, the subject is receiving concomitant calcineurin inhibitor therapy. In some embodiments, the subject is receiving at least one of the following: calcineurin inhibitor, a cell cycle inhibitor and an mTOR inhibitor.

In some embodiments, the subject has involvement of at least 4 organs. In some embodiments, the subject has involvement of at least 3 organs. In some embodiments, the subject has involvement of at least 2 organs. In some embodiments, the subject has involvement of one or more; of two or more; of three or more; or of four or more, of the following organs: skin, joints/fascia, eyes, mouth, lungs, esophagus, upper gastrointestinal tract, lower gastrointestinal tract, and liver.

In some embodiments, the subject has received an allogenic bone marrow transplant or hematopoietic stem cell transplantation. In some embodiments, the subject is receiving glucocorticoid therapy and calcineurin therapy. In some embodiments, the subject is receiving glucocorticoid therapy. In some embodiments, the subject is receiving one or more concomitant therapies that are not generally considered to be immunosuppressive. In some embodiments, the subject is receiving concomitant extracorporeal photopheresis (ECP). In some embodiments, the subject has persistent active cGVHD manifestations, as defined by the 2014 NIH Consensus Development Project on Criteria for Clinical Trials in cGVHD, after at least 2 months of corticosteroid therapy. In some embodiments, the subject has received no more than 3 prior lines of treatment for cGVHD. In some embodiments, the subject has a Karnofsky Performance Scale of >40. In some embodiments, the subject has an absolute neutrophil count ≥1.5×10⁹/L (without myeloid growth factors in the previous week) and a platelet count≥50×10⁹/L (without transfusion or thrombopoietin or thrombopoietin analogues within the previous 2 weeks). In some embodiments, the subject has total bilirubin≤1.5× upper limit of normal (ULN), ALT and AST≤3×ULN, and a glomerular filtration rate (GFR)≥30 mL/min/1.73 m² using the 4-Variable Modification of Diet in Renal Disease (MDRD-4) variable formula. In some embodiments, the subject is not pregnant. In some embodiments, the subject is at least 18 years old.

In some embodiments, the subject has at least one of the following characteristics: has received an allogenic bone marrow transplant; has received a hematopoietic stem cell transplantation; is receiving glucocorticoid therapy and calcineurin therapy; is receiving glucocorticoid therapy; is receiving one or more concomitant therapies that are not generally considered to be immunosuppressive; is receiving concomitant extracorporeal photopheresis [ECP]; has persistent active cGVHD manifestations as defined by the 2014 NIH Consensus Development Project on Criteria for Clinical Trials in cGVHD, after at least 2 months of corticosteroid therapy; has received no more than 3 prior lines of treatment for cGVHD; has a Karnofsky Performance Scale of >40; has an absolute neutrophil count≥1.5×10⁹/L (without myeloid growth factors within the previous week) and a platelet count≥50×10⁹/L (without transfusion or thrombopoietin or thrombopoietin analogues within the previous 2 weeks); has total bilirubin≤1.5× upper limit of normal (ULN), ALT and AST≤3×ULN, and a glomerular filtration rate (GFR)≥30 mL/min/1.73 m² using the 4-Variable Modification of Diet in Renal Disease (MDRD-4) variable formula; is not pregnant.

In some embodiments, the subject is not concomitantly receiving an investigational GVHD treatment. In some embodiments, the subject does not have acute GVHD. In some embodiments, the subject is not pregnant or breastfeeding. In some embodiments, the subject is not taking any medication generally known to be a moderate or strong inhibitor of the CYP3A4 isozyme or any drugs that are moderate or strong CYP3A4 inducers. In some embodiments, the subject does not have a history of poorly controlled psychiatric disease. In some embodiments, the subject does not have a history of coronary artery disease. In some embodiments, the subject does not have regular and excessive use of alcohol in the previous 6 months, defined as alcohol intake >14 drinks per week in a male or >7 drinks per week in a female (where approximately 10 g of alcohol equals one “drink” unit, and one unit equals 1 ounce of distilled spirits, one 12-ounce beer, or one 4-ounce glass of wine). In some embodiments, the subject does not have a history of human immunodeficiency virus (HIV) or active hepatitis C virus (HCV) or hepatitis B virus (HBV). In some embodiments, the subject is not diagnosed with another malignancy (other than malignancy for which transplant was performed) within the previous 3 years, with the exception of completely resected basal cell or squamous cell carcinoma of the skin, resected in situ cervical malignancy, resected breast ductal carcinoma in situ, or low-risk prostate cancer after curative resection. In some embodiments, the subject does not have relapse of the underlying cancer or post-transplant lymphoproliferative disease. In some embodiments, the subject does not have previous exposure to belumosudil or known allergy/sensitivity to belumosudil or any other ROCK2 inhibitor. In some embodiments, the subject is not taking other immunosuppressant drugs for GVHD, including mTOR (mammalian target of rapamycin) inhibitors. In some embodiments, the subject does not have a QTcF>450 msec.

In some embodiments, the subject has at least one of the following characteristics: is not concomitantly receiving an investigational GVHD treatment; does not have acute GVHD; is not pregnant or breastfeeding; is not taking any medication generally known to be a moderate or strong inhibitor of the CYP3A4 isozyme or any drugs that are moderate or strong CYP3A4 inducers; does not have a history of poorly controlled psychiatric disease; does not have a history of coronary artery disease; does not have regular and excessive use of alcohol in the previous 6 months, defined as alcohol intake>14 drinks per week in a male or >7 drinks per week in a female (where approximately 10 g of alcohol equals one “drink” unit, and one unit equals 1 ounce of distilled spirits, one 12-ounce beer, or one 4-ounce glass of wine); does not have a history of human immunodeficiency virus (HIV); does not have a history of active hepatitis C virus (HCV); does not have a history of hepatitis B virus (HBV); is not diagnosed with another malignancy (other than malignancy for which transplant was performed) within the previous 3 years, with the exception of completely resected basal cell or squamous cell carcinoma of the skin, resected in situ cervical malignancy, resected breast ductal carcinoma in situ, or low-risk prostate cancer after curative resection; does not have relapse of the underlying cancer or post-transplant lymphoproliferative disease; does not have previous exposure to belumosudil; does not have a known allergy/sensitivity to belumosudil or any other ROCK2 inhibitor; is not taking other immunosuppressant drugs for GVHD, including mTOR (mammalian target of rapamycin) inhibitors; and does not have a QTcF>450 msec.

In some embodiments, the subject has at least one of the following characteristics: has received an allogenic bone marrow transplant; has received a hematopoietic stem cell transplantation; is receiving glucocorticoid therapy and calcineurin therapy; is receiving glucocorticoid therapy; is receiving one or more concomitant therapies that are not generally considered to be immunosuppressive; is receiving concomitant extracorporcal photopheresis [ECP]; has persistent active cGVHD manifestations as defined by the 2014 NIH Consensus Development Project on Criteria for Clinical Trials in cGVHD, after at least 2 months of corticosteroid therapy; has received no more than 3 prior lines of treatment for cGVHD; has a Karnofsky Performance Scale of >40; has an absolute neutrophil count≥1.5×10⁹/L (without myeloid growth factors within the previous week) and a platelet count≥50×10⁹/L (without transfusion or thrombopoietin or thrombopoietin analogues within the previous 2 weeks); has total bilirubin≤1.5× upper limit of normal (ULN), ALT and AST≤3×ULN, and a glomerular filtration rate (GFR)≥30 mL/min/1.73 m² using the 4-Variable Modification of Diet in Renal Disease (MDRD-4) variable formula; is not pregnant or breastfeeding; is not concomitantly receiving an investigational GVHD treatment; does not have acute GVHD; is not taking any medication generally known to be a moderate or strong inhibitor of the CYP3A4 isozyme or any drugs that are moderate or strong CYP3A4 inducers; does not have a history of poorly controlled psychiatric disease; does not have a history of coronary artery disease; does not have regular and excessive use of alcohol in the previous 6 months, defined as alcohol intake>14 drinks per week in a male or >7 drinks per week in a female (where approximately 10 g of alcohol equals one “drink” unit, and one unit equals 1 ounce of distilled spirits, one 12-ounce beer, or one 4-ounce glass of wine); does not have a history of human immunodeficiency virus (HIV); does not have a history of active hepatitis C virus (HCV); does not have a history of hepatitis B virus (HBV); is not diagnosed with another malignancy (other than malignancy for which transplant was performed) within the previous 3 years, with the exception of completely resected basal cell or squamous cell carcinoma of the skin, resected in situ cervical malignancy, resected breast ductal carcinoma in situ, or low-risk prostate cancer after curative resection; does not have relapse of the underlying cancer or post-transplant lymphoproliferative disease; does not have previous exposure to belumosudil; does not have a known allergy/sensitivity to belumosudil or any other ROCK2 inhibitor; is not taking other immunosuppressant drugs for GVHD, including mTOR (mammalian target of rapamycin) inhibitors; and does not have a QTcF>450 msec.

In some embodiments, the subject has received an allogeneic hematopoietic cell transplant. In some embodiments, the subject has previously received at least 2 and not more than 5 lines of systemic therapy for cGVHD. In some embodiments, the subject has received glucocorticoid therapy with a stable dose over the previous 2 weeks. In some embodiments, the subject has persistent cGVHD manifestations. In some embodiments, the subject has a Karnofsky (if aged≥16 years)/Lansky (if aged <16 years) Performance Score of ≥60. In some embodiments, the subject has an absolute neutrophil count≥1.5×10⁹/L and a platelet count≥50×10⁹/L. In some embodiments, the subject has ALT and AST≤3×ULN, total bilirubin≤ 1.5×ULN, and a glomerular filtration rate (GFR)>30 mL/min/1.73 m² using the MDRD-4 variable formula. In some embodiments, the subject has a weight≥40 kg. In some embodiments, the subject is receiving concomitant corticosteroid therapy. In some embodiments, the subject is receiving concomitant calcineurin inhibitor therapy. In some embodiments, the subject is concomitantly receiving one or more of sirolimus, MMF, methotrexate, rituximab, and extracorporcal photopheresis (ECP) therapies.

In some embodiments, the subject has at least one of the following characteristics: has received an allogeneic hematopoietic cell transplant; has previously received at least 2 and not more than 5 lines of systemic therapy for cGVHD; has received glucocorticoid therapy with a stable dose over the previous 2 weeks; has persistent cGVHD manifestations; has a Karnofsky (if aged≥16 years)/Lansky (if aged <16 years) Performance Score of ≥60; has an absolute neutrophil count≥1.5×10⁹/L and a platelet count≥50×10⁹/L; has ALT and AST≤3×ULN, total bilirubin≤1.5×ULN, and a glomerular filtration rate (GFR)≥30 mL/min/1.73 m² using the MDRD-4 variable formula; has a weight≥40 kg; is receiving concomitant corticosteroid therapy; is receiving concomitant calcineurin inhibitor therapy; and is concomitantly receiving one or more of sirolimus, MMF, methotrexate, rituximab, and extracorporcal photophoresis (ECP) therapies.

In some embodiments, the subject is not receiving systemic cGVHD treatments. In some embodiments, the subject does not have histological relapse of the underlying cancer or post-transplant lymphoproliferative disease. In some embodiments, the subject is not receiving concomitant treatment with ibrutinib. In some embodiments, the subject does not have a history of human immunodeficiency virus (HIV) or hepatitis C virus (HCV) or a history of hepatitis B virus (HBV). In some embodiments, the subject is not diagnosed with another malignancy (other than malignancy for which transplant was performed) within the previous 3 years, with the exception of completely resected basal cell or squamous cell carcinoma of the skin, carcinoma in situ of the cervix, resected breast ductal carcinoma in situ, or prostate cancer with Gleason score <6 and stable PSA over 12 months. In some embodiments, the subject does not have previous exposure to belumosudil. In some embodiments, the subject does not have a known allergy/sensitivity to belumosudil or any other ROCK2 inhibitor. In some embodiments, the subject does not have a QTc (F)>480 msec. In some embodiments, the subject does not have a FEV1≤39% or a lung score of 3.

In some embodiments, the subject has at least one of the following characteristics: is not receiving systemic cGVHD treatments; does not have histological relapse of the underlying cancer or post-transplant lymphoproliferative disease; is not receiving concomitant treatment with ibrutinib; does not have a history of human immunodeficiency virus (HIV); does not have hepatitis C virus (HCV); does not have a history of hepatitis B virus (HBV); is not diagnosed with another malignancy (other than malignancy for which transplant was performed) within the previous 3 years, with the exception of completely resected basal cell or squamous cell carcinoma of the skin, carcinoma in situ of the cervix, resected breast ductal carcinoma in situ, or prostate cancer with Gleason score <6 and stable PSA over 12 months; does not have previous exposure to belumosudil; does not have a known allergy/sensitivity to belumosudil or any other ROCK2 inhibitor; does not have a QTc (F)>480 msec; and does not have a FEV1≤39% or a lung score of 3.

In some embodiments, the subject has at least one of the following characteristics: has received an allogeneic hematopoietic cell transplant; has previously received at least 2 and not more than 5 lines of systemic therapy for cGVHD; has received glucocorticoid therapy with a stable dose over the previous 2 weeks; has persistent cGVHD manifestations; has a Karnofsky (if aged≥16 years)/Lansky (if aged <16 years) Performance Score of ≥60; has an absolute neutrophil count≥1.5×10⁹/L and a platelet count≥50×10⁹/L; has ALT and AST≤3×ULN, total bilirubin≤1.5×ULN, and a glomerular filtration rate (GFR)≥30 mL/min/1.73 m² using the MDRD-4 variable formula; has a weight≥40 kg; is receiving concomitant corticosteroid therapy; is receiving concomitant calcineurin inhibitor therapy; is concomitantly receiving one or more of sirolimus, MMF, methotrexate, rituximab, and extracorporeal photopheresis (ECP) therapies; is at least 18 years old; is not receiving systemic cGVHD treatments; does not have histological relapse of the underlying cancer or post-transplant lymphoproliferative disease; is not receiving concomitant treatment with ibrutinib; does not have a history of human immunodeficiency virus (HIV); does not have hepatitis C virus (HCV); does not have a history of hepatitis B virus (HBV); is not diagnosed with another malignancy (other than malignancy for which transplant was performed) within the previous 3 years, with the exception of completely resected basal cell or squamous cell carcinoma of the skin, carcinoma in situ of the cervix, resected breast ductal carcinoma in situ, or prostate cancer with Gleason score <6 and stable PSA over 12 months; does not have previous exposure to belumosudil; does not have a known allergy/sensitivity to belumosudil or any other ROCK2 inhibitor; does not have a QTc (F)>480 msec; and does not have a FEV1≤39% or a lung score of 3.

In some embodiments, standard-of-care immunosuppressants may comprise at least one of the following: calcineurin inhibitor, a cell cycle inhibitor and an mTOR inhibitor.

### EXAMPLES

### Example 1: a Phase IIa, Open-Label, Dose-Finding Study of Belumosudil

### Subject Eligibility

Eligible patients were allogeneic bone marrow transplant or allogeneic hematopoietic cell transplant (alloHCT) recipients of age≥18 years with persistent cGVHD manifestations after having received one to three prior systemic lines of therapy and who were receiving corticosteroid treatment with or without a calcincurin inhibitor and/or concurrent extracorporeal photopheresis. Belumosudil was continued until cGVHD progression or unacceptable toxicity.

Inclusion criteria. Adult male and female subjects at least 18 years of age who had allogenic bone marrow transplant or hematopoietic stem cell transplantation. Received glucocorticoid therapy and calcineurin therapy or glucocorticoid therapy alone for cGVHD at study entry. Participants on calcineurin therapy only, without glucocorticoid therapy, were not eligible. Participants also received other therapies thought not to be immunosuppressive (such as extracorporeal photopheresis; ECP), were considered for enrollment in this study on a case-by-case basis. Had persistent active cGVHD manifestations, as defined by 2014 National Institute of Health Consensus Development Project on Criteria for Clinical trials in cGVHD, after at least 2 months of steroid therapy. No more than 3 prior lines of treatment for cGVHD. Karnofsky Performance Scale of greater than (>) 40. Adequate organ and bone marrow functions evaluated during the 14 days prior to enrollment as follows: Absolute neutrophil count greater than or equal to (>=) 1.5×10⁹/L (without myeloid growth factors within 1 week of study entry); Platelet count>=50×10⁹/L (without transfusion or thrombopoietin or thrombopoietin analogues within 2 weeks of study entry); Adequate safety laboratory values: Total bilirubin less than or equal to (<=) 1.5× upper limit of normal (ULN); ALT and AST <=3*ULN; Glomerular filtration rate>=30 milliliter per minute per 1.73 square meter (mL/min/1.73 m²) using the 4-Variable Modification of Diet in Renal Disease variable formula.

Exclusion Criteria. Female participant who was pregnant or breastfeeding. Received an investigational GVHD treatment within 28 days of study entry. Had acute GVHD. Taken any medication known to be a moderate or strong inhibitor of the cytochrome (CY) P3A4 isozyme or any drugs that are moderate or strong CYP3A4 inducers. History or other evidence of severe illness or any other conditions that would make the participant, in the opinion of the investigator, unsuitable for the study (such as poorly controlled psychiatric disease or coronary artery disease). Regular and excessive use of alcohol within the 6 months prior to study entry defined as alcohol intake>14 drinks per week in a man or >7 drinks per week in a woman. Approximately 10 grams of alcohol equals one “drink” unit. One unit equals 1 ounce of distilled spirits, one 12-ounce beer, or one 4-ounce glass of wine. Known history of human immunodeficiency virus or active hepatitis C virus or hepatitis B virus. Diagnosed with another malignancy (other than malignancy for which transplant was performed) within 3 years of enrollment, with the exception of completely resected basal cell or squamous cell carcinoma of the skin, resected in situ cervical malignancy, resected breast ductal carcinoma in situ, or low-risk prostate cancer after curative resection. Relapse of the underlying cancer or post-transplant lymphoproliferative disease at the time of screening. Had previous exposure to belumosudil or known allergy/sensitivity to belumosudil or any other Rho-associated protein kinase 2 inhibitor. Taken other immunosuppressant drugs for GVHD, including mammalian target of rapamycin inhibitors (Note: Only steroids, calcineurin inhibitors, and ECP are acceptable). Corrected QT interval using Fridericia's formula >450 milliseconds.

### Study Design and Treatment

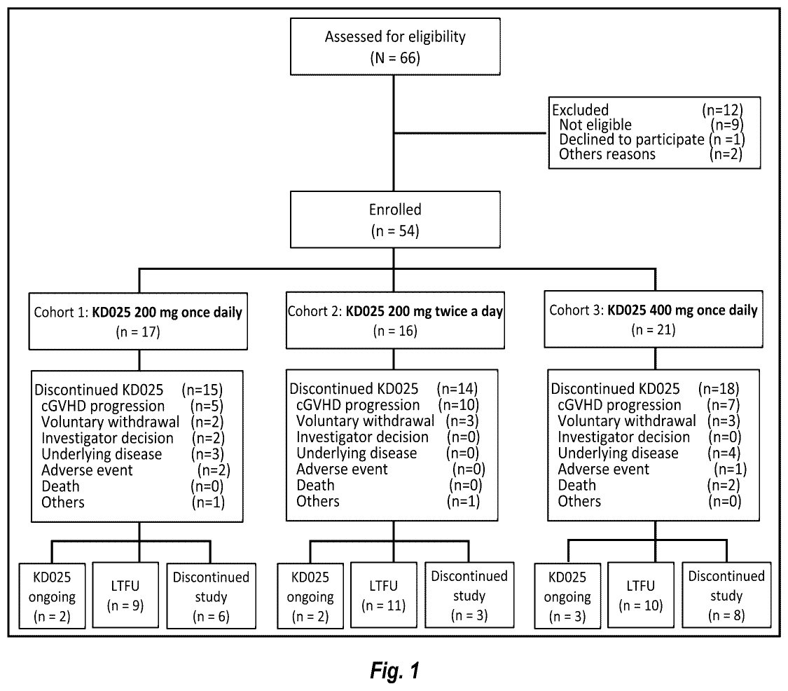

Patients were enrolled into three sequential cohorts: cohort one received belumosudil 200 mg once daily, cohort two received belumosudil 200 mg twice daily (twice a day), and cohort three received belumosudil 400 mg once daily. (FIG. 1) Before enrollment of the subsequent cohort, safety data in each previous cohort were analyzed after eight patients reached two months of treatment to assure that there was no safety signal. The 2-month timeframe was selected because all clinically significant belumosudil-related adverse events (AEs) to date had occurred in ≤36 days of starting belumosudil. No safety concerns were identified, allowing for planned dose escalation.

Belumosudil was administered orally in 28-day cycles until disease progression or unacceptable toxicity. Progression was defined per the 2014 NIH cGVHD Consensus Criteria. Long-term follow-up was conducted every 8 weeks until study closeout. After 4 weeks of belumosudil therapy, corticosteroid therapy could be tapered at the investigators' discretion. Screening was conducted within 28 days of the first study dose. Response was initially assessed after two cycles; however, this was amended to evaluate response on day 1 of each cycle, starting at cycle 2 day 1.

### Study Endpoints

The primary efficacy end point was ORR, defined as the proportion of patients who achieved either a complete response (CR) or partial response (PR), per the 2014 NIH cGVHD Consensus Criteria, at any time point. Only response assessments before the next lines of therapy after belumosudil were counted toward ORR. All responses were assessed by the investigators. Secondary end points included the number and the percentage of patients with steroid-dependent cGVHD who had a best response of PR or CR, duration of response (DOR), response rate by organ system, LSS score, corticosteroid dose reductions, time to next treatment (TTNT), failure-free survival (FFS), and overall survival (OS). The safety and tolerability of belumosudil were evaluated via AE assessments, physical examinations, vital sign measurements, laboratory tests, and electrocardiograms throughout the study. Predose samples were collected for pharmacodynamic (PD) evaluation, which included the assessment of immune cell subtypes in peripheral blood.

### Statistical Analysis

With a sample size of 16 patients per cohort, the study had a >90% probability of ≥1 study participants experiencing an AE with an underlying rate of ≥14%, which was derived from the probability calculations of the assumed sample size. Assuming a best ORR of 25%, which was determined to be clinically meaningful, the study was expected to have approximately 90% probability to show a response in ≥2 patients per cohort. This study was not powered to show significant differences between cohorts with respect to efficacy, AEs, or PD analyses. The primary analysis was conducted using the safety population, defined as enrolled patients who received ≥1 dose of study medication. The Clopper-Pearson (exact) method was used to construct the two-sided 95% CI for ORR. The Kaplan-Meier (K-M) method was used to calculate estimates of FFS and OS.

Results. Subjects

A total of fifty-four patients were enrolled in sequential cohorts: 17 patients in cohort 1, 16 patients in cohort 2, and 21 patients in cohort 3 (FIG. 1). As of the data cutoff for this analysis, the median duration of follow-up was 36 months in cohort 1, 32 months in cohort 2, and 24 months in cohort 3. The overall median duration of follow-up was 29 months (range, 1-39 months).

Demographics and baseline characteristics were overall comparable across cohorts (Table 1, Table 2). The median age at baseline was 52 years (range, 20-75 years). The median time from cGVHD diagnosis to treatment was longest in cohort 1 at 26 months (compared with 18 months and 16 months in cohorts 2 and 3, respectively). Seventy-eight percent of patients had severe cGVHD per investigator assessment. Half of the patients had involvement of ≥4 organs, and more patients in cohort 3 had lung involvement (48%) compared with those in cohorts 1 (24%) and 2 (19%). The baseline median corticosteroid dose (mg/kg/d prednisone equivalent) was 0.22, 0.19, and 0.17 across cohorts, respectively. Patients in cohort 1 had received a median of three prior lines of treatment, whereas patients in cohorts 2 and 3 had received a median of two prior lines of treatment. Seventy-three percent (35 of 48, data not available for six patients) of patients were refractory to their last line of treatment before study enrollment. The CONSORT diagram (FIG. 1) shows patient disposition. The median duration of treatment was 8.5 months (range, 2-39 months) in cohort 1, 7.5 months (range, 1-35 months) in cohort 2, and 9 months (range, 1-29 months) in cohort 3. Twenty-eight percent of patients have received >18 months of belumosudil. Reasons for discontinuing belumosudil included cGVHD progression (n=22), voluntary withdrawal by patients (n=8), relapse of underlying disease (n=7), investigator decision (n=3), AEs considered to be possibly treatment related (n=3), and death (n=2). LTFU means long-term follow up.

TABLE 1
Baseline Demographics and Clinical Characteristics

| | Cohort 1 | | Cohort 2 | | Cohort 3 | | | |
|---|---|---|---|---|---|---|---|---|
| | KD025 200 | | KD025 200 | | KD025 400 | | | |
| | mg Once | | mg Twice a | | mg Once | | | |
| | Daily | | Day | | Daily | | Total | |
| Characteristic | (n = 17) | | (n = 16) | | (n = 21) | | (N = 54) | |
| Median age, years (range) | 50 | (20-63) | 55 | (30-75) | 46 | (25-75) | 52 | (20-75) |
| Male, n (%) | 13 | (77) | 9 | (56) | 12 | (57) | 34 | (63) |
| Indication for transplant, n (%) | | | | | | | | |
| AML | 3 | (18) | 8 | (50) | 9 | (43) | 20 | (37) |
| ALL | 3 | (18) | 2 | (13) | 3 | (14) | 8 | (15) |
| MDS | 2 | (12) | 2 | (13) | 2 | (10) | 6 | (11) |
| Non-Hodgkin lymphoma | 3 | (18) | 0 | | 2 | (10) | 5 | (9) |
| Other non-Hodgkin lymphoma | 0 | | 2 | (13) | 1 | (5) | 3 | (6) |
| Others | 6 | (35) | 2 | (13) | 4 | (19) | 12 | (22) |
| Conditioning intensity, n (%)^{a} | | | | | | | | |
| Myeloablative | 9 | (53) | 5 | (31) | 10 | (48) | 24 | (44) |
| Nonmyeloablative | 7 | (41) | 8 | (50) | 10 | (48) | 25 | (46) |
| Unknown | 1 | (6) | 3 | (19) | 1 | (5) | 5 | (9) |
| Stem-cell source, n (%)^{a} | | | | | | | | |
| Peripheral blood | 15 | (88) | 15 | (94) | 18 | (86) | 48 | (89) |
| Bone marrow | 0 | | 0 | | 1 | (5) | 1 | (2) |
| Cord blood | 1 | (6) | 0 | | 0 | | 1 | (2) |
| Unknown | 1 | (6) | 1 | (6) | 2 | (10) | 4 | (7) |
| HLA matching of donor or | | | | | | | | |
| recipient, n (%)^{a} | | | | | | | | |
| Matched | 14 | (82) | 13 | (81) | 18 | (86) | 45 | (83) |
| Partially matched | 3 | (18) | 3 | (19) | 2 | (10) | 8 | (15) |
| Unknown | 0 | | 0 | | 1 | (5) | 1 | (2) |
| CMV-positive serostatus | | | | | | | | |
| (donor/recipient), n (%) | | | | | | | | |
| +/+ | 4 | (24) | 4 | (25) | 6 | (29) | 14 | (26) |
| +/− | 1 | (6) | 3 | (19) | 0 | | 4 | (7) |
| −/+ | 6 | (35) | 4 | (25) | 6 | (29) | 16 | (30) |
| −/− | 3 | (18) | 4 | (25) | 6 | (29) | 13 | (24) |
| At least 1 unknown | 3 | (18) | 1 | (6) | 3 | (14) | 7 | (13) |
| Median time from cGVHD | 26.4 | (0.0-130.7) | 18.0 | (1.0-69.9) | 16.0 | (1.0-161.9) | 20.0 | (0.0-161.9) |
| diagnosis to enrollment, | | | | | | | | |
| months (range) | | | | | | | | |
| cGVHD severity, n (%)^{b} | | | | | | | | |
| Severe | 12 | (71) | 14 | (88) | 16 | (76) | 42 | (78) |
| Moderate | 5 | (29) | 2 | (13) | 4 | (19) | 11 | (20) |
| Mild | 0 | | 0 | | 1 | (5) | 1 | (2) |
| Organ involvement | | | | | | | | |
| Median No. of organs | 3 | (2-6) | 4 | (1-7) | 3 | (2-7) | 3 | (2-7) |
| involved, n (range) | | | | | | | | |
| ≥4 organs involved, n (%) | 8 | (47) | 10 | (63) | 9 | (43) | 27 | (50) |
| Eyes, n (%) | 14 | (82) | 11 | (69) | 17 | (81) | 42 | (78) |
| Skin, n (%) | 13 | (77) | 12 | (75) | 15 | (71) | 40 | (74) |
| Mouth, n (%) | 13 | (77) | 11 | (69) | 11 | (69) | 35 | (65) |
| Joints and/or fascia, n (%) | 11 | (65) | 11 | (69) | 12 | (57) | 34 | (63) |
| Lungs, n (%) | 4 | (24) | 3 | (19) | 10 | (48) | 17 | (32) |
| Upper GI, n (%) | 2 | (12) | 4 | (25) | 2 | (10) | 8 | (15) |
| Esophagus, n (%) | 2 | (12) | 0 | | 4 | (19) | 6 | (11) |
| Lower GI, n (%) | 1 | (6) | 2 | (13) | 1 | (5) | 4 | (7) |
| Liver, n (%) | 0 | | 2 | (13) | 0 | | 2 | (4) |
| Median Karnofsky | | | | | | | | |
| performance status, n (%) | | | | | | | | |
| ≤50 | 0 | | 0 | | 1 | (5) | 1 | (2) |
| 60-70 | 4 | (24) | 4 | (25) | 6 | (29) | 14 | (26) |
| 80-90 | 13 | (77) | 12 | (75) | 14 | (67) | 39 | (72) |
| 100 | 0 | | 0 | | 0 | | 0 | |
| Prior therapy characteristics | | | | | | | | |
| Median prior LOTs, n | 3 | | 2 | | 2 | | 3 | |
| ≥2 prior LOTs, n (%) | 15 | (88) | 9 | (56) | 14 | (67) | 38 | (70) |
| Refractory to prior LOT, | 11/15 | (73) | 9/13 | (69) | 15/20 | (75) | 35/48 | (73) |
| n (%)^{a} | | | | | | | | |

Abbreviations: ALL, acute lymphocytic leukemia; AML, acute myelogenous leukemia; cGVHD, chronic graft-versus-host disease; CMV, cytomegalovirus; LOT, line of therapy; MDS, myelodysplastic syndrome.
ªDenominator excludes patients with unknown status (six patients in total).
^{b}Disease severity was determined using the Physician-reported Global cGVHD Activity Assessment Form.

TABLE 2
Additional Baseline Demographics

| | Cohort 1 | | Cohort 2 | | Cohort 3 | | | |
|---|---|---|---|---|---|---|---|---|
| | KD025 200 | | KD025 200 | | KD025 400 | | | |
| | mg Once | | mg Day | | mg Once | | | |
| | Daily | | Twice a Day | | Daily | | Total | |
| Characteristic | (n = 17) | | (n = 16) | | (n =21) | | (N = 54) | |
| Prior systemic cGVHD therapy | | | | | | | | |
| type, No. (%) | | | | | | | | |
| CS | 17 | (100) | 16 | (100) | 21 | (100) | 54 | (100) |
| Tacrolimus | 8 | (47) | 7 | (44) | 11 | (52) | 26 | (48) |
| Sirolimus | 10 | (59) | 8 | (50) | 6 | (29) | 24 | (44) |
| Rituximab | 8 | (47) | 3 | (19) | 5 | (24) | 16 | (30) |
| ECP | 5 | (29) | 4 | (25) | 6 | (29) | 15 | (28) |
| MMF | 4 | (24) | 4 | (25) | 4 | (19) | 12 | (22) |
| Cyclosporine | 3 | (18) | 0 | | 2 | (10) | 5 | (9) |
| Ibrutinib | 1 | (6) | 0 | | 3 | (14) | 4 | (7) |
| MTX | 1 | (6) | 2 | (13) | 0 | | 3 | (6) |
| Ixazomib | 1 | (6) | 1 | (6) | 0 | | 2 | (4) |
| ATG | 1 | (6) | 0 | | 0 | | 1 | (2) |
| Ofatumumab | 0 | | 0 | | 1 | (5) | 1 | (2) |
| Imatinib | 1 | (6) | 0 | | 0 | | 1 | (2) |
| Ruxolitinib | 0 | | 0 | | 1 | (5) | 1 | (2) |
| Continuing systemic cGVHD | | | | | | | | |
| therapy type | | | | | | | | |
| CS, No. (%) | 17 | (100) | 16 | (100) | 21 | (100) | 54 | (100) |
| Mean prednisone equivalent dose | 0.22 | | 0.24 | | 0.28 | | 0.25 | |
| at enrollment, mg/kg/d | | | | | | | | |
| CNI, No. (%) | 7 | (41) | 6 | (38) | 12 | (57) | 25 | (46) |
| ECP, No. (%) | 4 | (24) | 4 | (25) | 4 | (19) | 12 | (22) |

Abbreviations: ATG, antithymocyte globulin; cGVHD, chronic graft-versus-host disease; CNI, calcineurin inhibitor; CS, corticosteroid; ECP, extracorporeal photopheresis; MMF, mycophenolate mofetil; MTX, methotrexate.

### Efficacy

Overall response rate. In the safety population (N=54), the ORR (95% CI) was 65% (51% to 77%). The ORR (95% CI) was similar across cohorts: 65% (38% to 86%) in cohort 1, 69% (41% to 89%) in cohort 2, and 62% (38% to 82%) in cohort 3 (Table 3). Efficacy data for subgroups and secondary end points are presented as pooled data across cohorts.

TABLE 3
Efficacy and CS Reduction

| | | Cohort 2 | | |
|---|---|---|---|---|
| | Cohort 1 | KD025 200 | Cohort 3 | |
| | KD025 200 mg | mg Twice a | KD025 400 mg | |
| | Once Daily | Day | Once Daily | Total |
| Characteristic | (n = 17) | (n = 16) | (n = 21) | (N = 54) |
| ORR, % (95% CI) | 65 | 69 | 62 | 65 |
| | (38 to 86) | (41 to 89) | (38 to 82) | (51 to 77) |
| Subgroup analyses, | | | | |
| n/N (%, 95% CI) | | | | |
| ≤2 prior LOTs | 10/15 | 5/8 | 8/12 | 23/35 |
| | (67, 38 to 88) | (63, 25 to 92) | (67, 35 to 90) | (66, 48 to 81) |
| Refractory to | 7/11 | 6/9 | 9/15 | 22/35 |
| previous LOT | (64, 31 to 89) | (67, 30 to 93) | (60, 32 to 94) | (63, 45 to 79) |
| ≤4 organs involved | 4/8 | 8/10 | 7/9 | 19/27 |
| | (50, 16 to 84) | (80, 44 to 98) | (78, 40 to 97) | (70, 50 to 86) |
| Severe cGVHD^{a} | 8/12 | 9/14 | 8/16 | 25/42 |
| | (67, 35 to 90) | (64, 35 to 87) | (50, 25 to 75) | (60, 43 to 74) |
| Clinically significant | | | | |
| improvement (LSS)^{b} | | | | |
| Overall, n (%, 95% | 9 | 7 | 11 | 27 |
| CI) | (53, 28 to 77) | (44, 20 to 70) | (52, 30 to 74) | (50, 36 to 64) |
| Responder, n/N (%, | 8/11 | 3/11 | 9/13 | 20/35 |
| 95% CI) | (73, 39 to 94) | (27, 6 to 61) | (69, 39 to 91) | (57, 39 to 74) |
| Nonresponder, n/N | 1/6 | 4/5 | 2/8 | 7/19 |
| (%, 95% CI) | (17, 0.4 to 64) | (80, 28 to 99) | (25, 3 to 65) | (37, 16 to 62) |
| Proportion with CS | 13 | 9 | 14 | 36 |
| reduction, n (%, 95% | (76, 50 to 93) | (56, 30 to 80) | (67, 43 to 85) | (67, 53 to 79) |
| CI) | | | | |
| Mean percent | | | | |
| change in CS dose | | | | |
| from baseline, % | | | | |
| Overall | −50 | −36 | −47 | −45 |
| Responder | −63 | −36 | −63 | −55 |
| Nonresponder | −26 | −37 | −19 | −26 |
| CS discontinuation, n | 4 | 2 | 4 | 10 |
| (%, 95% CI) | (24, 7 to 50) | (13, 2 to 38) | (19, 5 to 42) | (19, 9 to 31) |

Abbreviations: cGVHD, chronic graft-versus-host disease; CS, corticosteroid; LOT, line of therapy; LSS, Lee Symptom Scale; ORR, overall response rate.
^{a}Disease severity was determined using the Physician-reported Global cGVHD Activity Assessment Form.
^{b}Changes in cGVHD symptom burden were measured by the LSS. Clinically meaningful improvement in symptom burden was defined as a decrease of at least seven points in LSS summary score.

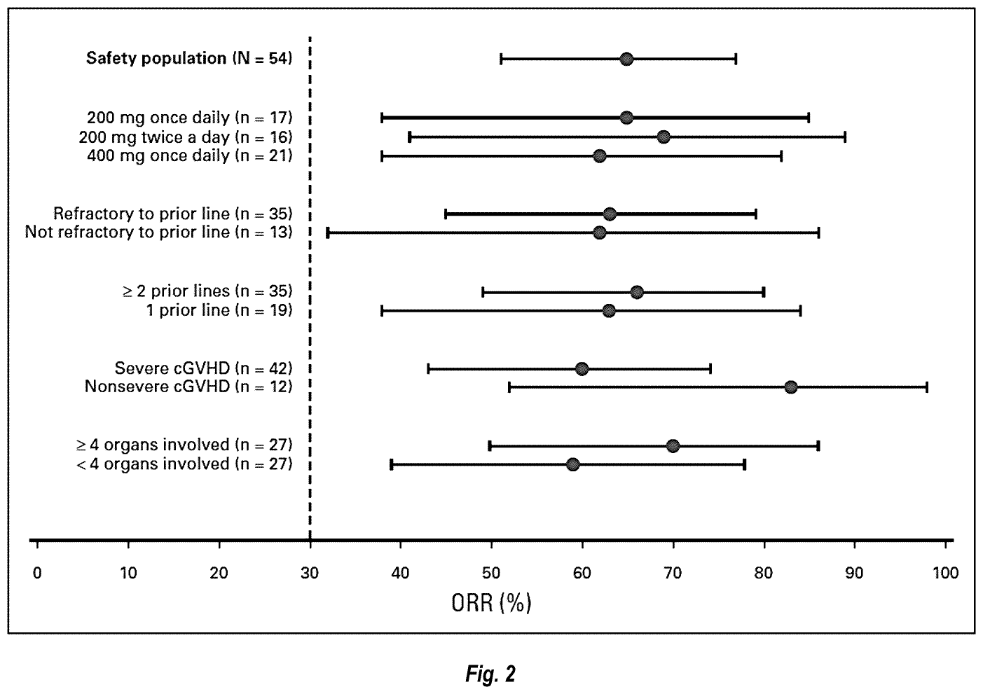

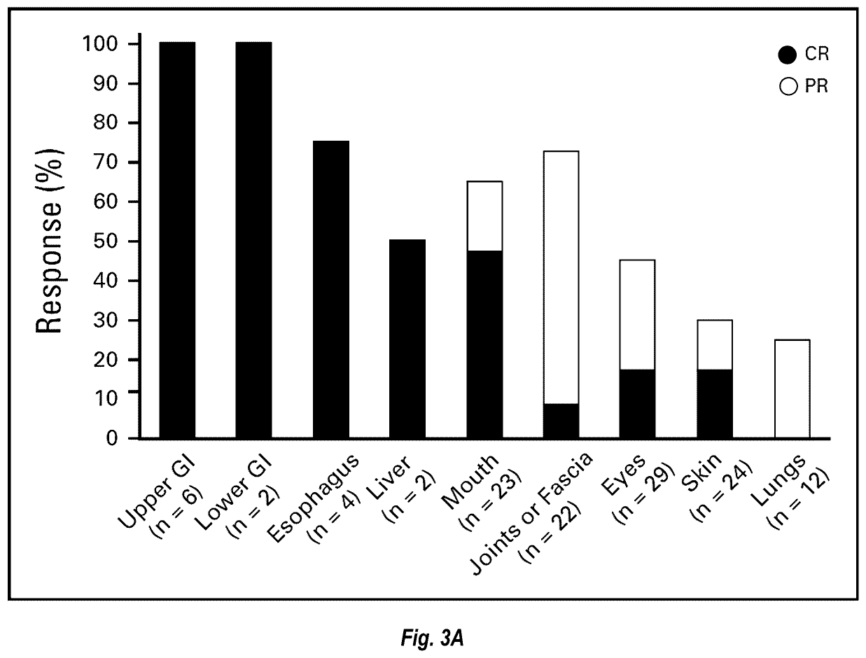

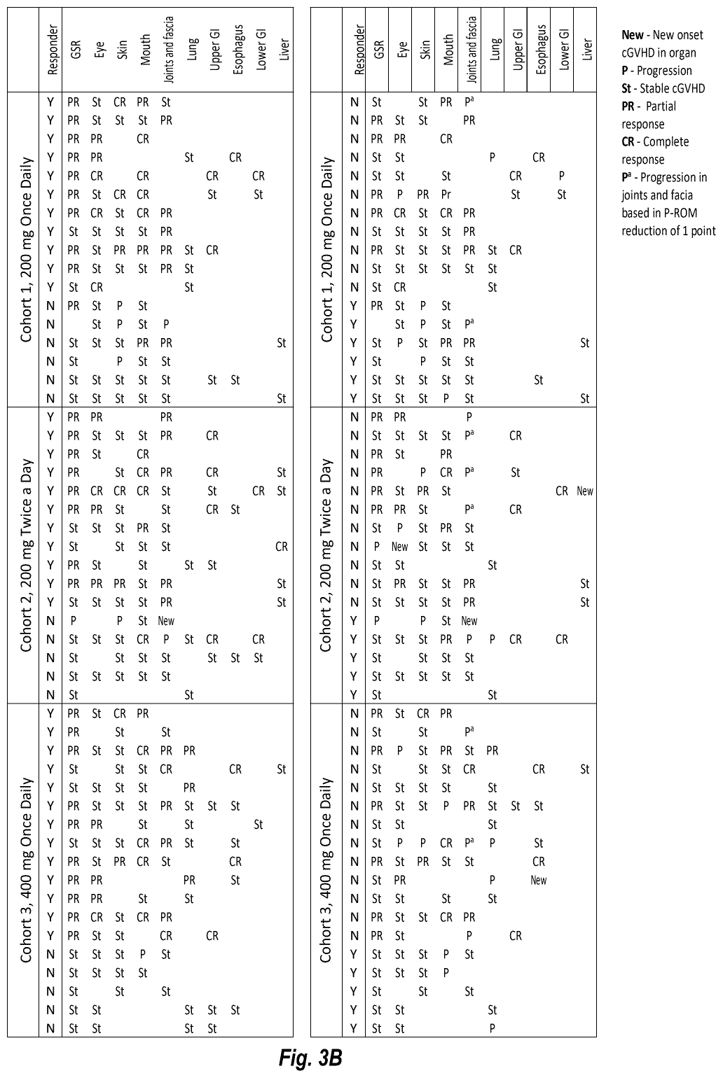

Responses were achieved across key subgroups, with ORRs of 60% (25 of 42) in patients with severe cGVHD, 66% (23 of 35) in patients who had received ≥2 prior systemic lines of therapy, 63% (22 of 35) in patients who were refractory to their last lines of therapy before enrollment, and 70% (19 of 27) in patients with ≥4 organs involved (FIG. 2). All responses at the patient level were PR; however, organ specific analyses showed that CR was achieved across all affected organs, with the exception of the lungs, where PR was the best response achieved (FIG. 3A and FIG. 3B). FIG. 3B shows for the best response by organ that three partial responses were achieved in lung at the 400 mg once daily dose.

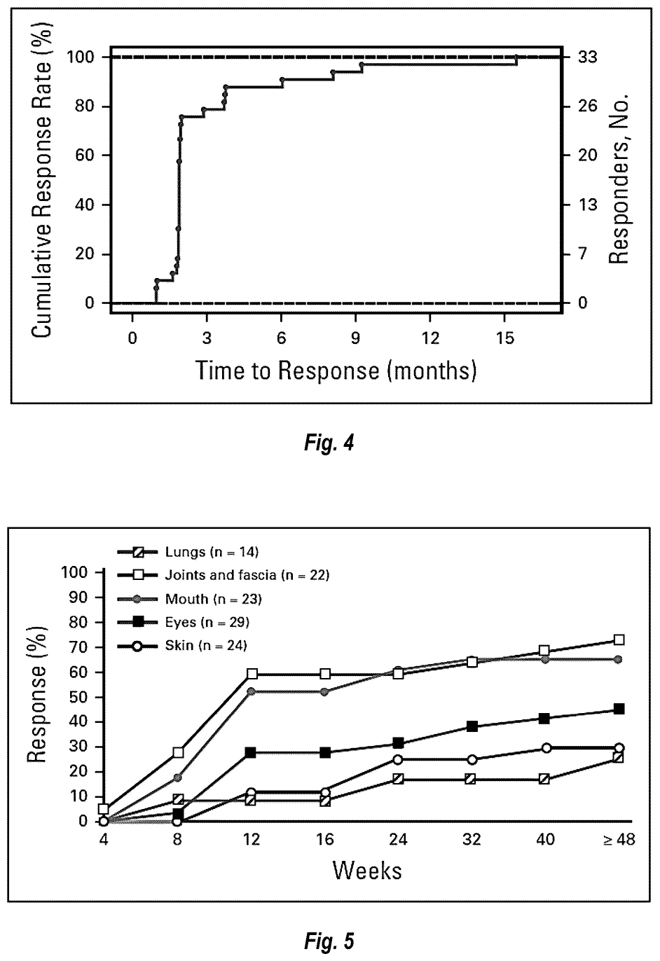

Responses were generally rapid, with >75% of all responses achieved by the first response assessment at week 8 (FIG. 4). Four of 35 responses occurred after 24 weeks of belumosudil treatment, with late organ responses observed in the lungs, joints and/or fascia, and eyes (FIG. 5).

Percentage of Participants With Overall Response (OR): OR was defined as the percentage of participants with complete response (CR) or partial response (PR). The OR determination of chronic graft versus host disease (cGVHD) was based on cGVHD response assessment performed by clinicians as per the 2014 National Institutes of Health (NIH) Consensus Development Project for Clinical Trials in cGVHD criteria. CR was defined as the resolution of all manifestations in each organ or site. PR was defined as the improvement in at least 1 organ or site without progression in any other organ or site; and cGVHD progression was defined as the clinically meaningful worsening in 1 or more organs regardless of improvement in other organs. Timeframe: From the date of first response until documented disease progression or death due to any cause or data cut-off, whichever occurred first (maximum duration: up to 64.2 months.

Analysis was performed on mITT population. Participants received belumosudil orally on Day 1, 8, 15, and 22 of each 28-day treatment cycle until disease progression, unacceptable toxicity, or death whichever occurred first (maximum duration: 64.2 months). The data is shown in the table below.

TABLE 4
OR

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number of | 17 | 16 | 21 |
| Participants Analyzed | | | |
| Percentage of | 65 (38-86%) | 69 (41-89%) | 57 (34-78%) |
| Participants With | | | |
| Overall Response | | | |
| Number (95% | | | |
| Confidence Interval) | | | |
| Unit of Measure = | | | |
| percentage of | | | |
| participants | | | |

Duration of Response (DOR). The DOR was defined as the time (in weeks) from first documentation of response to the time of first documentation of deterioration from best response (e.g., CR to PR, or PR to LR). LOR included the response status of unchanged (LOR-U), mixed (LOR-M), or progression (LOR-P). Per the 2014 NIH Consensus Development Project for Clinical Trials in cGVHD criteria; CR was defined as the resolution of all manifestations in each organ or site; PR was defined as the improvement in at least 1 organ or site without progression in any other organ or site; LOR-M was defined as CR or PR in at least one organ accompanied by progression in another organ, LOR-U was defined as outcomes that did not meet the criteria for CR, PR, progression or mixed response, LOR-P was defined as progression in at least one organ or site without a response in any other organ or site. Kaplan-Meier was used for the analysis. Timeframe: From the date of first response until documented disease progression or death due to any cause or data cut-off, whichever occurred first (maximum duration: up to 64.2 months.

Analysis was performed on responder population which included participants who received at least 1 dose of study medication and achieved a PR or CR response at any post-baseline response assessment. Participants received belumosudil orally on Day 1, 8, 15, and 22 of each 28-day treatment cycle until disease progression, unacceptable toxicity, or death whichever occurred first (maximum duration: 64.2 months). The data is shown in the table below.

TABLE 5
DOR

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number of | 11 | 11 | 12 |
| Participants Analyzed | | | |
| DOR | 40 (8-na* %) | 11 (2-35%) | 16 (4-38%) |
| Median (95% | | | |
| Confidence Interval) | | | |
| Unit of Measure = | | | |
| weeks | | | |

*95% CI upper limit not available due to 3 (27.3%) participants were censored

Time to next therapy (TTNT). The TTNT was defined as the time (in months) from first treatment to the time of new systemic cGVHD treatment. TTNT was censored by last response assessment or long term follow up assessment, whichever was earlier. Kaplan-Meier survival method was used for the analysis. Timeframe: From time of first treatment to the time of new systemic cGVHD treatment or long term follow-up assessment, whichever occurred first (maximum duration: up to 64.2 months). Analysis was performed on mITT population. The data is shown in the table below.

TABLE 6
TTNT

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number of | 17 | 16 | 21 |
| Participants Analyzed | | | |
| TTNT | 15 (5-NA* %) | 10 (4-19%) | 14 (3-NA %) |
| Median (95% | | | |
| Confidence Interval) | | | |
| Unit of Measure = | | | |
| months | | | |

*95% confidence interval upper limit are not available due to high percentage of censoring.

Subsequent systemic cGVHD therapies included tacrolimus, sirolimus, ibrutinib, ruxolitinib, extracorporeal photopheresis, and mycophenolate mofetil.

FFS and OS. Failure-free Survival (FFS) and Overall Survival (OS)

Failure-free survival was defined as the time (in months) from first dose of study drug to either the start of another new systemic treatment for cGVHD, relapse of the underlying disease or death. If no such events happened, FFS was censored by last response assessment or long term follow up assessment, whichever was the latest and available. Kaplan-Meier survival method was used for the analysis. Analysis was performed on mITT population. Timeframe: From first dose of study drug to either start of another new systemic treatment for cGVHD, relapse of the underlying disease or death or data cut-off, whichever occurred first (maximum duration: up to 64.2 months). Participants received belumosudil orally on Day 1, 8, 15, and 22 of each 28-day treatment cycle until disease progression, unacceptable toxicity, or death whichever occurred first (maximum duration: 64.2 months). The data is shown in the table below.

TABLE 7
FFS

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number of | 17 | 16 | 21 |
| Participants Analyzed | | | |
| FFS | 11 (4-na* %) | 10 (4-19%) | 10 (3-16%) |
| Median (95% | | | |
| Confidence Interval) | | | |
| Unit of Measure = | | | |
| months | | | |

*not specified

Overall survival was defined as the time (in months) from first dose of study drug to the death due to any reason. If there was no death, OS was censored by last visit, last long term follow-up, or study cutoff date, whichever was the latest. Kaplan-Meier survival method was used for the analysis. Timeframe: From first dose of study drug to date of death from any cause or data cut-off, whichever occurred first (maximum duration: up to 64.2 months). Participants received belumosudil orally on Day 1, 8, 15, and 22 of each 28-day treatment cycle until disease progression, unacceptable toxicity, or death whichever occurred first (maximum duration: 64.2 months). The data is shown in the table below.

TABLE 8
OS

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number of | 17 | 16 | 21 |
| Participants Analyzed | | | |
| OS | NA | NA | NA |
| Median (95% | (40-NA* %) | (52-NA %) | (23-NA %) |
| Confidence Interval) | | | |
| Unit of Measure = | | | |
| months | | | |

*OS medians and the corresponding 95% upper limits are not available due to the majority of the participants were still alive at the time of the final analysis.

QOL assessment. Clinically meaningful improvement in LSS score, defined as a decrease of ≥7 points in the LSS summary score, during belumosudil treatment was observed in 50% of patients. Thirty-five percent of all patients (37% of responders and 32% of nonresponders) reported a clinically meaningful improvement in LSS score on consecutive assessments.

Corticosteroid sparing. During belumosudil treatment, 67% of patients reduced corticosteroid dose and 19% completely discontinued corticosteroid therapy. The mean corticosteroid dose was reduced by 45%. The median time to corticosteroid therapy discontinuation was 29 weeks (range, 8-77 weeks). The mean corticosteroid dose reduction was 55% in responders and 26% in nonresponders (Table 3).

Best Overall Response (BOR). BOR was defined as the participants with either a CR or PR or lack of response (LOR), where LOR included the response status of unchanged (LOR-U), mixed (LOR-M), or progression (LOR-P). BOR was assessed per the 2014 NIH Consensus Development Project for Clinical Trials in cGVHD criteria. CR was defined as the resolution of all manifestations in each organ or site; PR was defined as the improvement in at least 1 organ or site without progression in any other organ or site; LOR-M was defined as CR or PR in at least one organ accompanied by progression in another organ, LOR-U was defined as outcomes that did not meet the criteria for CR, PR, progression or mixed response, LOR-P was defined as progression in at least one organ or site without a response in any other organ or site. Timeframe: From the date of randomization to the date of first documentation of progression or death due to any cause or data cut-off, whichever occurred first (maximum duration: up to 64.2 months). Analysis was performed on mITT population. The data is shown in the table below.

TABLE 9
BOR

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number of | 17 | 16 | 21 |
| Participants Analyzed | | | |
| Measure Type: Count | | | |
| of Participants | | | |
| Unit of measure = | | | |
| participants | | | |
| CR | 0 (0%) | 0 (0%) | 0 (0%) |
| PR | 11 (65%) | 11 (69%) | 12 (57%) |
| LOR-U | 2 (12%) | 3 (19%) | 4 (19%) |
| LOR-M | 2 (12%) | 1 (6%) | 0 (0%) |
| LOR-P | 2 (12%) | 1 (6%) | 1 (5%) |

Maximal Improvement from Baseline in Global Severity Rating (GSR) by Clinician-reported cGVHD Assessment. The GSR assessment was performed by asking the participants to rate their disease severity of cGVHD symptoms on a 0 to 10-point numeric rating scale, where score 0 indicated ‘not at all severe cGVHD symptoms’ and score 10 indicated ‘most severe cGVHD symptoms possible’. The response was defined using the scores from 9 organs: skin, eyes, mouth, esophagus, upper gastrointestinal (GI) track, lower GI tract, liver, lungs, and joints and fascia plus GSR. End of treatment (EOT) visit was performed within 3 days after the participant's last dose of study drug. Baseline value was defined as valid and last non-missing value obtained within 28 days prior to participant receiving first study medication. Timeframe: From Baseline up to end of treatment (i.e., up to 64.2 months). Analysis was performed on mITT population. Participants received belumosudil 200 mg orally QD on Day 1, 8, 15, and 22 of each 28-day treatment cycle until disease progression, unacceptable toxicity, or death whichever occurred first (maximum duration: 64.2 months). The data is shown in the table below.

TABLE 10
GSR

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number of | 17 | 16 | 21 |
| Participants Analyzed | | | |
| Measure Type: Count | | | |
| of Participants | | | |
| Unit of measure = | | | |
| participants | | | |
| −7 | 0 (0%) | 0 (0%) | 2 (10%) |
| −6 | 0 (0%) | 2 (13%) | 0 (0%) |
| −5 | 2 (12%) | 2 (13%) | 0 (0%) |
| −4 | 2 (12%) | 0 (0%) | 3 (14%) |
| −3 | 4 (24%) | 0 (0%) | 1 (5%) |
| −2 | 2 (12%) | 4 (25%) | 4 (19%) |
| −1 | 1 (6%) | 2 (13%) | 4 (19%) |
| 0 | 5 (29%) | 4 (25%) | 3 (14%) |
| 1 | 0 (0%) | 1 (6%) | 1 (5%) |
| 3 | 0 (0%) | 1 (6%) | 0 (0%) |
| missing | 1 (6%) | 0 (0%) | 3 (14%) |

Number of Participants With Maximal Improvement From Baseline in Symptom Activity by cGVHD Activity Assessment Participant Self-Report. cGVHD symptom severity was self-reported by participants. Participants were asked to rate their disease symptom severity over the last week on the following questions: skin itching at its worst, moth dryness at its worst, mouth pain at its worst, mouth sensitivity at its worst, main compliant on eyes, symptom severity on eyes. The severity rating was done on a 0 to 10-point numeric rating scare, where score 0 indicated ‘not at all severe cGVHD symptoms’ and score 10 indicated ‘most severe cGVHD symptoms possible’. EOT visit was performed within 3 days after the participant's last dose of study drug. Baseline value was defined as valid and last non-missing value obtained within 28 days prior to participant receiving first study medication. Timeframe: From Baseline up to end of treatment (i.e., up to 64.2 months). Analysis was performed on mITT population. The data is shown in the table below.

TABLE 11
Number of Participants With Maximal Improvement
From Baseline in Symptom Activity by cGVHD
Activity Assessment Participant Self-Report

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Number of Participants | 17 | 16 | 21 |
| With Maximal | | | |
| Improvement From | | | |
| Baseline in Symptom | | | |
| Activity by cGVHD | | | |
| Activity Assessment | | | |
| Participant Self-Report | | | |
| Measure Type: Count | | | |
| of Participants | | | |
| Unit of measure = | | | |
| participants | | | |
| −8 | 0 (0%) | 0 (0%) | 1 (5%) |
| −7 | 3 (18%) | 2 (13%) | 0 (0%) |
| −6 | 0 (0%) | 1 (6%) | 4 (19%) |
| −5 | 2 (12%) | 0 (0%) | 2 (10%) |
| −4 | 2 (12%) | 2 (13%) | 1 (5%) |
| −3 | 2 (12%) | 2 (13%) | 2 (10%) |
| −2 | 2 (12%) | 3 (19%) | 2 (10%) |
| −1 | 2 (12%) | 2 (13%) | 2 (10%) |
| 0 | 1 (6%) | 1 (6%) | 0 (0%) |
| 1 | 0 (0%) | 0 (0%) | 1 (5%) |
| 2 | 0 (0%) | 1 (6%) | 2 (10%) |
| 4 | 1 (6%) | 0 (0%) | 0 (0%) |
| missing | 2 (12%) | 2 (13%) | 4 (19%) |

Best Response in Each Individual Organ. Best response was defined as the percentage of participants with CR or PR. Response was assessed per the 2014 NIH Consensus Development Project for Clinical Trials in cGVHD criteria; CR was defined as the resolution of all manifestations in each organ or site; PR was defined as the improvement in at least 1 organ or site without progression in any other organ or site. Organ response assessment was performed on 9 individual organs: skin, eyes, mouth, esophagus, upper GI, lower GI, liver, lungs, and joints and fascia. Timeframe: From date of randomization until disease progression or data cut-off, whichever occurred first (maximum duration: up to 64.2 months). Analysis was performed on mITT population. Here, ‘number analyzed’=participants with available data for each specified category. Here, ‘0’ in the number analyzed field signifies that none of the participants were available for the analysis at the specified time points. The data is shown in the table below:

TABLE 12
Best Response in Each Individual Organ

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number of | 17 | 16 | 21 |
| Participants Analyzed | | | |
| participant number | | | |
| analyzed | | | |
| (percentage of | | | |
| participants with best | | | |
| response in each | | | |
| individual organ) | | | |
| Skin | 13 participants | 12 participants | 15 participants |
| | (23%) | (17%) | (13%) |
| Eyes | 14 participants | 11 participants | 17 participants |
| | (36%) | (36%) | (24%) |
| Mouth | 13 participants | 11 participants | 11 participants |
| | (46%) | (46%) | (46%) |
| Esophagus | 2 participants | 0 participants | 4 participants |
| | (50%) | | (25%) |
| Upper GI tract | 2 participants | 4 participants | 2 participants |
| | (100%) | (100%) | (50%) |
| Lower GI tract | 1 participant | 2 participants | 1 participant |
| | (0%) | (100%) | (0%) |
| Liver | 0 participants | 2 participants | 0 participants |
| | | (50%) | |
| Lungs | 5 participants | 3 participants | 10 participants |
| | (0%) | (0%) | (30%) |
| Joints and Fascia | 11 participants | 11 participants | 12 participants |
| | (55%) | (46%) | (42%) |

### Safety

Belumosudil was well-tolerated, with >56 patient-years of belumosudil exposure. The median relative dose intensity was 98% overall. The percentage of patients with a relative dose intensity>95% was 77%, 63%, and 71% across cohorts, respectively. Dose reductions occurred in 9% of patients, and the median duration of reduction was 97 days (range, 21-859 days). Dose interruptions occurred in 41% of patients, and the median duration of interruption was 10 days (range, 2-39 days).

AEs were consistent with those expected in a population of patients with advanced cGVHD receiving corticosteroid therapy. AEs reported in ≥20% of patients were upper respiratory infection (46%), diarrhea (33%), fatigue (33%), nausea (33%), increased liver function tests (33%), dyspnea (30%), headache (24%), peripheral edema (24%), cough (22%), and hypertension (20%) (Table 13). Serious AEs were reported in 43% of patients, and serious AEs reported in >1 patient were dyspnea (7%), lung infection (6%), hypoxia (4%), and influenza-like illness (4%). Sixty-one percent of patients had a grade≥3 AE, with the most common being dyspnea (13%), increased liver function tests (7%), hyperglycemia (7%), and hypoxia (7%) (Table 13). Grade≥3 cytopenias were reported in two patients (4%). These occurred at relapse of underlying malignancy in patients who had otherwise maintained normal blood counts during their belumosudil treatment.

TABLE 13
Safety Overview

| | Cohort 1 | Cohort 2 | Cohort 3 | |
|---|---|---|---|---|
| | KD025 | KD025 | KD025 | |
| | 200 mg | 200 mg | 400 mg | |
| | Once Daily | Twice a Day | Once Daily | Total |
| AE, No. (%) | (n = 17) | (n = 16) | (n = 21) | (N = 54) |
| Any AE | 17 (100) | 16 (100) | 16 (100) | 53 (98) |
| Grade ≥3 AE | 9 (53) | 10 (63) | 14 (67) | 33 (61) |
| Drug-related AE | 8 (47) | 8 (50) | 14 (67) | 30 (56) |
| SAE | 5 (29) | 6 (38) | 12 (57) | 23 (43) |
| Death | 0 | 0 | 2 (10) | 2 (4) |
| Drug-related SAE | 0 | 0 | 0 | 0 |
| All grade in ≥20% | | | | |
| URI | 9 (53) | 9 (56) | 7 (33) | 25 (46) |
| Diarrhea | 6 (35) | 5 (31) | 7 (33) | 18 (33) |
| Nausea | 6 (35) | 4 (25) | 8 (38) | 18 (33) |
| Fatigue | 6 (35) | 3 (19) | 9 (43) | 18 (33) |
| ALT/AST | 11 (65) | 5 (31) | 2 (10) | 18 (33) |
| increased | | | | |
| Dyspnea | 3 (18) | 6 (38) | 7 (33) | 16 (30) |
| Peripheral edema | 3 (18) | 4 (25) | 6 (29) | 13 (24) |
| Headache | 4 (24) | 3 (19) | 6 (29) | 13 (24) |
| Cough | 1 (6) | 4 (25) | 7 (33) | 12 (22) |
| Hypertension | 5 (29) | 2 (13) | 4 (19) | 11 (20) |
| Grade ≥3 in ≥5% | | | | |
| Dyspnea | 1 (6) | 2 (13) | 4 (19) | 7 (13) |
| Lung infection or | 1 (6) | 2 (13) | 2 (10) | 5 (9) |
| pneumonia | | | | |
| ALT/AST | 2 (12) | 2 (13) | 0 | 4 (7) |
| increased | | | | |
| Hyperglycemia | 2 (12) | 0 | 2 (10) | 4 (7) |
| Hypoxia | 1 (6) | 1 (6) | 2 (10) | 4 (7) |
| Anemia | 2 (12) | 1 (6) | 0 | 3 (6) |

Abbreviations: AE, adverse event; SAE, serious adverse event; URI, upper respiratory tract infection.

No cases of cytomegalovirus (CMV) infection or reactivation were reported with belumosudil. Three patients discontinued belumosudil because of potentially drug-related AEs (cohort 1: diarrhea and headache; cohort 3: fatigue). Four patients, all in cohort 3, died during the study (secondary to relapse of leukemia, pneumonia (unknown pathogen), cardiac arrest, and cGVHD progression) with none of the deaths attributed to belumosudil. There was no dose response with respect to the observed AEs.

Reported Adverse Events. Timeframe: From first dose of study drug up to 28 days after the last dose of study drug (maximum duration: up to 64.2 months). Reported AEs and deaths were treatment emergent AEs that developed, worsened, or became serious from first dose of study drug up to 28 days after the last dose of study drug. All-cause mortality data collected during the study were assessed for all enrolled participants. Disease progression related death was not reported as AE. Participants received belumosudil 200 mg orally QD on Day 1, 8, 15, and 22 of each 28-day treatment cycle until disease progression, unacceptable toxicity, or death whichever occurred first (maximum duration: 64.2 months). The data is shown in the three tables below.

TABLE 14
All-Cause Mortality

| | Cohort 1 200 | | Cohort 2 | | Cohort 3 | |
|---|---|---|---|---|---|---|
| | mg QD | | 200 mg BID | | 400 mg QD | |
| | Affected/ | | Affected/ | | Affected/ | |
| | At | # | At | # | At | # |
| | Risk (%) | Events | Risk (%) | Events | Risk (%) | Events |
| Total | 2/17 | | 3/16 | | 3/21 | |
| All- | (11.76%) | | (18.75%) | | (14.29%) | |
| Cause | | | | | | |
| Mortality | | | | | | |

TABLE 15
Serious Adverse Events

| | Cohort 1 200 mg QD | | Cohort 2 200 mg BID | | Cohort 3 400 mg QD | |
|---|---|---|---|---|---|---|
| | Affected/At | # | Affected/At | # | Affected/At | # |
| | Risk (%) | Events | Risk (%) | Events | Risk (%) | Events |
| Total | 5/17 (29.41%) | | 6/16 (37.5%) | | 13/21 (61.9%) | |

Blood and lymphatic system disorders

| | | | | | | |
|---|---|---|---|---|---|---|
| Febrile neutropenia ^{A} † | 0/17 (0%) | 0 | 0/16 (0%) | 0 | 1/21 (4.76%) | 1 |

Cardiac disorders

| | | | | | | |
|---|---|---|---|---|---|---|
| Cardiac arrest ^{A} † | 0/17 (0%) | 0 | 0/16 (0%) | 0 | 1/21 (4.76%) | 1 |
| Pericardial effusion ^{A} † | 0/17 (0%) | 0 | 0/16 (0%) | 0 | 1/21 (4.76%) | 1 |

Gastrointestinal disorders

| | | | | | | |
|---|---|---|---|---|---|---|
| Abdominal wall haematoma ^{A} † | 0/17 (0%) | 0 | 0/16 (0%) | 0 | 1/21 (4.76%) | 1 |
| Small intestinal obstruction ^{A} † | 0/17 (0%) | 0 | 0/16 (0%) | 0 | 1/21 (4.76%) | 1 |

General disorders

| | | | | | | |
|---|---|---|---|---|---|---|
| Influenza like illness ^{A} † | 0/17 (0%) | 0 | 0/16 (0%) | 0 | 2/21 (9.52%) | 2 |
| Non-cardiac chest pain ^{A} † | 0/17 (0%) | 0 | 1/16 (6.25%) | 2 | 0/21 (0%) | 0 |

Immune system disorders

| | | | | | | |
|---|---|---|---|---|---|---|
| Anaphylactic reaction ^{A} † | 1/17 (5.88%) | 1 | 0/16 (0%) | 0 | 0/21 (0%) | 0 |
| Chronic graft versus host disease in skin ^{A} † | 0/17 (0%) | 0 | 1/16 (6.25%) | 1 | 0/21 (0%) | 0 |
| Graft versus host disease in lung ^{A} † | 0/17 (0%) | 0 | 0/16 (0%) | 0 | 1/21 (4.76%) | 1 |

Infections and infestations

| | | | | | | |
|---|---|---|---|---|---|---|
| Bacteraemia ^{A} † | 0/17 (0%) | 0 | 1/16 (6.25%) | 1 | 0/21 (0%) | 0 |
| Cellulitis ^{A} † | 0/17 (0%) | 0 | 1/16 (6.25%) | 1 | 0/21 (0%) | 0 |
| Citrobacter infection ^{A} † | 0/17 (0%) | 0 | 1/16 (6.25%) | 1 | 0/21 (0%) | 0 |
| Pneumonia ^{A} † | 1/17 (5.88%) | 2 | 1/16 (6.25%) | 1 | 3/21 (14.29%) | 3 |
| Pneumonia viral ^{A} † | 0/17 (0%) | 0 | 0/16 (0%) | 0 | 1/21 (4.76%) | 1 |
| Stenotrophomonas infection ^{A} † | 0/17 (0%) | 0 | 1/16 (6.25%) | 1 | 0/21 (0%) | 0 |
| Urinary tract infection ^{A} † | 0/17 (0%) | 0 | 1/16 (6.25%) | 1 | 0/21 (0%) | 0 |
| Varicella zoster virus infection ^{A} † | 0/17 (0%) | 0 | 0/16 (0%) | 0 | 1/21 (4.76%) | 1 |

Investigations

| | | | | | | |
|---|---|---|---|---|---|---|
| Influenza A virus test positive ^{A} † | 1/17 (5.88%) | 1 | 0/16 (0%) | 0 | 0/21 (0%) | 0 |
| Respirovirus test positive ^{A} † | 0/17 (0%) | 0 | 1/16 (6.25%) | 1 | 0/21 (0%) | 0 |

Metabolism and nutrition disorders

| | | | | | | |
|---|---|---|---|---|---|---|
| Lipomatosis ^{A} † | 1/17 (5.88%) | 1 | 0/16 (0%) | 0 | 0/21 (0%) | 0 |

Musculoskeletal and connective tissue disorders

| | | | | | | |
|---|---|---|---|---|---|---|
| Muscular weakness ^{A} † | 0/17 (0%) | 0 | 1/16 (6.25%) | 1 | 0/21 (0%) | 0 |

Neoplasms benign, malignant and unspecified (incl cysts and polyps)

| | | | | | | |
|---|---|---|---|---|---|---|
| Acute lymphocytic leukaemia recurrent ^{A} † | 0/17 (0%) | 0 | 0/16 (0%) | 0 | 1/21 (4.76%) | 1 |
| Leukaemia recurrent ^{A} † | 0/17 (0%) | 0 | 0/16 (0%) | 0 | 1/21 (4.76%) | 1 |
| Metastases to meninges ^{A} † | 1/17 (5.88%) | 1 | 0/16 (0%) | 0 | 0/21 (0%) | 0 |
| Myelodysplastic syndrome ^{A} † | 0/17 (0%) | 0 | 0/16 (0%) | 0 | 1/21 (4.76%) | 1 |

Nervous system disorders

| | | | | | | |
|---|---|---|---|---|---|---|
| Lacunar infarction ^{A} † | 0/17 (0%) | 0 | 1/16 (6.25%) | 1 | 0/21 (0%) | 0 |

Renal and urinary disorders

| | | | | | | |
|---|---|---|---|---|---|---|
| Acute kidney injury ^{A} † | 0/17 (0%) | 0 | 1/16 (6.25%) | 1 | 0/21 (0%) | 0 |
| Proteinuria ^{A} † | 1/17 (5.88%) | 1 | 0/16 (0%) | 0 | 0/21 (0%) | 0 |
| Urinary retention ^{A} † | 0/17 (0%) | 0 | 0/16 (0%) | 0 | 1/21 (4.76%) | 1 |

Respiratory, thoracic and mediastinal disorders

| | | | | | | |
|---|---|---|---|---|---|---|
| Acute respiratory failure ^{A} † | 1/17 (5.88%) | 1 | 0/16 (0%) | 0 | 0/21 (0%) | 0 |
| Dyspnoea ^{A} † | 0/17 (0%) | 0 | 1/16 (6.25%) | 1 | 3/21 (14.29%) | 3 |
| Hypoxia ^{A} † | 0/17 (0%) | 0 | 0/16 (0%) | 0 | 2/21 (9.52%) | 2 |
| Obliterative bronchiolitis ^{A} † | 1/17 (5.88%) | 1 | 0/16 (0%) | 0 | 0/21 (0%) | 0 |
| Pleural effusion ^{A} † | 0/17 (0%) | 0 | 0/16 (0%) | 0 | 1/21 (4.76%) | 1 |

Social circumstances

| | | | | | | |
|---|---|---|---|---|---|---|
| Pregnancy of partner ^{A} † | 0/17 (0%) | 0 | 0/16 (0%) | 0 | 1/21 (4.76%) | 1 |

Vascular disorders

| | | | | | | |
|---|---|---|---|---|---|---|
| Embolism ^{A} † | 0/17 (0%) | 0 | 1/16 (6.25%) | 1 | 0/21 (0%) | 0 |

†Indicates events were collected by systematic assessment.
^{A} Term from vocabulary, MedDRA 24.1

TABLE 16
Other Adverse Events
Frequency Threshold Above Which Other Events are Reported: 5%

| | Cohort 1 200 mg QD | | Cohort 2 200 mg BID | | Cohort 3 400 mg QD | |
|---|---|---|---|---|---|---|
| | Affected/At | # | Affected/At | # | Affected/At | # |
| | Risk (%) | Events | Risk (%) | Events | Risk (%) | Events |
| Total | 17/17 (100%) | | 16/16 (100%) | | 19/21 (90.48%) | |

Blood and lymphatic system disorders

| | | | | | | |
|---|---|---|---|---|---|---|
| Anaemia ^{A} † | 5/17 (29.41%) | 14 | 4/16 (25%) | 8 | 0/21 (0%) | 0 |
| Blood loss anaemia ^{A} † | 1/17 (5.88%) | 1 | 0/16 (0%) | 0 | 0/21 (0%) | 0 |
| Leukocytosis ^{A} † | 1/17 (5.88%) | 1 | 0/16 (0%) | 0 | 1/21 (4.76%) | 1 |
| Thrombocytopenia ^{A} † | 0/17 (0%) | 0 | 1/16 (6.25%) | 1 | 1/21 (4.76%) | 1 |
| Thrombocytosis ^{A} † | 1/17 (5.88%) | 1 | 0/16 (0%) | 0 | 0/21 (0%) | 0 |

Cardiac disorders

| | | | | | | |
|---|---|---|---|---|---|---|
| Palpitations ^{A} † | 1/17 (5.88%) | 1 | 0/16 (0%) | 0 | 1/21 (4.76%) | 1 |
| Pericardial effusion ^{A} † | 1/17 (5.88%) | 1 | 0/16 (0%) | 0 | 0/21 (0%) | 0 |
| Tachycardia ^{A} † | 0/17 (0%) | 0 | 0/16 (0%) | 0 | 2/21 (9.52%) | 2 |

Ear and labyrinth disorders

| | | | | | | |
|---|---|---|---|---|---|---|
| Vertigo ^{A} † | 2/17 (11.76%) | 2 | 0/16 (0%) | 0 | 0/21 (0%) | 0 |

Endocrine disorders

| | | | | | | |
|---|---|---|---|---|---|---|
| Adrenal insufficiency ^{A} † | 1/17 (5.88%) | 2 | 0/16 (0%) | 0 | 0/21 (0%) | 0 |
| Hyperthyroidism ^{A} † | 0/17 (0%) | 0 | 1/16 (6.25%) | 1 | 1/21 (4.76%) | 1 |
| Hypogonadism ^{A} † | 1/17 (5.88%) | 1 | 0/16 (0%) | 0 | 0/21 (0%) | 0 |
| Hypothyroidism ^{A} † | 1/17 (5.88%) | 2 | 1/16 (6.25%) | 2 | 3/21 (14.29%) | 4 |

Eye disorders

| | | | | | | |
|---|---|---|---|---|---|---|
| Blepharitis ^{A} † | 1/17 (5.88%) | 1 | 0/16 (0%) | 0 | 1/21 (4.76%) | 2 |
| Conjunctival haemorrhage ^{A} † | 0/17 (0%) | 0 | 1/16 (6.25%) | 1 | 1/21 (4.76%) | 1 |
| Dry eye ^{A} † | 2/17 (11.76%) | 3 | 4/16 (25%) | 4 | 2/21 (9.52%) | 2 |
| Eye irritation ^{A} † | 1/17 (5.88%) | 1 | 0/16 (0%) | 0 | 0/21 (0%) | 0 |
| Eye pain ^{A} † | 0/17 (0%) | 0 | 2/16 (12.5%) | 4 | 0/21 (0%) | 0 |
| Eye pruritus ^{A} † | 0/17 (0%) | 0 | 1/16 (6.25%) | 1 | 0/21 (0%) | 0 |
| Eyelid margin crusting ^{A} † | 1/17 (5.88%) | 1 | 0/16 (0%) | 0 | 0/21 (0%) | 0 |
| Glaucoma ^{A} † | 0/17 (0%) | 0 | 1/16 (6.25%) | 1 | 0/21 (0%) | 0 |
| Lacrimation increased ^{A} † | 0/17 (0%) | 0 | 1/16 (6.25%) | 1 | 0/21 (0%) | 0 |
| Macular degeneration ^{A} † | 0/17 (0%) | 0 | 1/16 (6.25%) | 1 | 0/21 (0%) | 0 |
| Photophobia ^{A} † | 0/17 (0%) | 0 | 1/16 (6.25%) | 1 | 2/21 (9.52%) | 2 |
| Ulcerative keratitis ^{A} † | 0/17 (0%) | 0 | 1/16 (6.25%) | 1 | 0/21 (0%) | 0 |
| Vision blurred ^{A} † | 0/17 (0%) | 0 | 2/16 (12.5%) | 2 | 4/21 (19.05%) | 4 |

Gastrointestinal disorders

| | | | | | | |
|---|---|---|---|---|---|---|
| Abdominal distension ^{A} † | 1/17 (5.88%) | 1 | 0/16 (0%) | 0 | 1/21 (4.76%) | 1 |
| Abdominal pain ^{A} † | 2/17 (11.76%) | 2 | 2/16 (12.5%) | 4 | 4/21 (19.05%) | 6 |
| Abdominal pain upper ^{A} † | 1/17 (5.88%) | 1 | 1/16 (6.25%) | 1 | 0/21 (0%) | 0 |
| Anal incontinence ^{A} † | 1/17 (5.88%) | 2 | 0/16 (0%) | 0 | 0/21 (0%) | 0 |
| Constipation ^{A} † | 1/17 (5.88%) | 1 | 1/16 (6.25%) | 1 | 3/21 (14.29%) | 4 |
| Diarrhoea ^{A} † | 6/17 (35.29%) | 9 | 5/16 (31.25%) | 10 | 7/21 (33.33%) | 11 |
| Dyspepsia ^{A} † | 0/17 (0%) | 0 | 2/16 (12.5%) | 2 | 1/21 (4.76%) | 1 |
| Dysphagia ^{A} † | 1/17 (5.88%) | 1 | 3/16 (18.75%) | 4 | 2/21 (9.52%) | 3 |
| Enamel anomaly ^{A} † | 0/17 (0%) | 0 | 1/16 (6.25%) | 1 | 0/21 (0%) | 0 |
| Flatulence ^{A} † | 0/17 (0%) | 0 | 1/16 (6.25%) | 2 | 1/21 (4.76%) | 1 |
| Gastric ulcer ^{A} † | 0/17 (0%) | 0 | 1/16 (6.25%) | 1 | 0/21 (0%) | 0 |
| Gastrooesophageal reflux disease ^{A} † | 2/17 (11.76%) | 2 | 1/16 (6.25%) | 1 | 2/21 (9.52%) | 2 |
| Nausea ^{A} † | 6/17 (35.29%) | 10 | 4/16 (25%) | 6 | 9/21 (42.86%) | 12 |
| Oesophageal ulcer ^{A} † | 0/17 (0%) | 0 | 1/16 (6.25%) | 1 | 0/21 (0%) | 0 |
| Stomatitis ^{A} † | 1/17 (5.88%) | 2 | 1/16 (6.25%) | 1 | 1/21 (4.76%) | 1 |
| Vomiting ^{A} † | 2/17 (11.76%) | 4 | 3/16 (18.75%) | 3 | 4/21 (19.05%) | 4 |

General disorders

| | | | | | | |
|---|---|---|---|---|---|---|
| Asthenia ^{A} † | 0/17 (0%) | 0 | 1/16 (6.25%) | 1 | 0/21 (0%) | 0 |
| Catheter site haemorrhage ^{A} † | 1/17 (5.88%) | 1 | 0/16 (0%) | 0 | 0/21 (0%) | 0 |
| Chills ^{A} † | 1/17 (5.88%) | 1 | 1/16 (6.25%) | 1 | 0/21 (0%) | 0 |
| Fatigue ^{A} † | 6/17 (35.29%) | 7 | 3/16 (18.75%) | 7 | 10/21 (47.62%) | 14 |
| Generalised oedema ^{A} † | 1/17 (5.88%) | 1 | 0/16 (0%) | 0 | 1/21 (4.76%) | 1 |
| Hypothermia ^{A} † | 0/17 (0%) | 0 | 1/16 (6.25%) | 1 | 0/21 (0%) | 0 |
| Impaired healing ^{A} † | 1/17 (5.88%) | 1 | 0/16 (0%) | 0 | 0/21 (0%) | 0 |
| Influenza like illness ^{A} † | 1/17 (5.88%) | 1 | 1/16 (6.25%) | 2 | 0/21 (0%) | 0 |
| Non-cardiac chest pain ^{A} † | 0/17 (0%) | 0 | 3/16 (18.75%) | 3 | 1/21 (4.76%) | 1 |
| Oedema ^{A} † | 0/17 (0%) | 0 | 1/16 (6.25%) | 1 | 0/21 (0%) | 0 |
| Oedema peripheral ^{A} † | 3/17 (17.65%) | 3 | 4/16 (25%) | 5 | 6/21 (28.57%) | 6 |
| Pain ^{A} † | 3/17 (17.65%) | 3 | 0/16 (0%) | 0 | 1/21 (4.76%) | 1 |
| Pyrexia ^{A} † | 1/17 (5.88%) | 3 | 3/16 (18.75%) | 6 | 3/21 (14.29%) | 3 |

Immune system disorders

| | | | | | | |
|---|---|---|---|---|---|---|
| Contrast media allergy ^{A} † | 0/17 (0%) | 0 | 2/16 (12.5%) | 2 | 0/21 (0%) | 0 |
| Graft versus host disease in | 1/17 (5.88%) | 1 | 0/16 (0%) | 0 | 0/21 (0%) | 0 |
| gastrointestinal tract ^{A} † | | | | | | |

Infections and infestations

| | | | | | | |
|---|---|---|---|---|---|---|
| Bronchitis ^{A} † | 1/17 (5.88%) | 1 | 0/16 (0%) | 0 | 1/21 (4.76%) | 1 |
| Conjunctivitis ^{A} † | 0/17 (0%) | 0 | 1/16 (6.25%) | 1 | 3/21 (14.29%) | 3 |
| Corneal infection ^{A} † | 0/17 (0%) | 0 | 1/16 (6.25%) | 2 | 0/21 (0%) | 0 |
| Epstein-Barr virus infection ^{A} † | 1/17 (5.88%) | 1 | 0/16 (0%) | 0 | 0/21 (0%) | 0 |
| Fungal infection ^{A} † | 1/17 (5.88%) | 1 | 0/16 (0%) | 0 | 0/21 (0%) | 0 |
| Genital infection fungal ^{A} † | 0/17 (0%) | 0 | 1/16 (6.25%) | 1 | 0/21 (0%) | 0 |
| Hordeolum ^{A} † | 1/17 (5.88%) | 1 | 0/16 (0%) | 0 | 1/21 (4.76%) | 1 |
| Influenza ^{A} † | 2/17 (11.76%) | 2 | 1/16 (6.25%) | 2 | 2/21 (9.52%) | 2 |
| Oral candidiasis ^{A} † | 2/17 (11.76%) | 3 | 1/16 (6.25%) | 1 | 0/21 (0%) | 0 |
| Pneumonia ^{A} † | 1/17 (5.88%) | 2 | 3/16 (18.75%) | 3 | 0/21 (0%) | 0 |
| Pneumonia respiratory syncytial viral ^{A} † | 0/17 (0%) | 0 | 1/16 (6.25%) | 1 | 0/21 (0%) | 0 |
| Pseudomonas infection ^{A} † | 1/17 (5.88%) | 1 | 1/16 (6.25%) | 2 | 0/21 (0%) | 0 |
| Rash pustular ^{A} † | 0/17 (0%) | 0 | 1/16 (6.25%) | 1 | 0/21 (0%) | 0 |
| Respiratory syncytial virus infection ^{A} † | 0/17 (0%) | 0 | 1/16 (6.25%) | 1 | 2/21 (9.52%) | 3 |
| Respiratory tract infection ^{A} † | 1/17 (5.88%) | 1 | 0/16 (0%) | 0 | 0/21 (0%) | 0 |
| Respiratory tract infection fungal ^{A} † | 1/17 (5.88%) | 1 | 0/16 (0%) | 0 | 0/21 (0%) | 0 |
| Sinusitis ^{A} † | 1/17 (5.88%) | 1 | 2/16 (12.5%) | 4 | 1/21 (4.76%) | 1 |
| Tinea pedis ^{A} † | 0/17 (0%) | 0 | 1/16 (6.25%) | 1 | 0/21 (0%) | 0 |
| Upper respiratory tract infection ^{A} † | 9/17 (52.94%) | 16 | 9/16 (56.25%) | 13 | 7/21 (33.33%) | 9 |
| Urinary tract infection ^{A} † | 0/17 (0%) | 0 | 2/16 (12.5%) | 2 | 0/21 (0%) | 0 |

Injury, poisoning and procedural complications

| | | | | | | |
|---|---|---|---|---|---|---|
| Contusion ^{A} † | 2/17 (11.76%) | 2 | 2/16 (12.5%) | 2 | 3/21 (14.29%) | 4 |
| Corneal abrasion ^{A} † | 1/17 (5.88%) | 1 | 0/16 (0%) | 0 | 0/21 (0%) | 0 |
| Fall ^{A} † | 2/17 (11.76%) | 2 | 1/16 (6.25%) | 1 | 1/21 (4.76%) | 1 |
| Ligament sprain ^{A} † | 1/17 (5.88%) | 1 | 0/16 (0%) | 0 | 0/21 (0%) | 0 |
| Limb injury ^{A} † | 1/17 (5.88%) | 1 | 0/16 (0%) | 0 | 1/21 (4.76%) | 1 |
| Overdose ^{A} † | 1/17 (5.88%) | 1 | 0/16 (0%) | 0 | 0/21 (0%) | 0 |
| Skin abrasion ^{A} † | 0/17 (0%) | 0 | 0/16 (0%) | 0 | 2/21 (9.52%) | 2 |
| Spinal compression fracture ^{A} † | 1/17 (5.88%) | 1 | 0/16 (0%) | 0 | 0/21 (0%) | 0 |
| Tooth fracture ^{A} † | 0/17 (0%) | 0 | 1/16 (6.25%) | 1 | 0/21 (0%) | 0 |
| Transfusion reaction ^{A} † | 1/17 (5.88%) | 1 | 0/16 (0%) | 0 | 0/21 (0%) | 0 |
| Vascular access complication ^{A} † | 0/17 (0%) | 0 | 1/16 (6.25%) | 1 | 0/21 (0%) | 0 |

Investigations

| | | | | | | |
|---|---|---|---|---|---|---|
| Alanine aminotransferase increased ^{A} † | 6/17 (35.29%) | 12 | 3/16 (18.75%) | 4 | 2/21 (9.52%) | 2 |
| Aspartate aminotransferase increased ^{A} † | 5/17 (29.41%) | 8 | 2/16 (12.5%) | 2 | 1/21 (4.76%) | 1 |
| Blood alkaline phosphatase increased ^{A} † | 4/17 (23.53%) | 6 | 1/16 (6.25%) | 1 | 0/21 (0%) | 0 |
| Blood chloride decreased ^{A} † | 0/17 (0%) | 0 | 1/16 (6.25%) | 1 | 0/21 (0%) | 0 |
| Blood cholesterol increased ^{A} † | 1/17 (5.88%) | 2 | 0/16 (0%) | 0 | 1/21 (4.76%) | 1 |
| Blood creatine increased ^{A} † | 0/17 (0%) | 0 | 1/16 (6.25%) | 1 | 0/21 (0%) | 0 |
| Blood creatine phosphokinase increased ^{A} † | 1/17 (5.88%) | 1 | 0/16 (0%) | 0 | 0/21 (0%) | 0 |
| Blood creatinine increased ^{A} † | 1/17 (5.88%) | 1 | 1/16 (6.25%) | 1 | 2/21 (9.52%) | 2 |
| Bronchoscopy ^{A} † | 1/17 (5.88%) | 1 | 0/16 (0%) | 0 | 0/21 (0%) | 0 |
| Gamma-glutamyltransferase increased ^{A} † | 4/17 (23.53%) | 9 | 4/16 (25%) | 4 | 0/21 (0%) | 0 |
| Glucose urine present ^{A} † | 2/17 (11.76%) | 3 | 0/16 (0%) | 0 | 1/21 (4.76%) | 3 |
| Neutrophil count decreased ^{A} † | 1/17 (5.88%) | 1 | 0/16 (0%) | 0 | 1/21 (4.76%) | 1 |
| Platelet count decreased ^{A} † | 1/17 (5.88%) | 3 | 0/16 (0%) | 0 | 0/21 (0%) | 0 |
| Red blood cells urine positive ^{A} † | 0/17 (0%) | 0 | 1/16 (6.25%) | 1 | 0/21 (0%) | 0 |
| Urine output decreased ^{A} † | 1/17 (5.88%) | 1 | 1/16 (6.25%) | 1 | 0/21 (0%) | 0 |
| Weight decreased ^{A} † | 1/17 (5.88%) | 1 | 0/16 (0%) | 0 | 2/21 (9.52%) | 4 |
| Weight increased ^{A} † | 3/17 (17.65%) | 9 | 1/16 (6.25%) | 2 | 1/21 (4.76%) | 4 |
| White blood cell count decreased ^{A} † | 1/17 (5.88%) | 1 | 0/16 (0%) | 0 | 1/21 (4.76%) | 2 |

Metabolism and nutrition disorders

| | | | | | | |
|---|---|---|---|---|---|---|
| Decreased appetite ^{A} † | 2/17 (11.76%) | 4 | 2/16 (12.5%) | 2 | 4/21 (19.05%) | 4 |
| Dehydration ^{A} † | 2/17 (11.76%) | 2 | 3/16 (18.75%) | 4 | 2/21 (9.52%) | 2 |
| Hyperglycemia ^{A} † | 2/17 (11.76%) | 2 | 0/16 (0%) | 0 | 3/21 (14.29%) | 8 |
| Hyperkalaemia ^{A} † | 3/17 (17.65%) | 7 | 2/16 (12.5%) | 2 | 3/21 (14.29%) | 4 |
| Hypertriglyceridaemia ^{A} † | 0/17 (0%) | 0 | 1/16 (6.25%) | 1 | 0/21 (0%) | 0 |
| Hyperuricaemia ^{A} † | 1/17 (5.88%) | 6 | 1/16 (6.25%) | 2 | 3/21 (14.29%) | 5 |
| Hypocalcaemia ^{A} † | 0/17 (0%) | 0 | 1/16 (6.25%) | 4 | 1/21 (4.76%) | 1 |
| Hypokalaemia ^{A} † | 1/17 (5.88%) | 1 | 2/16 (12.5%) | 3 | 0/21 (0%) | 0 |
| Hypomagnesaemia ^{A} † | 1/17 (5.88%) | 1 | 1/16 (6.25%) | 1 | 1/21 (4.76%) | 1 |
| Hyponatraemia ^{A} † | 1/17 (5.88%) | 1 | 0/16 (0%) | 0 | 2/21 (9.52%) | 2 |
| Hypophosphataemia ^{A} † | 1/17 (5.88%) | 1 | 1/16 (6.25%) | 1 | 1/21 (4.76%) | 1 |
| Obesity ^{A} † | 2/17 (11.76%) | 2 | 0/16 (0%) | 0 | 0/21 (0%) | 0 |
| Type 2 diabetes mellitus ^{A} † | 1/17 (5.88%) | 1 | 0/16 (0%) | 0 | 0/21 (0%) | 0 |

Musculoskeletal and connective tissue disorders

| | | | | | | |
|---|---|---|---|---|---|---|
| Arthralgia ^{A} † | 1/17 (5.88%) | 1 | 1/16 (6.25%) | 1 | 5/21 (23.81%) | 6 |
| Back pain ^{A} † | 0/17 (0%) | 0 | 2/16 (12.5%) | 2 | 1/21 (4.76%) | 1 |
| Joint range of motion decreased ^{A} † | 0/17 (0%) | 0 | 1/16 (6.25%) | 1 | 1/21 (4.76%) | 1 |
| Muscle spasms ^{A} † | 1/17 (5.88%) | 1 | 0/16 (0%) | 0 | 6/21 (28.57%) | 6 |
| Muscular weakness ^{A} † | 4/17 (23.53%) | 6 | 1/16 (6.25%) | 1 | 2/21 (9.52%) | 2 |
| Musculoskeletal chest pain ^{A} † | 1/17 (5.88%) | 1 | 0/16 (0%) | 0 | 1/21 (4.76%) | 1 |
| Myalgia ^{A} † | 1/17 (5.88%) | 2 | 1/16 (6.25%) | 1 | 3/21 (14.29%) | 3 |
| Neck pain ^{A} † | 1/17 (5.88%) | 1 | 0/16 (0%) | 0 | 0/21 (0%) | 0 |
| Osteonecrosis ^{A} † | 1/17 (5.88%) | 1 | 0/16 (0%) | 0 | 0/21 (0%) | 0 |
| Osteopenia ^{A} † | 0/17 (0%) | 0 | 1/16 (6.25%) | 1 | 0/21 (0%) | 0 |
| Osteoporosis ^{A} † | 0/17 (0%) | 0 | 1/16 (6.25%) | 1 | 0/21 (0%) | 0 |
| Pain in extremity ^{A} † | 2/17 (11.76%) | 2 | 2/16 (12.5%) | 3 | 4/21 (19.05%) | 5 |
| Tendonitis ^{A} † | 0/17 (0%) | 0 | 1/16 (6.25%) | 1 | 0/21 (0%) | 0 |
| Thoracic spinal stenosis ^{A} † | 1/17 (5.88%) | 1 | 0/16 (0%) | 0 | 0/21 (0%) | 0 |

Neoplasms benign, malignant and unspecified (incl cysts and polyps)

| | | | | | | |
|---|---|---|---|---|---|---|
| Basal cell carcinoma ^{A} † | 0/17 (0%) | 0 | 1/16 (6.25%) | 1 | 0/21 (0%) | 0 |
| Hodgkin's disease recurrent ^{A} † | 1/17 (5.88%) | 1 | 0/16 (0%) | 0 | 0/21 (0%) | 0 |
| Leukaemia recurrent ^{A} † | 1/17 (5.88%) | 1 | 0/16 (0%) | 0 | 0/21 (0%) | 0 |
| Skin papilloma ^{A} † | 1/17 (5.88%) | 1 | 0/16 (0%) | 0 | 0/21 (0%) | 0 |
| Squamous cell carcinoma of skin ^{A} † | 0/17 (0%) | 0 | 2/16 (12.5%) | 2 | 0/21 (0%) | 0 |

Nervous system disorders

| | | | | | | |
|---|---|---|---|---|---|---|
| Balance disorder ^{A} † | 0/17 (0%) | 0 | 1/16 (6.25%) | 1 | 0/21 (0%) | 0 |
| Dizziness ^{A} † | 0/17 (0%) | 0 | 1/16 (6.25%) | 4 | 2/21 (9.52%) | 2 |
| Epidural lipomatosis ^{A} † | 1/17 (5.88%) | 1 | 0/16 (0%) | 0 | 0/21 (0%) | 0 |
| Headache ^{A} † | 4/17 (23.53%) | 4 | 3/16 (18.75%) | 3 | 6/21 (28.57%) | 8 |
| Hypoaesthesia ^{A} † | 1/17 (5.88%) | 2 | 0/16 (0%) | 0 | 1/21 (4.76%) | 1 |
| Migraine ^{A} † | 0/17 (0%) | 0 | 1/16 (6.25%) | 1 | 0/21 (0%) | 0 |
| Muscle spasticity ^{A} † | 1/17 (5.88%) | 1 | 0/16 (0%) | 0 | 1/21 (4.76%) | 1 |
| Neuropathy peripheral ^{A} † | 0/17 (0%) | 0 | 1/16 (6.25%) | 1 | 1/21 (4.76%) | 2 |
| Paraesthesia ^{A} † | 0/17 (0%) | 0 | 1/16 (6.25%) | 1 | 1/21 (4.76%) | 1 |
| Peripheral sensory neuropathy ^{A} † | 0/17 (0%) | 0 | 1/16 (6.25%) | 1 | 0/21 (0%) | 0 |
| Presyncope ^{A} † | 0/17 (0%) | 0 | 3/16 (18.75%) | 3 | 0/21 (0%) | 0 |
| Restless legs syndrome ^{A} † | 0/17 (0%) | 0 | 1/16 (6.25%) | 1 | 0/21 (0%) | 0 |
| Tremor ^{A} † | 2/17 (11.76%) | 3 | 1/16 (6.25%) | 2 | 3/21 (14.29%) | 4 |

Psychiatric disorders

| | | | | | | |
|---|---|---|---|---|---|---|
| Anxiety ^{A} † | 1/17 (5.88%) | 1 | 2/16 (12.5%) | 3 | 3/21 (14.29%) | 3 |
| Depression ^{A} † | 0/17 (0%) | 0 | 4/16 (25%) | 4 | 3/21 (14.29%) | 3 |
| Insomnia ^{A} † | 0/17 (0%) | 0 | 0/16 (0%) | 0 | 3/21 (14.29%) | 3 |
| Irritability ^{A} † | 1/17 (5.88%) | 1 | 0/16 (0%) | 0 | 0/21 (0%) | 0 |

Renal and urinary disorders

| | | | | | | |
|---|---|---|---|---|---|---|
| Acute kidney injury ^{A} † | 1/17 (5.88%) | 4 | 2/16 (12.5%) | 2 | 2/21 (9.52%) | 6 |
| Chronic kidney disease ^{A} † | 1/17 (5.88%) | 1 | 0/16 (0%) | 0 | 0/21 (0%) | 0 |
| Haematuria ^{A} † | 1/17 (5.88%) | 1 | 1/16 (6.25%) | 1 | 1/21 (4.76%) | 2 |
| Nephrotic syndrome ^{A} † | 1/17 (5.88%) | 1 | 1/16 (6.25%) | 1 | 0/21 (0%) | 0 |
| Nocturia ^{A} † | 1/17 (5.88%) | 1 | 0/16 (0%) | 0 | 0/21 (0%) | 0 |
| Pollakiuria ^{A} † | 1/17 (5.88%) | 1 | 0/16 (0%) | 0 | 0/21 (0%) | 0 |
| Proteinuria ^{A} † | 2/17 (11.76%) | 2 | 1/16 (6.25%) | 1 | 0/21 (0%) | 0 |
| Renal failure ^{A} † | 0/17 (0%) | 0 | 1/16 (6.25%) | 1 | 0/21 (0%) | 0 |
| Urinary retention ^{A} † | 0/17 (0%) | 0 | 1/16 (6.25%) | 2 | 0/21 (0%) | 0 |
| Urinary tract pain ^{A} † | 0/17 (0%) | 0 | 1/16 (6.25%) | 1 | 1/21 (4.76%) | 1 |

Reproductive system and breast disorders

| | | | | | | |
|---|---|---|---|---|---|---|
| Benign prostatic hyperplasia ^{A} † | 1/17 (5.88%) | 1 | 0/16 (0%) | 0 | 0/21 (0%) | 0 |
| Erectile dysfunction ^{A} † | 2/17 (11.76%) | 4 | 0/16 (0%) | 0 | 0/21 (0%) | 0 |
| Gynaecomastia ^{A} † | 0/17 (0%) | 0 | 1/16 (6.25%) | 1 | 0/21 (0%) | 0 |
| Testicular pain ^{A} † | 1/17 (5.88%) | 1 | 0/16 (0%) | 0 | 0/21 (0%) | 0 |
| Testicular swelling ^{A} † | 1/17 (5.88%) | 1 | 0/16 (0%) | 0 | 0/21 (0%) | 0 |

Respiratory, thoracic and mediastinal disorders

| | | | | | | |
|---|---|---|---|---|---|---|
| Acute respiratory distress syndrome ^{A} † | 1/17 (5.88%) | 1 | 0/16 (0%) | 0 | 0/21 (0%) | 0 |
| Atelectasis ^{A} † | 1/17 (5.88%) | 1 | 0/16 (0%) | 0 | 0/21 (0%) | 0 |
| Bronchial secretion retention ^{A} † | 0/17 (0%) | 0 | 1/16 (6.25%) | 1 | 0/21 (0%) | 0 |
| Cough ^{A} † | 1/17 (5.88%) | 1 | 4/16 (25%) | 5 | 7/21 (33.33%) | 9 |
| Dyspnoea ^{A} † | 2/17 (11.76%) | 2 | 5/16 (31.25%) | 7 | 6/21 (28.57%) | 6 |
| Dyspnoea exertional ^{A} † | 1/17 (5.88%) | 1 | 0/16 (0%) | 0 | 0/21 (0%) | 0 |
| Hypoxia ^{A} † | 1/17 (5.88%) | 1 | 2/16 (12.5%) | 5 | 3/21 (14.29%) | 3 |
| Nasal congestion ^{A} † | 3/17 (17.65%) | 3 | 0/16 (0%) | 0 | 1/21 (4.76%) | 1 |
| Oropharyngeal pain ^{A} † | 1/17 (5.88%) | 1 | 0/16 (0%) | 0 | 1/21 (4.76%) | 1 |
| Orthopnoea ^{A} † | 1/17 (5.88%) | 1 | 0/16 (0%) | 0 | 0/21 (0%) | 0 |
| Pleural effusion ^{A} † | 1/17 (5.88%) | 1 | 0/16 (0%) | 0 | 1/21 (4.76%) | 1 |
| Productive cough ^{A} † | 2/17 (11.76%) | 2 | 2/16 (12.5%) | 7 | 1/21 (4.76%) | 2 |
| Pulmonary mass ^{A} † | 0/17 (0%) | 0 | 1/16 (6.25%) | 1 | 1/21 (4.76%) | 1 |
| Sinus pain ^{A} † | 2/17 (11.76%) | 2 | 0/16 (0%) | 0 | 0/21 (0%) | 0 |
| Upper-airway cough syndrome ^{A} † | 0/17 (0%) | 0 | 2/16 (12.5%) | 2 | 1/21 (4.76%) | 1 |

Skin and subcutaneous tissue disorders

| | | | | | | |
|---|---|---|---|---|---|---|
| Actinic keratosis ^{A} † | 0/17 (0%) | 0 | 1/16 (6.25%) | 1 | 1/21 (4.76%) | 1 |
| Blister ^{A} † | 0/17 (0%) | 0 | 2/16 (12.5%) | 3 | 1/21 (4.76%) | 1 |
| Drug eruption ^{A} † | 0/17 (0%) | 0 | 1/16 (6.25%) | 1 | 0/21 (0%) | 0 |
| Dry skin ^{A} † | 1/17 (5.88%) | 1 | 0/16 (0%) | 0 | 1/21 (4.76%) | 1 |
| Hair growth abnormal ^{A} † | 1/17 (5.88%) | 1 | 0/16 (0%) | 0 | 0/21 (0%) | 0 |
| Hyperhidrosis ^{A} † | 0/17 (0%) | 0 | 1/16 (6.25%) | 1 | 1/21 (4.76%) | 1 |
| Nail disorder ^{A} † | 1/17 (5.88%) | 1 | 0/16 (0%) | 0 | 0/21 (0%) | 0 |
| Pruritus ^{A} † | 1/17 (5.88%) | 1 | 2/16 (12.5%) | 4 | 0/21 (0%) | 0 |
| Rash ^{A} † | 1/17 (5.88%) | 2 | 1/16 (6.25%) | 1 | 1/21 (4.76%) | 1 |
| Rash erythematous ^{A} † | 0/17 (0%) | 0 | 1/16 (6.25%) | 1 | 0/21 (0%) | 0 |
| Rash maculo-papular ^{A} † | 2/17 (11.76%) | 2 | 0/16 (0%) | 0 | 0/21 (0%) | 0 |
| Rosacea ^{A} † | 0/17 (0%) | 0 | 1/16 (6.25%) | 1 | 0/21 (0%) | 0 |
| Skin lesion ^{A} † | 0/17 (0%) | 0 | 1/16 (6.25%) | 1 | 0/21 (0%) | 0 |

Vascular disorders

| | | | | | | |
|---|---|---|---|---|---|---|
| Embolism ^{A} † | 1/17 (5.88%) | 1 | 0/16 (0%) | 0 | 1/21 (4.76%) | 1 |
| Flushing ^{A} † | 0/17 (0%) | 0 | 1/16 (6.25%) | 1 | 0/21 (0%) | 0 |
| Hot flush ^{A} † | 1/17 (5.88%) | 1 | 0/16 (0%) | 0 | 0/21 (0%) | 0 |
| Hypertension ^{A} † | 5/17 (29.41%) | 8 | 2/16 (12.5%) | 5 | 4/21 (19.05%) | 6 |
| Peripheral coldness ^{A} † | 1/17 (5.88%) | 1 | 0/16 (0%) | 0 | 0/21 (0%) | 0 |
| Phlebitis ^{A} † | 0/17 (0%) | 0 | 1/16 (6.25%) | 1 | 0/21 (0%) | 0 |
| Superior vena cava syndrome ^{A} † | 0/17 (0%) | 0 | 1/16 (6.25%) | 2 | 1/21 (4.76%) | 0 |

† Indicates events were collected by systematic assessment.
^{A} Term from vocabulary, MedDRA 24.1

Number of Participants With Treatment-emergent Adverse Events (TEAEs), and Treatment-emergent Serious Adverse Events (TESAEs). An adverse event (AE) was defined as any untoward medical occurrence in a participant who received study drug and did not necessarily have to have a causal relationship with the treatment. Serious adverse events (SAEs) was any untoward medical occurrence that at any dose: resulted in death, was life-threatening, required inpatient hospitalization or prolongation of existing hospitalization, resulted in persistent or significant disability/incapacity, was a congenital anomaly/birth defect, was a medically important event. TEAEs were defined as AEs that developed, worsened or became serious during the TEAE period (defined as the time from the first dose of study treatment up to 28 days after the last dose of study drug). TEAEs included both SAEs and non-SAEs. Timeframe: From first dose of study treatment up to 28 days after the last dose of study drug (maximum duration: up to 64.2 months). Analysis was performed on safety population which included all participants who received at least 1 dose of study medication. Data is shown in the table below.

TABLE 17
Number of Participants With Treatment-emergent Adverse Events
(TEAEs), and Treatment-emergent Serious Adverse Events (TESAEs)

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Number of Participants | 17 | 16 | 21 |
| With Treatment-emergent | | | |
| Adverse Events (TEAEs), | | | |
| and Treatment-emergent | | | |
| Serious Adverse Events | | | |
| (TESAEs) | | | |
| Measure Type: Count of | | | |
| Participants | | | |
| Unit of measure = | | | |
| participants | | | |
| TEAE | 17 (100%) | 16 (100%) | 20 (95%) |
| TESAE | 5 (29%) | 6 (38%) | 13 (62%) |

### PD Analyses

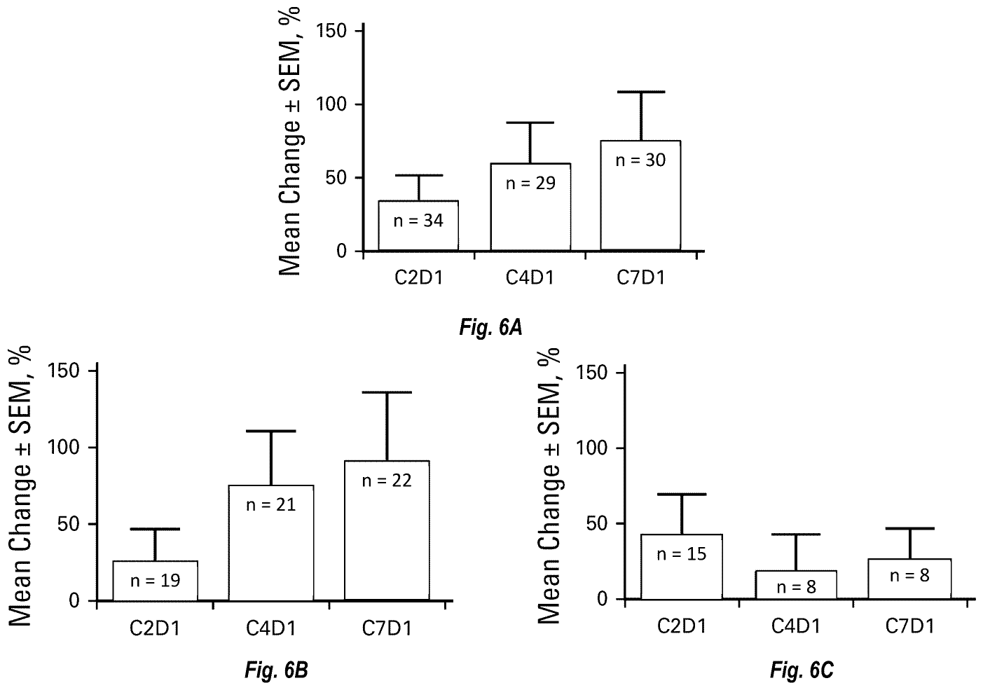

In exploratory PD analyses of peripheral blood mononuclear cells across cohorts, the percentage of CD41 Tregs demonstrated an increasing trend early on by cycle 2 day 1 of belumosudil treatment. A simultaneous decrease in Th17 cells was also observed. The Th17 cells continued to decrease through C4D1 and C6D25. The percentage of CD41 Tregs continued to increase through C4D1 and C7D1, as shown in FIGS. 6A-6C. Because of the small sample size, correlative data with steroid dose were limited for any statistical analysis.

This study was the first to evaluate belumosudil treatment in human patients with cGVHD. All phenotypes of cGVHD, without requirements for inflammatory or fibrotic manifestations, were included. Patients with advanced multiorgan cGVHD treated with belumosudil achieved an ORR of 65%, with QOL improvements, corticosteroid dose reductions, and limited toxicity. With relatively small sample sizes, there was no difference in the ORR across cohorts.

Belumosudil achieved response rates that were meaningful and consistent across subgroups, including patients with severe cGVHD, patients who had received ≥2 prior systemic lines of therapy, patients who were refractory to their last lines of therapy before enrollment, and patients with ≥4 organs involved. The ORR among patients with nonsevere cGVHD was 83%, suggesting that further studies of how belumosudil may benefit patients earlier in their disease are indicated. All responses at the patient level were PR; no CR was achieved. However, given the severity and extent of fibrotic cGVHD manifestations in this patient population, achieving CR in all organs was not expected, as some advanced fibrotic changes in the eyes, mouth, lungs, or joints and/or fascia can be irreversible. CR was observed in all organs except the lungs, where PR was achieved.

Belumosudil response kinetics suggest that most responders achieved responses rapidly within 8 weeks after receiving belumosudil. Belumosudil was well-tolerated, with a median DOR of 35 weeks across all responders. The ability to stay on therapy is dependent on the safety and long-term tolerability profile of the intervention. The median treatment duration was 8 months (range, 1-39 months). Twenty-eight percent of patients remained on belumosudil for >18 months. There was no reported CMV infection or reactivation, despite 57% of patients being CMV seropositive. The incidence of TEAEs and grade≥3TEAEs was similar across cohorts. The combination of well-tolerated therapy and efficacy in inducing responses translated into a 2-year OS rate of 82%, a median TTNT of 14 months, and FFS rates of 76% and 47% at 6 and 12 months, respectively.

In a prospective study conducted by the cGVHD Consortium, the 12-month FFS rate with response (CR/PR) after first-line therapy was 12% to 15%. (Martin P J, Storer B E, Inamoto Y, et al: An endpoint associated with clinical benefit after initial treatment for chronic graft-versus-host disease. Blood 130:360-367, 2017) In this study (after 1-3 prior lines of therapy), the 12-month FFS rate with response was 24%.

In the study of the present example, Belumosudil therapy was associated with a corticosteroid-sparing effect. The current treatment paradigm relies on corticosteroids as the mainstay of therapy; however, the related long-term toxicities mandate the use of the lowest possible dose or discontinuation whenever possible. The use of corticosteroid therapy is tied to quality of life, as the side effect profile of corticosteroid therapy contributes to patient symptom burden. Corticosteroid dose reduction was observed across both responders and nonresponders to belumosudil. Approximately 20% of patients were able to discontinue corticosteroid therapy during belumosudil treatment. Even in the absence of an NIH-defined response, patients experienced clinical benefit, as evidenced by improvements in LSS score or reductions in corticosteroid doses.

Change from Baseline in Corticosteroids Dose. An EOT visit was performed within 3 days after the participant's last dose of study drug. Baseline value was defined as valid and last non-missing value obtained within 28 days prior to participant receiving first study medication. The mITT population was analyzed, with the ‘overall number of participants analyzed’=participants with available data for this outcome measure. Timeframe: baseline up to end of treatment (i.e., up to 64.2 months). The data is shown in the table below.

TABLE 18
Change From Baseline in Corticosteroids Dose

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number | 17 | 15 | 19 |
| of Participants | | | |
| Analyzed | | | |
| Change From | −0.110 (0.101) | −0.111 (0.147) | −0.116 (0.152) |
| Baseline in | | | |
| Corticosteroids | | | |
| Dose Mean | | | |
| (Standard | | | |
| Deviation) | | | |
| Unit of measure | | | |
| (milligrams per | | | |
| kilogram per day) | | | |

Change from Baseline in Calcineurin Inhibitor (CNI). Calcineurin inhibitors included systemic tacrolimus and cyclosporine. Number of participants who took CNI at Baseline and had reduction and discontinuation in CNI use as compared to Baseline during the study are reported in this outcome measure. EOT visit was performed within 3 days after the participant's last dose of study drug. Baseline value was defined as valid and last non-missing value obtained within 28 days prior to participant receiving first study medication. Timeframe: baseline up to end of treatment (i.e., up to 64.2 months). The data is shown in the table below.

TABLE 19
CNI

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number of | 6 | 6 | 11 |
| Participants Analyzed | | | |
| Participants with | 5 (83%) | 5 (83%) | 6 (55%) |
| reduction in CNI dose | | | |
| Participants who | 0 (0%) | 1 (17%) | 3 (27%) |
| discontinued CNI | | | |

Time to Response (TTR). Time-to-response was measured as the time (in weeks) from first dose of study drug to the time of first documentation of response. Response was defined as the subjects achieving a PR or CR at any post-baseline response assessment. Per the 2014 NIH Consensus Development Project for Clinical Trials in cGVHD criteria; CR was defined as the resolution of all manifestations in each organ or site and PR was defined as the improvement in at least 1 organ or site without progression in any other organ or site. Timeframe: from first treatment to the time of first documentation of response or data cut-off, whichever occurred first (maximum duration: up to 64.2 months). The analysis was performed on responder population. The data is shown in the table below:

TABLE 20

| | Cohort 1: | Cohort 2: | Cohort 3: |
|---|---|---|---|
| | Belumosudil | Belumosudil | Belumosudil |
| | 200 mg QD | 200 mg BID | 400 mg QD |
| Overall Number of | 11 | 11 | 12 |
| Participants Analyzed | | | |
| TTR | 8.14 | 8.14 | 8.07 |
| Median (Full Range) | (7.9 to 26.1) | (4.1 to 40.0) | (4.1 to 67.0) |
| Unit of Measure | | | |
| (weeks) | | | |

In this study, responses were achieved in patients with fibrotic manifestations in the lungs, joints and/or fascia, and eyes. These responses were observed in some cases after 24 weeks of treatment, further highlighting the need to sustain effective therapy to achieve clinical benefit, particularly in patients with difficult-to-treat disease. Because the lower belumosudil 200-mg once daily dose was equally safe and effective, it has been further compared in the study described in Example 2 against the 200-mg twice a day dose for final dose recommendation.

Change From Baseline in Overall Score on Lee cGvHD Symptom Scale at Specified Time Points. Lee cGVHD symptom scale, a patient-reported symptom scale used to measure symptom burden and has 7 subscales (Skin, Eyes and Mouth, Breathing, Eating and Digestion, Muscles and Joints, Energy, and Mental and Emotional) with ratings as follows: 0—Not at all, 1—Slightly, 2—Moderately, 3—Quite a bit, 4—Extremely, with lower values representing better outcome. Score for each subscale was normalized to a score ranged from 0 to 100, where higher score=worse symptoms. An overall score was calculated as average of these 7 subscales. EOT visit was performed within 3 days after the participant's last dose of study drug. Baseline value was defined as valid and last non-missing value obtained within 28 days prior to participant receiving first study medication. Timeframe: Baseline, Day 1 of Cycles 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67 and EOT (i.e., anytime up to 64.2 months). Analysis was performed on mITT population. Here, ‘number analyzed’=participants with available data for each specified category. Here, ‘0’ in the number analyzed field signifies that none of the participants were available for the analysis at the specified time points. The data is shown in the table below.

TABLE 21
Change From Baseline in Overall Score on Lee cGvHD
Symptom Scale at Specified Time Points

| | | Cohort 1: | Cohort 2: | Cohort 3: |
|---|---|---|---|---|
| | | Belumosudil | Belumosudil | Belumosudil |
| | | 200 mg QD | 200 mg BID | 400 mg QD |
| Overall Number of | | 17 | 16 | 21 |
| Participants Analyzed | | | | |
| Change From | | | | |
| Baseline | | | | |
| in Overall | | | | |
| Score on | | | | |
| Lee cGvHD | | | | |
| Symptom | | | | |
| Scale at | | | | |
| Specified Time | | | | |
| Points | | | | |
| Mean | | | | |
| (Standard | | | | |
| Deviation) | | | | |
| Unit of | | | | |
| measure: score | | | | |
| on a scale | | | | |
| Cycle 2, Day 1 | Number | 0 | 0 | 8 |
| | Analyzed | participants | participants | participants |
| | | — | — | −5.2 (6.3) |
| Cycle 3, Day 1 | Number | 16 | 12 | 16 |
| | Analyzed | participants | participants | participants |
| | | −3.0 (8.5) | 0.5 (7.3) | −4.3 (8.2) |
| Cycle 4, Day 1 | Number | 0 | 0 | 9 |
| | Analyzed | participants | participants | participants |
| | | — | — | −2.7 (8.2) |
| Cycle 5, Day 1 | Number | 13 | 10 | 12 |
| | Analyzed | participants | participants | participants |
| | | −3.7 (11.0) | −3.7 (7.0) | −0.5 (10.8) |
| Cycle 6, Day 1 | Number | 0 | 0 | 12 |
| | Analyzed | participants | participants | participants |
| | | — | — | −0.8 (9.3) |
| Cycle 7, Day 1 | Number | 10 | 9 | 12 |
| | Analyzed | participants | participants | participants |
| | | −4.9 (8.4) | −2.2 (8.8) | −2.0 (12.3) |
| Cycle 8, Day 1 | Number | 0 | 2 | 11 |
| | Analyzed | participants | participants | participants |
| | | — | −7.1 (10.1) | −2.9 (10.9) |
| Cycle 9, Day 1 | Number | 8 | 7 | 10 |
| | Analyzed | participants | participants | participants |
| | | −3.7 (10.7) | −4.2 (5.5) | −3.6 (10.2) |
| Cycle 10, Day 1 | Number | 0 | 4 | 9 |
| | Analyzed | participants | participants | participants |
| | | — | −2.7 (6.1) | −3.7 (9.9) |
| Cycle 11, Day 1 | Number | 7 | 6 | 9 |
| | Analyzed | participants | participants | participants |
| | | −3.4 (11.5) | −5.5 (6.0) | −4.5 (6.1) |
| Cycle 12, Day 1 | Number | 2 | 5 | 8 |
| | Analyzed | participants | participants | participants |
| | | −3.9 (8.1) | −7.6 (7.7) | −2.8 (6.0) |
| Cycle 13, Day 1 | Number | 6 | 5 | 21 |
| | Analyzed | participants | participants | participants |
| | | −5.8 (14.7) | −5.9 (8.8) | −0.6 (7.1) |
| Cycle 14, Day 1 | Number | 5 | 5 | 7 |
| | Analyzed | participants | participants | participants |
| | | −1.6 (17.1) | −5.0 (7.3) | −1.5 (7.7) |
| Cycle 15, Day 1 | Number | 6 | 4 | 7 |
| | Analyzed | participants | participants | participants |
| | | −4.8 (15.8) | −6.8 (7.0) | −3.2 (8.2) |
| Cycle 16, Day 1 | Number | 5 | 3 | 4 |
| | Analyzed | participants | participants | participants |
| | | −4.6 (16.1) | −6.9 (7.6) | 1.9 (7.9) |
| Cycle 17, Day 1 | Number | 5 | 3 | 5 |
| | Analyzed | participants | participants | participants |
| | | −4.1 (17.2) | −10.1 (5.5) | 0.0 (12.5) |
| Cycle 18, Day 1 | Number | 5 | 3 | 3 |
| | Analyzed | participants | participants | participants |
| | | −2.3 (17.6) | −11.5 (8.4) | −2.3 (7.8) |
| Cycle 19, Day 1 | Number | 5 | 3 | 4 |
| | Analyzed | participants | participants | participants |
| | | −7.7 (19.6) | −9.9 (7.8) | −6.6 (9.9) |
| Cycle 20, Day 1 | Number | 5 | 3 | 3 |
| | Analyzed | participants | participants | participants |
| | | −4.6 (22.0) | −9.0 (10.3) | −2.8 (5.6) |
| Cycle 21, Day 1 | Number | 5 | 3 | 4 |
| | Analyzed | participants | participants | participants |
| | | −3.1 (19.1) | −7.5 (10.7) | −4.0 (7.9) |
| Cycle 22, Day 1 | Number | 5 | 2 | 3 |
| | Analyzed | participants | participants | participants |
| | | −4.1 (18.9) | −14.5 (9.1) | −3.6 (5.0) |
| Cycle 23, Day 1 | Number | 5 | 2 | 4 |
| | Analyzed | participants | participants | participants |
| | | −4.1 (19.1) | −11.6 (9.3) | −6.2 (7.7) |
| Cycle 24, Day 1 | Number | 4 | 1 | 2 |
| | Analyzed | participants | participants | participants |
| | | −6.7 (21.0) | −18.7 (—) | −5.6 (5.7) |
| Cycle 25, Day 1 | Number | 2 | 2 | 3 |
| | Analyzed | participants | participants | participants |
| | | 2.2 (18.8) | −12.4 (8.2) | −8.4 (3.4) |
| Cycle 26, Day 1 | Number | 3 | 0 | 1 |
| | Analyzed | participants | participants | participants |
| | | 1.3 (14.3) | — | 0.2 (—) |
| Cycle 27, Day 1 | Number | 2 | 2 | 3 |
| | Analyzed | participants | participants | participants |
| | | −3.5 (25.3) | −13.2 (6.6) | −6.0 (9.6) |
| Cycle 28, Day 1 | Number | 3 | 0 | 1 |
| | Analyzed | participants | participants | participants |
| | | 4.4 (13.4) | — | 2.6 (—) |
| Cycle 29, Day 1 | Number | 2 | 2 | 2 |
| | Analyzed | participants | participants | participants |
| | | −2.8 (23.7) | −11.0 (7.3) | −3.7 (13.0) |
| Cycle 30, Day 1 | Number | 2 | 0 | 0 |
| | Analyzed | participants | participants | participants |
| | | 9.0 (7.5) | — | — |
| Cycle 31, Day 1 | Number | 2 | 2 | 1 |
| | Analyzed | participants | participants | participants |
| | | −4.8 (27.9) | −15.6 (4.3) | −12.9 (—) |
| Cycle 32, Day 1 | Number | 2 | 0 | 0 |
| | Analyzed | participants | participants | participants |
| | | 10.3 (6.9) | — | — |
| Cycle 33, Day 1 | Number | 2 | 2 | 1 |
| | Analyzed | participants | participants | participants |
| | | 0.5 (20.7) | −13.5 (9.0) | −12.9 (—) |
| Cycle 34, Day 1 | Number | 2 | 0 | 0 |
| | Analyzed | participants | participants | participants |
| | | 13.5 (3.6) | — | — |
| Cycle 35, Day 1 | Number | 2 | 2 | 1 |
| | Analyzed | participants | participants | participants |
| | | −2.5 (23.3) | −11.0 (12.4) | −12.9 (—) |
| Cycle 36, Day 1 | Number | 2 | 0 | 0 |
| | Analyzed | participants | participants | participants |
| | | 7.5 (6.4) | — | — |
| Cycle 37, Day 1 | Number | 2 | 2 | 0 |
| | Analyzed | participants | participants | participants |
| | | −5.3 (29.0) | −13.6 (8.1) | — |
| Cycle 38, Day 1 | Number | 2 | 0 | 0 |
| | Analyzed | participants | participants | participants |
| | | 9.1 (5.2) | — | — |
| Cycle 39, Day 1 | Number | 2 | 2 | 1 |
| | Analyzed | participants | participants | participants |
| | | −6.2 (25.9) | −14.3 (8.5) | −12.9 (—) |
| Cycle 40, Day 1 | Number | 1 participants | 0 | 0 |
| | Analyzed | | participants | participants |
| | | 2.7 (—) | — | — |
| Cycle 41, Day 1 | Number | 1 | 1 | 1 |
| | Analyzed | participants | participants | participants |
| | | −25.7 (—) | −19.3 (—) | −12.9 (—) |
| Cycle 42, Day 1 | Number | 1 | 0 | 0 |
| | Analyzed | participants | participants | participants |
| | | 4.6 (—) | — | — |
| Cycle 43, Day 1 | Number | 1 | 1 | 1 |
| | Analyzed | participants | participants | participants |
| | | −20.4 (—) | −19.9 (—) | −12.9 (—) |
| Cycle 44, Day 1 | Number | 1 | 0 | 0 |
| | Analyzed | participants | participants | participants |
| | | 4.3 (—) | — | — |
| Cycle 45, Day 1 | Number | 0 | 0 | 1 participants |
| | Analyzed | participants | participants | |
| | | — | — | −12.3 (—) |
| Cycle 46, Day 1 | Number | 1 | 0 | 0 |
| | Analyzed | participants | participants | participants |
| | | 4.6 (—) | — | — |
| Cycle 47, Day 1 | Number | 1 | 1 participants | 1 participants |
| | Analyzed | participants | | |
| | | −6.8 (—) | −16.8 (—) | −12.9 (—) |
| Cycle 48, Day 1 | Number | 1 | 0 | 0 |
| | Analyzed | participants | participants | participants |
| | | 2.4 (—) | — | — |
| Cycle 49, Day 1 | Number | 0 | 1 participants | 1 participants |
| | Analyzed | participants | | |
| | | — | −15.4 (—) | −14.3 (—) |
| Cycle 50, Day 1 | Number | 1 | 0 | 0 |
| | Analyzed | participants | participants | participants |
| | | 4.5 (—) | — | — |
| Cycle 51, Day 1 | Number | 1 | 0 | 1 participants |
| | Analyzed | participants | participants | |
| | | −12.0 (—) | — | −12.9 (—) |
| Cycle 52, Day 1 | Number | 1 | 0 | 0 |
| | Analyzed | participants | participants | participants |
| | | 6.0 (—) | — | — |
| Cycle 53, Day 1 | Number | 1 | 0 | 0 |
| | Analyzed | participants | participants | participants |
| | | −19.2 (—) | — | — |
| Cycle 54, Day 1 | Number | 1 | 0 | 0 |
| | Analyzed | participants | participants | participants |
| | | 4.9 (—) | — | — |
| Cycle 55, Day 1 | Number | 1 | 0 | 0 |
| | Analyzed | participants | participants | participants |
| | | −16.8 (—) | — | — |
| Cycle 56, Day 1 | Number | 1 | 0 | 0 |
| | Analyzed | participants | participants | participants |
| | | 1.8 (—) | — | — |
| Cycle 57, Day 1 | Number | 1 | 0 | 0 |
| | Analyzed | participants | participants | participants |
| | | −17.6 (—) | — | — |
| Cycle 58, Day 1 | Number | 1 | 0 | 0 |
| | Analyzed | participants | participants | participants |
| | | 5.4 (—) | — | — |
| Cycle 59, Day 1 | Number | 1 | 0 | 0 |
| | Analyzed | participants | participants | participants |
| | | −12.7 (—) | — | — |
| Cycle 60, Day 1 | Number | 1 | 0 | 0 |
| | Analyzed | participants | participants | participants |
| | | 4.2 (—) | — | — |
| Cycle 61, Day 1 | Number | 1 | 0 | 0 |
| | Analyzed | participants | participants | participants |
| | | −19.3 (—) | — | — |
| Cycle 62, Day 1 | Number | 1 | 0 | 0 |
| | Analyzed | participants | participants | participants |
| | | 4.4 (—) | — | — |
| Cycle 63, Day 1 | Number | 1 | 0 | 0 |
| | Analyzed | participants | participants | participants |
| | | −18.0 (—) | — | — |
| Cycle 67, Day 1 | Number | 1 | 0 | 0 |
| | Analyzed | participants | participants | participants |
| | | −21.9 (—) | — | — |
| EOT | Number | 10 | 8 | 10 |
| | Analyzed | participants | participants | participants |
| | | −4.3 (11.7) | −2.4 (10.9) | 2.5 (11.9) |

Change From Baseline in Percent Predicted Forced Expiratory Volume in 1 Second (FEV1) at Each Specified Time Points. FEV1 was the volume of air exhaled from the lungs in the first second of a forced expiration as measured by spirometer. Baseline value was defined as valid and last non-missing value obtained within 28 days prior to participant receiving first study medication. EOT visit was performed within 3 days after the participant's last dose of study drug. Timeframe: Baseline, Day 1 of Cycles 2, 3, 4, 5, 6, 7, 8, 9, 10, 11, 12, 13, 14, 15, 16, 17, 18, 19, 20, 21, 22, 23, 24, 25, 26, 27, 29, 30, 31, 32, 33, 34, 35, 36, 37, 38, 39, 40, 41, 43, 47, 48, 49, 51, 52, 53, 54, 55, 56, 57, 58, 60, 61, 62, 63, 67, EOT (i.e., anytime up to 64.2 months). Analysis was performed on mITT population. Here, ‘number analyzed’=participants with available data for each specified category. Here, ‘O’ in the number analyzed field signifies that none of the participants were available for the analysis at the specified time points. The data is shown in the table below.

TABLE 22
Change From Baseline in Percent Predicted Forced Expiratory
Volume in 1 Second (FEV1) at Each Specified Time Points

| | | Cohort 1: | Cohort 2: | Cohort 3: |
|---|---|---|---|---|
| | | Belumosudil | Belumosudil | Belumosudil |
| | | 200 mg QD | 200 mg BID | 400 mg QD |
| Overall Number of | | 17 | 16 | 21 |
| Participants Analyzed | | | | |
| Change From | | | | |
| Baseline | | | | |
| in Percent | | | | |
| Predicted | | | | |
| Forced | | | | |
| Expiratory | | | | |
| Volume in 1 | | | | |
| Second (FEV1) | | | | |
| at Each | | | | |
| Specified | | | | |
| Time Points | | | | |
| Mean | | | | |
| (Standard | | | | |
| Deviation) | | | | |
| Unit of | | | | |
| measure: | | | | |
| percent | | | | |
| predicted | | | | |
| FEV1 | | | | |
| Cycle 2, Day 1 | Number | 0 | 0 | 4 |
| | Analyzed | participants | participants | participants |
| | | — | — | −4.8 (6.7) |
| Cycle 3, Day 1 | Number | 16 | 14 | 12 |
| | Analyzed | participants | participants | participants |
| | | 1.1 (4.8) | −1.8 (7.1) | −1.5 (6.2) |
| Cycle 4, Day 1 | Number | 0 | 0 | 21 |
| | Analyzed | participants | participants | participants |
| | | — | — | −0.2 (8.4) |
| Cycle 5, Day 1 | Number | 13 | 11 | 12 |
| | Analyzed | participants | participants | participants |
| | | −3.3 (8.0) | −5.0 (6.5) | 0.5 (5.9) |
| Cycle 6, Day 1 | Number | 0 | 0 | 8 |
| | Analyzed | participants | participants | participants |
| | | — | — | −0.9 (6.4) |
| Cycle 7, Day 1 | Number | 11 | 9 | 13 |
| | Analyzed | participants | participants | participants |
| | | −3.5 (4.6) | −2.6 (9.4) | −0.9 (11.8) |
| Cycle 8, Day 1 | Number | 0 | 1 | 7 |
| | Analyzed | participants | participants | participants |
| | | — | 1.0 (—) | −1.1 (8.0) |
| Cycle 9, Day 1 | Number | 9 | 8 | 10 |
| | Analyzed | participants | participants | participants |
| | | −1.7 (6.8) | −5.9 (11.7) | −3.0 (11.8) |
| Cycle 10, Day 1 | Number | 0 | 2 | 6 |
| | Analyzed | participants | participants | participants |
| | | — | −6.0 (8.5) | 2.7 (3.9) |
| Cycle 11, Day 1 | Number | 7 | 6 | 8 |
| | Analyzed | participants | participants | participants |
| | | −4.3 (5.2) | −4.0 (8.5) | −4.4 (15.8) |
| Cycle 12, Day 1 | Number | 1 | 1 | 7 |
| | Analyzed | participants | participants | participants |
| | | −2.0 (—) | −1.0 (—) | 0.3 (3.6) |
| Cycle 13, Day 1 | Number | 7 | 4 | 8 |
| | Analyzed | participants | participants | participants |
| | | −5.9 (9.3) | −2.5 (14.7) | −3.5 (13.2) |
| Cycle 14, Day 1 | Number | 1 | 1 | 7 |
| | Analyzed | participants | participants | participants |
| | | 2.0 (—) | 0.0 (—) | −5.1 (9.1) |
| Cycle 15, Day 1 | Number | 6 | 3 | 8 |
| | Analyzed | participants | participants | participants |
| | | −6.7 (11.7) | −2.3 (5.1) | −6.1 (12.6) |
| Cycle 16, Day 1 | Number | 1 | 42 | 4 |
| | Analyzed | participants | participants | participants |
| | | 2.0 (—) | −8.0 (15.6) | −4.5 (7.3) |
| Cycle 17, Day 1 | Number | 6 | 3 | 47 |
| | Analyzed | participants | participants | participants |
| | | −5.3 (9.1) | −6.0 (7.2) | −7.9 (15.5) |
| Cycle 18, Day 1 | Number | 0 | 2 | 3 |
| | Analyzed | participants | participants | participants |
| | | — | −7.5 (10.6) | 4.0 (6.0) |
| Cycle 19, Day 1 | Number | 6 | 43 | 4 |
| | Analyzed | participants | participants | participants |
| | | −5.2 (9.9) | −6.3 (5.5) | 0.5 (8.5) |
| Cycle 20, Day 1 | Number | 0 | 42 | 3 |
| | Analyzed | participants | participants | participants |
| | | — | −8.0 (7.1) | 4.0 (10.4) |
| Cycle 21, Day 1 | Number | 5 | 1 | 21 |
| | Analyzed | participants | participants | participants |
| | | −7.4 (10.6) | 0.0 (—) | −10.0 (19.8) |
| Cycle 22, Day 1 | Number | 0 | 2 | 2 |
| | Analyzed | participants | participants | participants |
| | | — | −11.0 (1.4) | −6.0 (17.0) |
| Cycle 23, Day 1 | Number | 3 | 2 | 4 |
| | Analyzed | participants | participants | participants |
| | | −4.3 (14.6) | −12.5 (4.9) | −4.8 (11.2) |
| Cycle 24, Day 1 | Number | 1 | 1 | 1 |
| | Analyzed | participants | participants | participants |
| | | 2.0 (—) | −18.0 (—) | −19.0 (—) |
| Cycle 25, Day 1 | Number | 2 | 2 | 3 |
| | Analyzed | participants | participants | participants |
| | | −11.0 (15.6) | −12.5 (3.5) | −5.0 (11.4) |
| Cycle 26, Day 1 | Number | 1 | 0 | 0 |
| | Analyzed | participants | participants | participants |
| | | 0.0 (—) | — | — |
| Cycle 27, Day 1 | Number | 2 | 2 | 2 |
| | Analyzed | participants | participants | participants |
| | | −6.5 (13.4) | −17.0 (9.9) | −10.0 (17.0) |
| Cycle 29, Day 1 | Number | 2 | 2 | 2 |
| | Analyzed | participants | participants | participants |
| | | −0.5 (9.2) | 17.0 (11.3) | 0.0 (1.4) |
| Cycle 30, Day 1 | Number | 1 | 0 | 0 |
| | Analyzed | participants | participants | participants |
| | | 3.0 (—) | — | — |
| Cycle 31, Day 1 | Number | 2 | 2 | 1 |
| | Analyzed | participants | participants | participants |
| | | −3.5 (12.0) | −19.5 (10.6) | −4.0 (—) |
| Cycle 32, Day 1 | Number | 1 | 0 | 0 |
| | Analyzed | participants | participants | participants |
| | | 1.0 (—) | — | — |
| Cycle 33, Day 1 | Number | 2 | 2 | 0 |
| | Analyzed | participants | participants | participants |
| | | 5.0 (5.7) | −1.0 (33.9) | — |
| Cycle 34, Day 1 | Number | 1 | 0 | 0 |
| | Analyzed | participants | participants | participants |
| | | 2.0 (—) | — | — |
| Cycle 35, Day 1 | Number | 17 | 2 | 1 |
| | Analyzed | participants | participants | participants |
| | | 5.0 (0.0) | −2.5 (30.4) | −4.0 (—) |
| Cycle 36, Day 1 | Number | 1 | 0 | 1 |
| | Analyzed | participants | participants | participants |
| | | 3.0 (—) | — | 5.0 (—) |
| Cycle 37, Day 1 | Number | 1 | 1 | 0 |
| | Analyzed | participants | participants | participants |
| | | −1.0 (—) | −22.0 (—) | — |
| Cycle 38, Day 1 | Number | 1 | 0 | 0 |
| | Analyzed | participants | participants | participants |
| | | 1.0 (—) | — | — |
| Cycle 39, Day 1 | Number | 2 | 2 | 1 |
| | Analyzed | participants | participants | participants |
| | | 10.0 (2.8) | −23.0 (11.3) | −5.0 (—) |
| Cycle 40, Day 1 | Number | 1 | 0 | 0 |
| | Analyzed | participants | participants | participants |
| | | −1.0 (—) | — | — |
| Cycle 41, Day 1 | Number | 1 | 0 | 0 |
| | Analyzed | participants | participants | participants |
| | | 15.0 (—) | — | — |
| Cycle 43, Day 1 | Number | 1 | 1 | 1 |
| | Analyzed | participants | participants | participants |
| | | 16.0 (—) | −21.0 (—) | −7.0 (—) |
| Cycle 47, Day 1 | Number | 1 | 0 | 1 |
| | Analyzed | participants | participants | participants |
| | | 5.0 (—) | — | −5.0 (—) |
| Cycle 48, Day 1 | Number | 1 | 0 | 0 |
| | Analyzed | participants | participants | participants |
| | | 3.0 (—) | — | — |
| Cycle 49, Day 1 | Number | 1 | 0 | 1 |
| | Analyzed | participants | participants | participants |
| | | 8.0 (—) | — | −2.0 (—) |
| Cycle 51, Day 1 | Number | 1 | 0 | 0 |
| | Analyzed | participants | participants | participants |
| | | 8.0 (—) | — | — |
| Cycle 52, Day 1 | Number | 1 | 0 | 0 |
| | Analyzed | participants | participants | participants |
| | | 0.0 (—) | — | — |
| Cycle 53, Day 1 | Number | 1 | 0 | 0 |
| | Analyzed | participants | participants | participants |
| | | 9.0 (—) | — | — |
| Cycle 54, Day 1 | Number | 1 | 0 | 0 |
| | Analyzed | participants | participants | participants |
| | | 4.0 (—) | — | — |
| Cycle 55, Day 1 | Number | 1 | 0 | 0 |
| | Analyzed | participants | participants | participants |
| | | 13.0 (—) | — | — |
| Cycle 56, Day 1 | Number | 1 | 0 | 0 |
| | Analyzed | participants | participants | participants |
| | | 4.0 (—) | — | — |
| Cycle 57, Day 1 | Number | 1 | 0 | 0 |
| | Analyzed | participants | participants | participants |
| | | 13.0 (—) | — | — |
| Cycle 58, Day 1 | Number | 1 | 0 | 0 |
| | Analyzed | participants | participants | participants |
| | | 5.0 (—) | — | — |
| Cycle 60, Day 1 | Number | 1 | 0 | 0 |
| | Analyzed | participants | participants | participants |
| | | 4.0 (—) | — | — |
| Cycle 61, Day 1 | Number | 1 | 0 | 0 |
| | Analyzed | participants | participants | participants |
| | | 10.0 (—) | — | — |
| Cycle 62, Day 1 | Number | 1 | 0 | 0 |
| | Analyzed | participants | participants | participants |
| | | 2.0 (—) | — | — |
| Cycle 63, Day 1 | Number | 1 | 0 | 0 |
| | Analyzed | participants | participants | participants |
| | | 13.0 (—) | — | — |
| Cycle 67, Day 1 | Number | 1 | 0 | 0 |
| | Analyzed | participants | participants | participants |
| | | 14.0 (—) | — | — |
| EOT | Number | 12 | 10 | 9 |
| | Analyzed | participants | participants | participants |
| | | −1.4 (9.0) | −10.1 (15.7) | −7.4 (14.9) |

Change From Baseline in Percent Predicted Forced Vital Capacity (FVC) at Each Specified Time Points. FVC was the total amount of air (in liters) exhaled from the lungs during the lung function test measured by spirometer which assessed the change in lung function related to the disease status. Baseline value was defined as valid and last non-missing value obtained within 28 days prior to participant receiving first study medication. EOT visit was performed within 3 days after the participant's last dose of study drug. Timeframe: Baseline, Day 1 of Cycles 2, 3, 4, 5, 6, 7, 8, 9, 10, 11, 12, 13, 14, 15, 16, 17, 18, 19, 20, 21, 22, 23, 24, 25, 26, 27, 29, 30, 31, 32, 33, 34, 35, 36, 37, 38, 39, 40, 41, 43, 47, 48, 49, 51, 52, 53, 54, 55, 56, 57, 58, 60, 61, 62, 63, 67, EOT (i.e., anytime up to 64.2 months). Analysis was performed on mITT population. Here, ‘number analyzed’=participants with available data for each specified category. Here, ‘0’ in the number analyzed field signifies that none of the participants were available for the analysis at the specified time points. The data is shown in the table below.

TABLE 23
Change From Baseline in Percent Predicted Forced Vital Capacity (FVC)
at Each Specified Time Points

| | | Cohort 1: | Cohort 2: | Cohort 3: |
|---|---|---|---|---|
| | | Belumosudil | Belumosudil | Belumosudil |
| | | 200 mg QD | 200 mg BID | 400 mg QD |
| Overall Number of Participants Analyzed | | 17 | 16 | 21 |
| Change From Baseline | | | | |
| in Percent Predicted | | | | |
| Forced Vital Capacity | | | | |
| (FVC) at Each | | | | |
| Specified Time Points | | | | |
| Mean (Standard | | | | |
| Deviation) | | | | |
| Unit of measure: | | | | |
| percent predicted FVC | | | | |
| Cycle 2, Day 1 | Number Analyzed | 0 participants | 0 participants | 21 participants |
| | | — | — | −5.5 (9.9) |
| Cycle 3, Day 1 | Number Analyzed | 16 participants | 14 participants | 12 participants |
| | | 0.5 (3.8) | −2.4 (6.0) | −3.0 (6.7) |
| Cycle 4, Day 1 | Number Analyzed | 0 participants | 0 participants | 6 participants |
| | | — | — | −0.2 (8.6) |
| Cycle 5, Day 1 | Number Analyzed | 13 participants | 11 participants | 12 participants |
| | | −0.5 (7.1) | −4.3 (5.2) | 1.6 (6.3) |
| Cycle 6, Day 1 | Number Analyzed | 0 participants | 0 participants | 8 participants |
| | | — | — | 0.8 (9.5) |
| Cycle 7, Day 1 | Number Analyzed | 11 participants | 9 participants | 13 participants |
| | | −2.9 (5.2) | −1.3 (6.4) | 2.3 (9.7) |
| Cycle 8, Day 1 | Number Analyzed | 0 participants | 1 participants | 7 participants |
| | | — | 2.0 (—) | 0.4 (8.9) |
| Cycle 9, Day 1 | Number Analyzed | 9 participants | 8 participants | 10 participants |
| | | 0.4 (5.1) | −2.9 (5.5) | −0.8 (9.5) |
| Cycle 10, Day 1 | Number Analyzed | 0 participants | 2 participants | 6 participants |
| | | — | −5.0 (8.5) | 4.7 (4.9) |
| Cycle 11, Day 1 | Number Analyzed | 7 participants | 6 participants | 8 participants |
| | | −1.1 (7.3) | −2.7 (5.6) | −4.4 (12.0) |
| Cycle 12, Day 1 | Number Analyzed | 1 participants | 1 participants | 7 participants |
| | | 2.0 (—) | 1.0 (—) | 0.7 (5.8) |
| Cycle 13, Day 1 | Number Analyzed | 7 participants | 4 participants | 8 participants |
| | | −2.4 (7.9) | −3.3 (15.4) | −4.9 (9.8) |
| Cycle 14, Day 1 | Number Analyzed | 1 participants | 1 participants | 7 participants |
| | | 11.0 (—) | 2.0 (— ) | −2.7 (7.1) |
| Cycle 15, Day 1 | Number Analyzed | 6 participants | 3 participants | 8 participants |
| | | −3.7 (10.4) | −3.3 (5.5) | −4.8 (11.5) |
| Cycle 16, Day 1 | Number Analyzed | 1 participants | 2 participants | 4 participants |
| | | 16.0 (—) | −6.0 (19.8) | −6.5 (6.5) |
| Cycle 17, Day 1 | Number Analyzed | 6 participants | 3 participants | 7 participants |
| | | −3.2 (6.3) | −3.0 (11.8) | −5.6 (12.8) |
| Cycle 18, Day 1 | Number Analyzed | 0 participants | 2 participants | 3 participants |
| | | — | −7.5 (9.2) | 4.0 (4.0) |
| Cycle 19, Day 1 | Number Analyzed | 6 participants | 3 participants | 4 participants |
| | | −2.3 (6.8) | −5.3 (4.7) | 0.8 (5.1) |
| Cycle 20, Day 1 | Number Analyzed | 0 participants | 2 participants | 3 participants |
| | | — | −10.5 (2.1) | −2.0 (5.6) |
| Cycle 21, Day 1 | Number Analyzed | 5 participants | 1 participants | 5 participants |
| | | −3.2 (8.1) | −3.0 (—) | −6.6 (17.0) |
| Cycle 22, Day 1 | Number Analyzed | 0 participants | 2 participants | 2 participants |
| | | — | −11.0 (0.0) | −6.0 (8.5) |
| Cycle 23, Day 1 | Number Analyzed | 3 participants | 2 participants | 4 participants |
| | | 0.0 (12.3) | −11.0 (2.8) | −1.8 (10.6) |
| Cycle 24, Day 1 | Number Analyzed | 1 participants | 1 participants | 1 participants |
| | | 4.0 (—) | −13.0 (—) | −15.0 (—) |
| Cycle 25, Day 1 | Number Analyzed | 2 participants | 2 participants | 3 participants |
| | | −7.0 (11.3) | −10.5 (3.5) | −1.3 (11.7) |
| Cycle 26, Day 1 | Number Analyzed | 1 participants | 0 participants | 0 participants |
| | | 4.0 (—) | — | — |
| Cycle 27, Day 1 | Number Analyzed | 2 participants | 2 participants | 2 participants |
| | | −2.0 (4.2) | −18.0 (12.7) | −4.0 (21.2) |
| Cycle 29, Day 1 | Number Analyzed | 2 participants | 1 participants | 2 participants |
| | | 2.5 (4.9) | −11.0 (—) | 6.0 (1.4) |
| Cycle 30, Day 1 | Number Analyzed | 1 participants | 0 participants | 0 participants |
| | | 4.0 (—) | — | — |
| Cycle 31, Day 1 | Number Analyzed | 2 participants | 2 participants | 1 participants |
| | | 1.5 (3.5) | −15.5 (7.8) | −4.0 (—) |
| Cycle 32, Day 1 | Number Analyzed | 1 participants | 0 participants | 0 participants |
| | | 2.0 (—) | — | — |
| Cycle 33, Day 1 | Number Analyzed | 2 participants | 2 participants | 0 participants |
| | | 11.0 (0.0) | −17.5 (7.8) | — |
| Cycle 34, Day 1 | Number Analyzed | 1 participants | 0 participants | 0 participants |
| | | 6.0 (—) | — | — |
| Cycle 35, Day 1 | Number Analyzed | 2 participants | 2 participants | 1 participants |
| | | 11.0 (5.7) | −15.0 (11.3) | −3.0 (—) |
| Cycle 36, Day 1 | Number Analyzed | 1 participants | 0 participants | 1 participants |
| | | 5.0 (—) | — | 2.0 (—) |
| Cycle 37, Day 1 | Number Analyzed | 1 participants | 1 participants | 0 participants |
| | | 7.0 (—) | −24.0 (—) | — |
| Cycle 38, Day 1 | Number Analyzed | 1 participants | 0 participants | 0 participants |
| | | 2.0 (—) | — | — |
| Cycle 39, Day 1 | Number Analyzed | 2 participants | 2 participants | 1 participants |
| | | 12.5 (0.7) | −20.5 (12.0) | −1.0 (—) |
| Cycle 40, Day 1 | Number Analyzed | 1 participants | 0 participants | 0 participants |
| | | 3.0 (—) | — | — |
| Cycle 41, Day 1 | Number Analyzed | 1 participants | 0 participants | 0 participants |
| | | 15.0 (—) | — | — |
| Cycle 43, Day 1 | Number Analyzed | 1 participants | 1 participants | 1 participants |
| | | 17.0 (—) | −18.0 (—) | −4.0 (—) |
| Cycle 47, Day 1 | Number Analyzed | 1 participants | 0 participants | 1 participants |
| | | 5.0 (—) | — | −2.0 (—) |
| Cycle 48, Day 1 | Number Analyzed | 1 participants | 0 participants | 0 participants |
| | | 5.0 (—) | — | — |
| Cycle 49, Day 1 | Number Analyzed | 1 participants | 0 participants | 1 participants |
| | | 6.0 (—) | — | 2.0 (—) |
| Cycle 51, Day 1 | Number Analyzed | 1 participants | 0 participants | 0 participants |
| | | 9.0 (—) | — | — |
| Cycle 52, Day 1 | Number Analyzed | 1 participants | 0 participants | 0 participants |
| | | 3.0 (—) | — | — |
| Cycle 53, Day 1 | Number Analyzed | 1 participants | 0 participants | 0 participants |
| | | 8.0 (—) | — | — |
| Cycle 54, Day 1 | Number Analyzed | 1 participants | 0 participants | 0 participants |
| | | 5.0 (—) | — | — |
| Cycle 55, Day 1 | Number Analyzed | 1 participants | 0 participants | 0 participants |
| | | 13.0 (—) | — | — |
| Cycle 56, Day 1 | Number Analyzed | 1 participants | 0 participants | 0 participants |
| | | 7.0 (—) | — | — |
| Cycle 57, Day 1 | Number Analyzed | 1 participants | 0 participants | 0 participants |
| | | 14.0 (—) | — | — |
| Cycle 58, Day 1 | Number Analyzed | 1 participants | 0 participants | 0 participants |
| | | 7.0 (—) | — | — |
| Cycle 60, Day 1 | Number Analyzed | 1 participants | 0 participants | 0 participants |
| | | 8.0 (—) | — | — |
| Cycle 61, Day 1 | Number Analyzed | 1 participants | 0 participants | 0 participants |
| | | 12.0 (—) | — | — |
| Cycle 62, Day 1 | Number Analyzed | 1 participants | 0 participants | 0 participants |
| | | 5.0 (—) | — | — |
| Cycle 63, Day 1 | Number Analyzed | 1 participants | 0 participants | 0 participants |
| | | 12.0 (—) | — | — |
| Cycle 67, Day 1 | Number Analyzed | 1 participants | 0 participants | 0 participants |
| | | 14.0 (—) | — | — |
| EOT | Number Analyzed | 12 participants | 10 participants | 9 participants |
| | | −2.5 (10.3) | −8.4 (9.2) | −7.8 (14.7) |

Change From Baseline in Percent Predicted Hemoglobin (HGB) Corrected Diffusing Capacity of Lung for Carbon Monoxide (DLco) at Each Specified Time Points. DLco is a measurement of the ability of the lungs to transfer gases from the air to the blood. Change from baseline in diffusing capacity of the lung for carbon monoxide (percent predicted hemoglobin level corrected) was reported for this measure. Baseline value was defined as valid and last non-missing value obtained within 28 days prior to participant receiving first study medication. EOT visit was performed within 3 days after the participant's last dose of study drug. Timeframe: Baseline, Day 1 of Cycles 2, 3, 4, 5, 6, 7, 8, 9, 10, 11, 12, 13, 14, 15, 16, 17, 18, 19, 20, 21, 22, 23, 24, 25, 26, 27, 29, 30, 31, 32, 33, 34, 35, 36, 37, 38, 39, 40, 41, 43, 47, 48, 49, 51, 52, 53, 54, 55, 56, 57, 58, 60, 61, 62, 63, 67, EOT (i.e., anytime up to 64.2 months). Analysis was performed on mITT population. Here, ‘number analyzed’=participants with available data for each specified category. Here, ‘O’ in the number analyzed field signifies that none of the participants were available for the analysis at the specified time points. The data is shown in the table below.

TABLE 24
Change From Baseline in Percent Predicted Hemoglobin (HGB) Corrected
Diffusing Capacity of Lung for Carbon Monoxide (DLco) at Each Specified Time Points

| | | Cohort 1: | Cohort 2: | Cohort 3: |
|---|---|---|---|---|
| | | Belumosudil | Belumosudil | Belumosudil |
| | | 200 mg QD | 200 mg BID | 400 mg QD |
| Overall Number of Participants Analyzed | | 17 | 16 | 21 |
| Change From Baseline | | | | |
| in Percent Predicted | | | | |
| Hemoglobin (HGB) | | | | |
| Corrected Diffusing | | | | |
| Capacity of Lung for | | | | |
| Carbon Monoxide | | | | |
| (DLco) at Each | | | | |
| Specified Time Points | | | | |
| Mean (Standard | | | | |
| Deviation) | | | | |
| Unit of measure: | | | | |
| Percent Predicted | | | | |
| HGB Corrected DLco | | | | |
| Cycle 2, Day 1 | Number Analyzed | 0 participants | 0 participants | 4 participants |
| | | — | — | −5.243 (9.830) |
| Cycle 3, Day 1 | Number Analyzed | 14 participants | 14 participants | 9 participants |
| | | 4.545 (22.991) | −0.638 (7.059) | −3.928 (9.492) |
| Cycle 4, Day 1 | Number Analyzed | 0 participants | 0 participants | 5 participants |
| | | — | — | 0.278 (9.683) |
| Cycle 5, Day 1 | Number Analyzed | 12 participants | 12 participants | 9 participants |
| | | 4.635 (23.989) | −4.462 (23.621) | −4.111 (9.138) |
| Cycle 6, Day 1 | Number Analyzed | 0 participants | 0 participants | 7 participants |
| | | — | — | −3.143 (10.385) |
| Cycle 7, Day 1 | Number Analyzed | 10 participants | 8 participants | 7 participants |
| | | 1.521 (19.836) | 6.184 (9.436) | −4.275 (11.464) |
| Cycle 8, Day 1 | Number Analyzed | 0 participants | 1 participants | 6 participants |
| | | — | 3.554 (—) | −3.787 (11.983) |
| Cycle 9, Day 1 | Number Analyzed | 9 participants | 8 participants | 8 participants |
| | | −4.514 (7.401) | 2.570 (8.902) | −2.540 (11.897) |
| Cycle 10, Day 1 | Number Analyzed | 0 participants | 2 participants | 5 participants |
| | | — | 4.343 (3.144) | 1.328 (8.887) |
| Cycle 11, Day 1 | Number Analyzed | 6 participants | 6 participants | 7 participants |
| | | −0.625 (7.262) | 6.582 (10.695) | −2.720 (10.920) |
| Cycle 12, Day 1 | Number Analyzed | 1 participants | 1 participants | 6 participants |
| | | 0.531 (—) | 4.076 (—) | −1.378 (6.364) |
| Cycle 13, Day 1 | Number Analyzed | 7 participants | 4 participants | 7 participants |
| | | −5.548 (12.175) | 1.882 (7.522) | 2.091 (13.290) |
| Cycle 14, Day 1 | Number Analyzed | 1 participants | 1 participants | 6 participants |
| | | 12.916 (—) | 0.358 (—) | 0.309 (11.651) |
| Cycle 15, Day 1 | Number Analyzed | 6 participants | 3 participants | 7 participants |
| | | 0.228 (15.731) | 1.125 (5.927) | −5.309 (11.514) |
| Cycle 16, Day 1 | Number Analyzed | 1 participants | 2 participants | 4 participants |
| | | 48.465 (—) | −6.618 (15.443) | −6.840 (15.053) |
| Cycle 17, Day 1 | Number Analyzed | 6 participants | 3 participants | 6 participants |
| | | −1.018 (9.226) | 8.458 (6.229) | −10.865 (13.346) |
| Cycle 18, Day 1 | Number Analyzed | 0 participants | 2 participants | 3 participants |
| | | — | 15.543 (11.753) | −3.174 (3.106) |
| Cycle 19, Day 1 | Number Analyzed | 6 participants | 3 participants | 3 participants |
| | | −2.293 (5.763) | 1.824 (8.539) | −2.020 (2.436) |
| Cycle 20, Day 1 | Number Analyzed | 0 participants | 1 participants | 3 participants |
| | | — | −14.692 (—) | −13.517 (5.936) |
| Cycle 21, Day 1 | Number Analyzed | 5 participants | 1 participants | 4 participants |
| | | −2.778 (8.187) | −10.661 (—) | −12.354 (12.800) |
| Cycle 22, Day 1 | Number Analyzed | 0 participants | 2 participants | 2 participants |
| | | — | 3.347 (3.560) | −17.222 (16.947) |
| Cycle 23, Day 1 | Number Analyzed | 2 participants | 2 participants | 3 participants |
| | | 0.354 (1.844) | −15.184 (20.181) | −2.219 (10.614) |
| Cycle 24, Day 1 | Number Analyzed | 1 participants | 1 participants | 1 participants |
| | | 6.945 (—) | −18.095 (—) | −11.430 (—) |
| Cycle 25, Day 1 | Number Analyzed | 2 participants | 2 participants | 1 participants |
| | | −4.461 (19.865) | 6.289 (2.469) | −13.392 (—) |
| Cycle 26, Day 1 | Number Analyzed | 1 participants | 0 participants | 0 participants |
| | | 4.715 (—) | — | — |
| Cycle 27, Day 1 | Number Analyzed | 2 participants | 2 participants | 1 participants |
| | | −8.047 (24.788) | −9.914 (15.387) | −11.154 (—) |
| Cycle 29, Day 1 | Number Analyzed | 2 participants | 2 participants | 1 participants |
| | | −6.701 (1.671) | −18.814 (24.960) | 8.613 (—) |
| Cycle 31, Day 1 | Number Analyzed | 2 participants | 2 participants | 1 participants |
| | | 2.343 (20.988) | −0.800 (17.528) | 10.833 (—) |
| Cycle 32, Day 1 | Number Analyzed | 1 participants | 0 participants | 0 participants |
| | | 5.586 (—) | — | — |
| Cycle 33, Day 1 | Number Analyzed | 2 participants | 2 participants | 0 participants |
| | | 9.901 (9.954) | −5.962 (10.550) | — |
| Cycle 34, Day 1 | Number Analyzed | 1 participants | 0 participants | 0 participants |
| | | 4.863 (—) | — | — |
| Cycle 35, Day 1 | Number Analyzed | 2 participants | 2 participants | 1 participants |
| | | 13.867 (1.309) | −0.426 (6.677) | 5.088 (—) |
| Cycle 36, Day 1 | Number Analyzed | 1 participants | 0 participants | 1 participants |
| | | 4.166 (—) | — | 10.372 (—) |
| Cycle 37, Day 1 | Number Analyzed | 1 participants | 1 participants | 0 participants |
| | | 9.306 (—) | −19.228 (—) | — |
| Cycle 39, Day 1 | Number Analyzed | 2 participants | 2 participants | 1 participants |
| | | 14.754 (0.649) | −6.813 (12.810) | 5.902 (—) |
| Cycle 40, Day 1 | Number Analyzed | 1 participants | 0 participants | 0 participants |
| | | 5.586 (—) | — | — |
| Cycle 41, Day 1 | Number Analyzed | 1 participants | 0 participants | 0 participants |
| | | 22.884 (—) | — | — |
| Cycle 43, Day 1 | Number Analyzed | 1 participants | 1 participants | 1 participants |
| | | 15.213 (—) | 8.775 (—) | 16.351 (—) |
| Cycle 47, Day 1 | Number Analyzed | 1 participants | 0 participants | 0 participants |
| | | 9.660 (—) | — | — |
| Cycle 49, Day 1 | Number Analyzed | 1 participants | 0 participants | 0 participants |
| | | −3.599 (—) | — | — |
| Cycle 51, Day 1 | Number Analyzed | 1 participants | 0 participants | 0 participants |
| | | 11.734 (—) | — | — |
| Cycle 53, Day 1 | Number Analyzed | 1 participants | 0 participants | 0 participants |
| | | 10.292 (—) | — | — |
| Cycle 55, Day 1 | Number Analyzed | 1 participants | 0 participants | 0 participants |
| | | 16.550 (—) | — | — |
| Cycle 61, Day 1 | Number Analyzed | 1 participants | 0 participants | 0 participants |
| | | 17.682 (—) | — | — |
| Cycle 62, Day 1 | Number Analyzed | 1 participants | 0 participants | 0 participants |
| | | 4.170 (—) | — | — |
| Cycle 63, Day 1 | Number Analyzed | 1 participants | 0 participants | 0 participants |
| | | 26.215 (—) | — | — |
| Cycle 67, Day 1 | Number Analyzed | 1 participants | 0 participants | 0 participants |
| | | 41.384 (—) | — | — |
| EOT | Number Analyzed | 9 participants | 6 participants | 7 participants |
| | | −1.962 (11.426) | 0.985 (5.919) | −10.371 (14.208) |

Change From Baseline in Percent Predicted Total Lung Capacity (TLC) at Each Specified Time Points. TLC is the volume of air in the lungs upon the maximum effort of inspiration. Baseline value was defined as valid and last non-missing value obtained within 28 days prior to participant receiving first study medication. EOT visit was performed within 3 days after the participant's last dose of study drug. Timeframe: Baseline, Day 1 of Cycles 2, 3, 4, 5, 6, 7, 8, 9, 10, 11, 12, 13, 14, 15, 16, 17, 18, 19, 20, 21, 22, 23, 24, 25, 26, 27, 29, 30, 31, 32, 33, 34, 35, 36, 37, 38, 39, 40, 41, 43, 47, 48, 49, 51, 52, 53, 54, 55, 56, 57, 58, 60, 61, 62, 63, 67, EOT (i.e., anytime up to 64.2 months). Analysis was performed on mITT population. Here, ‘number analyzed’=participants with available data for each specified category. Here, ‘0’ in the number analyzed field signifies that none of the participants were available for the analysis at the specified time points. The data is shown in the table below.

TABLE 25
Change From Baseline in Percent Predicted Total Lung Capacity (TLC) at Each
Specified Time Points

| | | Cohort 1: | Cohort 2: | Cohort 3: |
|---|---|---|---|---|
| | | Belumosudil | Belumosudil | Belumosudil |
| | | 200 mg QD | 200 mg BID | 400 mg QD |
| Overall Number of Participants Analyzed | | 17 | 16 | 21 |
| Change From Baseline | | | | |
| in Percent Predicted | | | | |
| Total Lung Capacity | | | | |
| (TLC) at Each | | | | |
| Specified Time Points | | | | |
| Mean (Standard Deviation) | | | | |
| Unit of measure: | | | | |
| Percent Predicted TLC | | | | |
| Cycle 2, Day 1 | Number Analyzed | 0 participants | 0 participants | 4 participants |
| | | — | — | −9.0 (13.4) |
| Cycle 3, Day 1 | Number Analyzed | 14 participants | 12 participants | 12 participants |
| | | 1.6 (11.9) | 1.9 (8.5) | −0.6 (14.8) |
| Cycle 4, Day 1 | Number Analyzed | 0 participants | 0 participants | 6 participants |
| | | — | — | 3.3 (14.0) |
| Cycle 5, Day 1 | Number Analyzed | 13 participants | 9 participants | 10 participants |
| | | −2.6 (16.8) | −7.2 (10.5) | −1.7 (11.2) |
| Cycle 6, Day 1 | Number Analyzed | 0 participants | 0 participants | 7 participants |
| | | — | — | −4.4 (11.6) |
| Cycle 7, Day 1 | Number Analyzed | 11 participants | 7 participants | 12 participants |
| | | −3.7 (6.6) | −4.4 (6.3) | −2.6 (13.4) |
| Cycle 8, Day 1 | Number Analyzed | 0 participants | 1 participants | 6 participants |
| | | — | −5.0 (—) | −0.2 (12.9) |
| Cycle 9, Day 1 | Number Analyzed | 9 participants | 8 participants | 10 participants |
| | | −2.9 (6.1) | −6.0 (8.5) | −0.9 (15.8) |
| Cycle 10, Day 1 | Number Analyzed | 0 participants | 2 participants | 5 participants |
| | | — | −18.5 (6.4) | 3.6 (14.7) |
| Cycle 11, Day 1 | Number Analyzed | 7 participants | 6 participants | 8 participants |
| | | 0.7 (4.3) | −5.2 (11.2) | −3.0 (19.7) |
| Cycle 12, Day 1 | Number Analyzed | 1 participants | 1 participants | 6 participants |
| | | −9.0 (—) | 0.0 (—) | −8.0 (14.2) |
| Cycle 13, Day 1 | Number Analyzed | 7 participants | 4 participants | 8 participants |
| | | −0.3 (7.1) | −10.3 (18.7) | −3.3 (17.6) |
| Cycle 14, Day 1 | Number Analyzed | 1 participants | 1 participants | 6 participants |
| | | 23.0 (—) | −7.0 (—) | −5.8 (16.5) |
| Cycle 15, Day 1 | Number Analyzed | 6 participants | 3 participants | 7 participants |
| | | −2.8 (10.8) | −1.0 (5.6) | −7.0 (12.0) |
| Cycle 16, Day 1 | Number Analyzed | 1 participants | 2 participants | 4 participants |
| | | −3.0 (—) | −14.0 (12.7) | −15.5 (9.5) |
| Cycle 17, Day 1 | Number Analyzed | 5 participants | 3 participants | 7 participants |
| | | −3.0 (6.2) | −17.0 (11.4) | −18.1 (15.6) |
| Cycle 18, Day 1 | Number Analyzed | 0 participants | 2 participants | 3 participants |
| | | — | −16.0 (8.5) | 0.0 (13.9) |
| Cycle 19, Day 1 | Number Analyzed | 5 participants | 3 participants | 4 participants |
| | | −4.6 (6.1) | −7.7 (10.5) | −7.0 (5.4) |
| Cycle 20, Day 1 | Number Analyzed | 0 participants | 2 participants | 3 participants |
| | | — | −9.5 (16.3) | −9.3 (15.4) |
| Cycle 21, Day 1 | Number Analyzed | 3 participants | 1 participants | 5 participants |
| | | 0.3 (9.3) | −18.0 (—) | −14.4 (24.0) |
| Cycle 22, Day 1 | Number Analyzed | 0 participants | 2 participants | 2 participants |
| | | — | −18.5 (6.4) | −21.0 (14.1) |
| Cycle 23, Day 1 | Number Analyzed | 3 participants | 2 participants | 4 participants |
| | | 0.0 (8.7) | −21.0 (19.8) | −2.8 (18.8) |
| Cycle 24, Day 1 | Number Analyzed | 1 participants | 1 participants | 1 participants |
| | | 0.0 (—) | −15.0 (—) | −34.0 (—) |
| Cycle 25, Day 1 | Number Analyzed | 2 participants | 2 participants | 3 participants |
| | | −3.0 (7.1) | −19.5 (13.4) | −6.3 (24.1) |
| Cycle 26, Day 1 | Number Analyzed | 1 participants | 0 participants | 0 participants |
| | | 0.0 (—) | — | — |
| Cycle 27, Day 1 | Number Analyzed | 2 participants | 2 participants | 2 participants |
| | | 5.5 (16.3) | −24.0 (12.7) | −14.5 (24.7) |
| Cycle 29, Day 1 | Number Analyzed | 2 participants | 2 participants | 2 participants |
| | | −1.0 (17.0) | −27.5 (21.9) | 12.0 (14.1) |
| Cycle 30, Day 1 | Number Analyzed | 1 participants | 0 participants | 0 participants |
| | | 7.0 (—) | — | — |
| Cycle 31, Day 1 | Number Analyzed | 2 participants | 1 participants | 1 participants |
| | | 7.5 (17.7) | −36.0 (—) | 17.0 (—) |
| Cycle 32, Day 1 | Number Analyzed | 1 participants | 0 participants | 0 participants |
| | | 1.0 (—) | — | — |
| Cycle 33, Day 1 | Number Analyzed | 2 participants | 1 participants | 0 participants |
| | | 14.0 (11.3) | −39.0 (—) | — |
| Cycle 34, Day 1 | Number Analyzed | 1 participants | 0 participants | 0 participants |
| | | 2.0 (—) | — | — |
| Cycle 35, Day 1 | Number Analyzed | 2 participants | 2 participants | 1 participants |
| | | 10.5 (7.8) | −23.5 (16.3) | 13.0 (—) |
| Cycle 36, Day 1 | Number Analyzed | 1 participants | 0 participants | 1 participants |
| | | 6.0 (—) | — | −6.0 (—) |
| Cycle 37, Day 1 | Number Analyzed | 1 participants | 1 participants | 0 participants |
| | | −7.0 (—) | −41.0 (—) | — |
| Cycle 38, Day 1 | Number Analyzed | 1 participants | 0 participants | 0 participants |
| | | 2.0 (—) | — | — |
| Cycle 39, Day 1 | Number Analyzed | 2 participants | 2 participants | 1 participants |
| | | 24.0 (28.3) | −30.0 (21.2) | 23.0 (—) |
| Cycle 40, Day 1 | Number Analyzed | 1 participants | 0 participants | 0 participants |
| | | 0.0 (—) | — | — |
| Cycle 41, Day 1 | Number Analyzed | 1 participants | 0 participants | 0 participants |
| | | 38.0 (—) | — | — |
| Cycle 43, Day 1 | Number Analyzed | 1 participants | 1 participants | 1 participants |
| | | 33.0 (—) | −16.0 (—) | 21.0 (—) |
| Cycle 47, Day 1 | Number Analyzed | 1 participants | 0 participants | 1 participants |
| | | 19.0 (—) | — | 25.0 (—) |
| Cycle 48, Day 1 | Number Analyzed | 1 participants | 0 participants | 0 participants |
| | | −13.0 (—) | — | — |
| Cycle 49, Day 1 | Number Analyzed | 1 participants | 0 participants | 1 participants |
| | | 5.0 (—) | — | 1.0 (—) |
| Cycle 51, Day 1 | Number Analyzed | 1 participants | 0 participants | 0 participants |
| | | 15.0 (—) | — | — |
| Cycle 52, Day 1 | Number Analyzed | 1 participants | 0 participants | 0 participants |
| | | 1.0 (—) | — | — |
| Cycle 53, Day 1 | Number Analyzed | 1 participants | 0 participants | 0 participants |
| | | 15.0 (—) | — | — |
| Cycle 54, Day 1 | Number Analyzed | 1 participants | 0 participants | 0 participants |
| | | 4.0 (—) | — | — |
| Cycle 55, Day 1 | Number Analyzed | 1 participants | 0 participants | 0 participants |
| | | 29.0 (—) | — | — |
| Cycle 56, Day 1 | Number Analyzed | 1 participants | 0 participants | 0 participants |
| | | 9.0 (—) | — | — |
| Cycle 57, Day 1 | Number Analyzed | 1 participants | 0 participants | 0 participants |
| | | 18.0 (—) | — | — |
| Cycle 58, Day 1 | Number Analyzed | 1 participants | 0 participants | 0 participants |
| | | 5.0 (—) | — | — |
| Cycle 60, Day 1 | Number Analyzed | 1 participants | 0 participants | 0 participants |
| | | −3.0 (—) | — | — |
| Cycle 61, Day 1 | Number Analyzed | 1 participants | 0 participants | 0 participants |
| | | 5.0 (—) | — | — |
| Cycle 62, Day 1 | Number Analyzed | 1 participants | 0 participants | 0 participants |
| | | −1.0 (—) | — | |
| Cycle 63, Day 1 | Number Analyzed | 1 participants | 0 participants | 0 participants |
| | | 12.0 (—) | — | — |
| Cycle 67, Day 1 | Number Analyzed | 1 participants | 0 participants | 0 participants |
| | | 12.0 (—) | — | — |
| EOT | Number Analyzed | 10 participants | 7 participants | 8 participants |
| | | 5.2 (27.5) | −2.4 (15.0) | −12.6 (20.1) |

Change From Baseline in Percent Predicted Residual Volume (RV) at Each Specified Time Points. RV is the volume of air remaining in the lungs after maximum forceful expiration. Baseline value was defined as valid and last non-missing value obtained within 28 days prior to participant receiving first study medication. EOT visit was performed within 3 days after the participant's last dose of study drug. Timeframe: Baseline, Day 1 of Cycles 2, 3, 4, 5, 6, 7, 8, 9, 10, 11, 12, 13, 14, 15, 16, 17, 18, 19, 20, 21, 22, 23, 24, 25, 26, 27, 29, 30, 31, 32, 33, 34, 35, 36, 37, 38, 39, 40, 41, 43, 47, 48, 49, 51, 52, 53, 54, 55, 56, 57, 58, 60, 61, 62, 63, 67, EOT (i.e., anytime up to 64.2 months). Analysis was performed on mITT population. Here, ‘number analyzed’=participants with available data for each specified category. Here, ‘0’ in the number analyzed field signifies that none of the participants were available for the analysis at the specified time points. The data is shown in the table below.

TABLE 26
Change From Baseline in Percent Predicted Residual Volume (RV) at
Each Specified Time Points

| | | Cohort 1: | Cohort 2: | Cohort 3: |
|---|---|---|---|---|
| | | Belumosudil | Belumosudil | Belumosudil |
| | | 200 mg QD | 200 mg BID | 400 mg QD |
| Overall Number of Participants Analyzed | | 17 | 16 | 21 |
| Change From Baseline | | | | |
| in Percent Predicted | | | | |
| Residual Volume (RV) | | | | |
| at Each Specified Time | | | | |
| Points | | | | |
| Mean (Standard Deviation) | | | | |
| Unit of measure: | | | | |
| percent predicted RV | | | | |
| Cycle 2, Day 1 | Number Analyzed | 0 participants | 0 participants | 4 participants |
| | | — | — | −18.0 (30.1) |
| Cycle 3, Day 1 | Number Analyzed | 14 participants | 12 participants | 12 participants |
| | | 0.2 (22.2) | −4.6 (18.8) | 9.0 (34.4) |
| Cycle 4, Day 1 | Number Analyzed | 0 participants | 0 participants | 6 participants |
| | | — | — | 17.8 (29.0) |
| Cycle 5, Day 1 | Number Analyzed | 13 participants | 11 participants | 10 participants |
| | | 5.2 (24.5) | −11.5 (26.9) | −3.7 (29.8) |
| Cycle 6, Day 1 | Number Analyzed | 0 participants | 0 participants | 7 participants |
| | | — | — | −10.9 (37.5) |
| Cycle 7, Day 1 | Number Analyzed | 11 participants | 8 participants | 12 participants |
| | | 4.9 (29.1) | −9.9 (17.1) | −7.5 (33.0) |
| Cycle 8, Day 1 | Number Analyzed | 0 participants | 1 participants | 6 participants |
| | | — | −28.0 ( —) | 2.5 (39.7) |
| Cycle 9, Day 1 | Number Analyzed | 9 participants | 8 participants | 10 participants |
| | | 6.4 (27.4) | −19.9 (24.3) | 0.1 (35.6) |
| Cycle 10, Day 1 | Number Analyzed | 0 participants | 2 participants | 5 participants |
| | | — | −47.5 (0.7) | 1.4 (38.2) |
| Cycle 11, Day 1 | Number Analyzed | 7 participants | 6 participants | 8 participants |
| | | 22.1 (29.6) | −10.5 (28.3) | 5.4 (38.0) |
| Cycle 12, Day 1 | Number Analyzed | 1 participants | 1 participants | 6 participants |
| | | −28.0 (—) | −5.0 (—) | −22.2 (49.9) |
| Cycle 13, Day 1 | Number Analyzed | 7 participants | 4 participants | 8 participants |
| | | 20.3 (19.9) | −27.0 (35.6) | 0.3 (32.6) |
| Cycle 14, Day 1 | Number Analyzed | 1 participants | 1 participants | 6 participants |
| | | 78.0 (—) | −31.0 (—) | −6.5 (36.3) |
| Cycle 15, Day 1 | Number Analyzed | 6 participants | 3 participants | 7 participants |
| | | 20.3 (27.4) | −0.7 (18.1) | −16.7 (22.3) |
| Cycle 16, Day 1 | Number Analyzed | 1 participants | 2 participants | 4 participants |
| | | 0.0 (—) | −36.5 (13.4) | −27.5 (31.9) |
| Cycle 17, Day 1 | Number Analyzed | 5 participants | 3 participants | 7 participants |
| | | 13.6 (27.8) | −47.3 (25.1) | −39.1 (26.2) |
| Cycle 18, Day 1 | Number Analyzed | 0 participants | 2 participants | 3 participants |
| | | — | −42.0 (0.0) | −7.3 (33.7) |
| Cycle 19, Day 1 | Number Analyzed | 5 participants | 3 participants | 4 participants |
| | | 5.4 (28.7) | −19.7 (18.0) | −23.5 (18.7) |
| Cycle 20, Day 1 | Number Analyzed | 0 participants | 2 participants | 3 participants |
| | | — | −7.5 (37.5) | −22.0 (44.3) |
| Cycle 21, Day 1 | Number Analyzed | 3 participants | 1 participants | 5 participants |
| | | 8.3 (10.0) | −44.0 (—) | −30.2 (51.2) |
| Cycle 22, Day 1 | Number Analyzed | 0 participants | 2 participants | 2 participants |
| | | — | −33.0 (19.8) | −41.0 (46.7) |
| Cycle 23, Day 1 | Number Analyzed | 3 participants | 1 participants | 4 participants |
| | | 0.3 (5.1) | −72.0 (—) | 1.5 (44.8) |
| Cycle 24, Day 1 | Number Analyzed | 1 participants | 1 participants | 1 participants |
| | | −4.0 (—) | −17.0 (—) | −78.0 (—) |
| Cycle 25, Day 1 | Number Analyzed | 2 participants | 2 participants | 3 participants |
| | | 1.0 (9.9) | −34.0 (31.1) | −23.3 (56.0) |
| Cycle 26, Day 1 | Number Analyzed | 1 participants | 0 participants | 0 participants |
| | | −3.0 (—) | — | — |
| Cycle 27, Day 1 | Number Analyzed | 2 participants | 2 participants | 2 participants |
| | | 19.5 (41.7) | −39.5 (20.5) | −41.0 (28.3) |
| Cycle 29, Day 1 | Number Analyzed | 2 participants | 1 participants | 2 participants |
| | | −13.0 (42.4) | −13.0 (—) | 17.5 (40.3) |
| Cycle 30, Day 1 | Number Analyzed | 1 participants | 0 participants | 0 participants |
| | | 21.0 (—) | — | — |
| Cycle 31, Day 1 | Number Analyzed | 2 participants | 1 participants | 1 participants |
| | | 15.0 (43.8) | −93.0 (—) | 61.0 (—) |
| Cycle 32, Day 1 | Number Analyzed | 1 participants | 0 participants | 0 participants |
| | | 5.0 (—) | — | — |
| Cycle 33, Day 1 | Number Analyzed | 2 participants | 1 participants | 0 participants |
| | | 14.5 (36.1) | −70.0 (—) | — |
| Cycle 34, Day 1 | Number Analyzed | 1 participants | 0 participants | 0 participants |
| | | −1.0 (—) | — | — |
| Cycle 35, Day 1 | Number Analyzed | 2 participants | 2 participants | 1 participants |
| | | 3.5 (36.1) | −34.5 (33.2) | 47.0 (—) |
| Cycle 36, Day 1 | Number Analyzed | 1 participants | 0 participants | 1 participants |
| | | 17.0 (—) | — | −20.0 (—) |
| Cycle 37, Day 1 | Number Analyzed | 1 participants | 1 participants | 0 participants |
| | | −54.0 (—) | −70.0 (—) | — |
| Cycle 38, Day 1 | Number Analyzed | 1 participants | 0 participants | 0 participants |
| | | 8.0 (—) | — | — |
| Cycle 39, Day 1 | Number Analyzed | 2 participants | 2 participants | 1 participants |
| | | 43.0 (89.1) | −43.0 (42.4) | 81.0 (—) |
| Cycle 40, Day 1 | Number Analyzed | 1 participants | 0 participants | 0 participants |
| | | −1.0 (—) | — | — |
| Cycle 41, Day 1 | Number Analyzed | 1 participants | 0 participants | 0 participants |
| | | 80.0 (—) | — | — |
| Cycle 43, Day 1 | Number Analyzed | 1 participants | 1 participants | 1 participants |
| | | 58.0 (—) | −26.0 (—) | 75.0 (—) |
| Cycle 47, Day 1 | Number Analyzed | 1 participants | 0 participants | 1 participants |
| | | 42.0 (—) | — | 87.0 (—) |
| Cycle 48, Day 1 | Number Analyzed | 1 participants | 0 participants | 0 participants |
| | | −50.0 (—) | — | — |
| Cycle 49, Day 1 | Number Analyzed | 1 participants | 0 participants | 1 participants |
| | | −14.0 (—) | — | −5.0 (—) |
| Cycle 51, Day 1 | Number Analyzed | 1 participants | 0 participants | 0 participants |
| | | 22.0 (—) | — | — |
| Cycle 52, Day 1 | Number Analyzed | 1 participants | 0 participants | 0 participants |
| | | −4.0 (—) | — | — |
| Cycle 53, Day 1 | Number Analyzed | 1 participants | 0 participants | 0 participants |
| | | 22.0 (—) | — | — |
| Cycle 54, Day 1 | Number Analyzed | 1 participants | 0 participants | 0 participants |
| | | 4.0 (—) | — | — |
| Cycle 55, Day 1 | Number Analyzed | 1 participants | 0 participants | 0 participants |
| | | 53.0 (—) | — | — |
| Cycle 56, Day 1 | Number Analyzed | 1 participants | 0 participants | 0 participants |
| | | 17.0 (—) | — | — |
| Cycle 57, Day 1 | Number Analyzed | 1 participants | 0 participants | 0 participants |
| | | 18.0 (—) | — | — |
| Cycle 58, Day 1 | Number Analyzed | 1 participants | 0 participants | 0 participants |
| | | 4.0 (—) | — | — |
| Cycle 60, Day 1 | Number Analyzed | 1 participants | 0 participants | 0 participants |
| | | −20.0 (—) | — | — |
| Cycle 61, Day 1 | Number Analyzed | 1 participants | 0 participants | 0 participants |
| | | −18.0 (—) | — | — |
| Cycle 62, Day 1 | Number Analyzed | 1 participants | 0 participants | 0 participants |
| | | −8.0 (—) | — | — |
| Cycle 63, Day 1 | Number Analyzed | 1 participants | 0 participants | 0 participants |
| | | 6.0 (—) | — | — |
| Cycle 67, Day 1 | Number Analyzed | 1 participants | 0 participants | 0 participants |
| | | 2.0 (—) | — | — |
| EOT | Number Analyzed | 11 participants | 8 participants | 8 participants |
| | | 6.3 (24.3) | 10.6 (34.9) | −18.5 (32.6) |

Pharmacokinetics (PK): Maximum Observed Plasma Concentration (Cmax) of Pharmacokinetics (PK): Maximum Observed Plasma Concentration (Cmax) of Belumosudil and Its Metabolites (KD025 ml and KD025m2). Cmax was the maximum observed plasma concentration, obtained by a non-compartmental analysis. Cmax data for Belumosudil and its metabolites KD025 ml and KD025m2 are reported in this outcome measure. Timeframe: Cycles 1 and 2: pre-dose (0 hour), 1, 2, 3, 4, 5, and 6 hours post-dose on Day 1. Analysis was performed on PK population which included all participants who received at least one dose of study drug and had at least 1 post-dose PK sample drawn. Here, ‘number analyzed’=participants with available data for each specified category. The data is shown in the table below.

TABLE 27
Pharmacokinetics (PK) Cmax

| | | Cohort 1: | Cohort 2: | Cohort 3: |
|---|---|---|---|---|
| | | Belumosudil | Belumosudil | Belumosudil |
| | | 200 mg QD | 200 mg BID | 400 mg QD |
| Overall Number of | | 17 | 16 | 21 |
| Participants Analyzed | | | | |
| Pharmaco- | | | | |
| kinetics | | | | |
| (PK): | | | | |
| Maximum | | | | |
| Observed | | | | |
| Plasma | | | | |
| Concentration | | | | |
| (Cmax) of | | | | |
| Belumosudil | | | | |
| and Its | | | | |
| Metabolites | | | | |
| (KD025m1 | | | | |
| and | | | | |
| KD025m2) | | | | |
| Geometric | | | | |
| Mean | | | | |
| (Geometric | | | | |
| Coefficient | | | | |
| of Variation) | | | | |
| Unit of | | | | |
| measure: | | | | |
| nanograms | | | | |
| per milliliter | | | | |
| Belumosudil: | Number | 16 | 16 | 21 |
| Cycle 1 | Analyzed | participants | participants | participants |
| Day 1 | | 2500 | 1400 | 2960 |
| | | (60.1%) | (98.5%) | (69.7%) |
| Belumosudil: | Number | 16 | 14 | 16 |
| Cycle 2 | Analyzed | participants | participants | participants |
| Day 1 | | 2020 | 1890 | 3270 |
| | | (101%) | (120%) | (63.0%) |
| KD025m1: | Number | 6 | 6 | 14 |
| Cycle 1 | Analyzed | participants | participants | participants |
| Day 1 | | 44.4 | 34.8 | 63.9 |
| | | (67.9%) | (58.8%) | (59.4%) |
| KD025m1: | Number | 7 | 5 | 11 |
| Cycle 2 | Analyzed | participants | participants | participants |
| Day 1 | | 36.2 | 45.0 | 55.8 |
| | | (43.0%) | (38.6%) | (82.1%) |
| KD025m2: | Number | 15 | 14 | 17 |
| Cycle 1 | Analyzed | participants | participants | participants |
| Day 1 | | 208 (93.1%) | 99.2 (112%) | 388 (82.3%) |
| KD025m2: | Number | 15 | 13 | 16 |
| Cycle 2 | Analyzed | participants | participants | participants |
| Day 1 | | 158 (150%) | 145 (147%) | 265 (154%) |

Pharmacokinetics (PK): Time of the Maximum Observed Plasma Concentration (Tmax) of Belumosudil and Its Metabolites (KD025 ml and KD025m2). Tmax was defined as time to reach maximum observed plasma concentration, obtained by a non-compartmental analysis. Tmax data for Belumosudil and its metabolites KD025 ml and KD025m2 are reported in this outcome measure. Timeframe: Cycles 1 and 2: pre-dose (0 hour), 1, 2, 3, 4, 5, and 6 hours post-dose on Day 1. Analysis was performed on PK population. Here, ‘number analyzed’=participants with available data for each specified category. The data is shown in the table below.

TABLE 28
Pharmacokinetics (PK) Tmax

| | | Cohort 1: | Cohort 2: | Cohort 3: |
|---|---|---|---|---|
| | | Belumosudil | Belumosudil | Belumosudil |
| | | 200 mg QD | 200 mg BID | 400 mg QD |
| Overall Number of | | 17 | 16 | 21 |
| Participants Analyzed | | | | |
| Pharmacokinetics: | | | | |
| Time of the | | | | |
| Maximum | | | | |
| Observed Plasma | | | | |
| Concentration | | | | |
| (Tmax) | | | | |
| of Belumosudil | | | | |
| and Its | | | | |
| Metabolites | | | | |
| (KD025m1 | | | | |
| and KD025m2) | | | | |
| Median | | | | |
| (Full Range) | | | | |
| Unit of | | | | |
| measure: hours | | | | |
| Belumosudil: | Number | 16 | 16 | 21 |
| Cycle 1 | Analyzed | participants | participants | participants |
| Day 1 | | 2.12 (0.98 to | 3.43 (1.97 to | 3.03 (1.85 to |
| | | 5.00) | 6.00) | 6.00) |
| Belumosudil: | Number | 16 | 14 | 16 |
| Cycle 2 | Analyzed | participants | participants | participants |
| Day 1 | | 2.53 (0 to | 2.47 (0 to | 2.66 (1.00 to |
| | | 4.87) | 5.83) | 6.02) |
| KD025m1: | Number | 6 | 6 | 14 |
| Cycle 1 | Analyzed | participants | participants | participants |
| Day 1 | | 2.50 (1.00 to | 1.75 (1.08 to | 1.98 (0.90 to |
| | | 3.98) | 5.95) | 4.95) |
| KD025m1: | Number | 7 | 5 | 11 |
| Cycle 2 | Analyzed | participants | participants | participants |
| Day 1 | | 2.98 (1.17 to | 2.05 (1.08 to | 2.17 (1.00 to |
| | | 3.03) | 5.83) | 4.03) |
| KD025m2: | Number | 15 | 14 | 17 |
| Cycle 1 | Analyzed | participants | participants | participants |
| Day 1 | | 2.82 (1.93 to | 3.00 (2.00 to | 2.98 (1.85 to |
| | | 5.00) | 6.00) | 6.00) |
| KD025m2: | Number | 15 | 13 | 16 |
| Cycle 2 | Analyzed | participants | participants | participants |
| Day 1 | | 2.98 (0 to | 2.00 (1.05 to | 3.03 (1.98 to |
| | | 3.93) | 5.07) | 6.02) |

Pharmacokinetics (PK): The Area Under the Plasma Concentration Versus Time Curve From Time 0 to 6 Hours Post-dose (AUC0-6 hr) of Belumosudil and Its Metabolites (KD025 ml and KD025m2). AUC0-6 hr was defined as area under the plasma concentration versus time curve from time 0 to 6 hours post-dose, obtained by a non-compartmental analysis from the concentration-time data. AUC0-6 hr data for Belumosudil and its metabolites KD025 ml and KD025m2 are reported in this outcome measure. Timeframe: Cycles 1 and 2: pre-dose (0 hour), 1, 2, 3, 4, 5, and 6 hours post-dose on Day 1. Analysis was performed on PK population. Here, ‘number analyzed’=participants with available data for each specified category. The data is shown in the table below.

TABLE 29
Pharmacokinetics (PK) AUC0-6 hr

| | | Cohort 1: | Cohort 2: | Cohort 3: |
|---|---|---|---|---|
| | | Belumosudil | Belumosudil | Belumosudil |
| | | 200 mg QD | 200 mg BID | 400 mg QD |
| Overall Number of | | 17 | 16 | 21 |
| Participants Analyzed | | | | |
| Pharmacokinetics: | | | | |
| The Area Under | | | | |
| the Plasma | | | | |
| Concentration | | | | |
| Versus | | | | |
| Time Curve | | | | |
| From Time | | | | |
| 0 to 6 Hours | | | | |
| Post-dose | | | | |
| (AUC0-6 hr) of | | | | |
| Belumosudil | | | | |
| and Its | | | | |
| Metabolites | | | | |
| (KD025m1 | | | | |
| and KD025m2) | | | | |
| Geometric Mean | | | | |
| (Geometric | | | | |
| Coefficient | | | | |
| of Variation) | | | | |
| Unit of | | | | |
| measure: hours* | | | | |
| nanograms per | | | | |
| milliliter | | | | |
| Belumosudil: | Number | 16 | 16 | 21 |
| Cycle 1 | Analyzed | participants | participants | participants |
| Day 1 | | 8350 | 4960 | 11500 |
| | | (60.3%) | (108%) | (97.3%) |
| Belumosudil: | Number | 16 | 14 | 16 |
| Cycle 2 | Analyzed | participants | participants | participants |
| Day 1 | | 7090 | 7190 | 12000 |
| | | (95.9%) | (115%) | (68.8%) |
| KD025m1: | Number | 6 | 6 | 14 |
| Cycle 1 | Analyzed | participants | participants | participants |
| Day 1 | | 140 (72.3%) | 114 (60.8%) | 216 (61.9%) |
| KD025m1: | Number | 7 | 5 | 11 |
| Cycle 2 | Analyzed | participants | participants | participants |
| Day 1 | | 123 (38.2%) | 160 (31.3%) | 178 (72.9%) |
| KD025m2: | Number | 15 | 14 | 17 |
| Cycle 1 | Analyzed | participants | participants | participants |
| Day 1 | | 565 (90.8%) | 282 (125%) | 1150 (97.1%) |
| KD025m2: | Number | 15 | 13 | 16 |
| Cycle 2 | Analyzed | participants | participants | participants |
| Day 1 | | 471 (145%) | 513 (122%) | 923 (144%) |

### Example 2: a Phase II Randomized Study of Belumosudil

### Subject Eligibility

Eligible subjects were allogeneic hematopoietic cell transplant recipients aged≥12 years with persistent cGVHD manifestations after receiving 2 to 5 prior systemic lines of therapy. Subjects were required to be receiving stable corticosteroid therapy for 2 weeks prior to screening and to have a Karnofsky or Lansky Performance Status Scale score≥60. Certain concurrent immunosuppressive medications were allowed because drug-drug interactions were not anticipated. Subjects were excluded if they had a relapse of their underlying malignancy, had a forced expiratory volume in 1 second (FEV1)≤39% or an NIH lung symptom score of 3, had developed posttransplant lymphoproliferative disease, had liver transaminases (aspartate aminotransferase [AST] or alanine transaminase [ALT])>3 times the upper limit of normal, had a total bilirubin>1.5 times the upper limit of normal for any reason, or were currently receiving ibrutinib.

### Study Design and Treatment

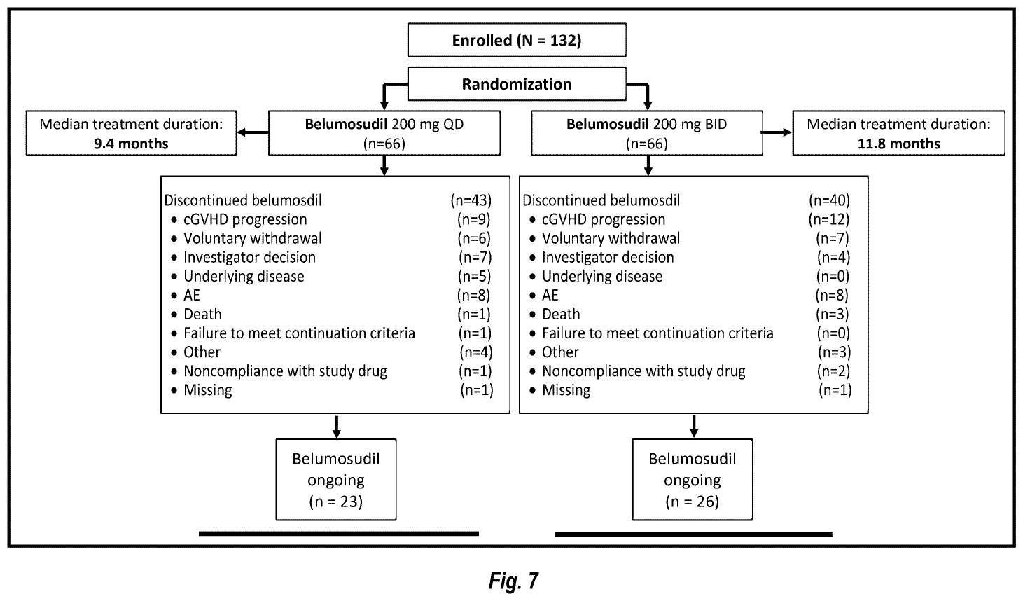

Screening for eligibility was conducted within 14 days of cycle 1 day 1. Treatment consisted of belumosudil 200 mg daily (Arm A) or 200 mg twice daily (Arm B) administered orally in subjects with cGVHD (FIG. 7). Randomization was stratified (1:1) by cGVHD severity and prior exposure to ibrutinib. Belumosudil was administered continuously in 28-day treatment cycles until clinically significant progression of cGVHD or unacceptable toxicity. Progression was defined using an organ-specific cGVHD response assessment, as defined by the 2014 NIH Consensus Development Project on Criteria for Clinical Trials in cGVHD, referred to as the 2014 NIH Consensus Criteria. After ≥2 weeks on belumosudil, corticosteriod therapy could be tapered at the discretion of the investigator. Subjects who did not achieve a response after 12 cycles of belumosudil treatment should be withdrawn if in the Investigator's judgment there is no evidence of clinical benefit.

### Study Endpoints

The primary end point was best ORR at any time, defined as the proportion of subjects who achieved complete response (CR) or partial response (PR) according to the 2014 NIH Consensus Criteria. All responses were assessed by the study site investigators. Secondary end points included duration of response (DOR), time to response, changes in LSS summary score, failure-free survival (FFS), corticosteroid dose reductions, and overall survival (OS). DOR was measured from the time of initial PR or CR until documented progression from best response of cGVHD, time from initial response to start of additional systemic cGVHD therapy, or death. The 7-day LSS summary score was calculated based on the developer recommendations and was compared with the score from baseline; an improvement≥7 points was considered clinically meaningful. FFS was defined as the interval between the start of belumosudil and the addition of a new cGVHD therapy, relapse, or NRM. The safety of belumosudil was evaluated by adverse event (AE) and serious AE (SAE) assessments. Relative dose intensity (RDI) was used as a surrogate measure of drug tolerability and was defined as actual dose intensity/planned dose intensity, where dose intensity was defined as the cumulative dose over the duration of exposure (mg/d). Actual dose intensity captured the sum of actual doses received over the duration of exposure and incorporated dose reductions and/or interruptions.

### Statistical Analysis

The sample size was based on the primary efficacy end point (best ORR), with 1 planned interim analysis and a target ORR of 55%. With a target sample size of 63 subjects per treatment arm and an estimated 10% dropout rate, each treatment arm was estimated to have about 90% power to yield a 95% confidence interval (CI) of ORR that excluded 30% as the lower bound. Based on consultation with key opinion leaders, a 30% ORR was considered clinically meaningful in this heavily pretreated population with cGVHD and unmet medical needs. The Hochberg procedure was used for multiplicity adjustment for the primary end point of best ORR. The primary analysis was conducted using the modified intent-to-treat (mITT) population, defined as randomized subjects who received ≥1 dose of belumosudil. Interim, primary, and follow-up analyses were planned at about 2, 6, and 12 months, respectively, after 126 subjects were enrolled in the mITT population. Here, we report data from the 12-month analysis. CI was calculated using the Clopper-Pearson interval (exact) method.

Results. Subject Characteristics

A total of 132 subjects were enrolled in the clinical study. Overall, baseline demographics and clinical characteristics were comparable across treatment arms (Table 5) At enrollment, the median subject age was 56 years (range, 21-77). The median time from cGVHD diagnosis to enrollment was 28 months (range, 2-162). Thirty-one percent of subjects had moderate cGVHD at screening, and 67% had severe cGVHD, based on the 2014 NIH Consensus Criteria; 52% had involvement of ≥4 organs. Thirty-six percent of subjects had lung involvement at baseline, with 38% of these subjects having an NIH lung symptom score of 2. Subjects were previously treated with a median of 3 systemic lines of therapy. Seventy-two percent of subjects (n=79) had cGVHD refractory to their last systemic lines of therapy, 34% (n=45) had previously received ibrutinib, 29% (n=38) had previously received ruxolitinib, and 72% (n=95) had received ≥3 prior lines of therapy. The baseline median corticosteroid dose was 0.2 mg/kg per day (range, 0.03-1.07) of prednisone equivalent. The baseline mean corticosteroid dose was 0.25 mg/kg per day (range, 0.03-1.07) of prednisone equivalent.

TABLE 30
Baseline demographics and clinical characteristics

| | | Belumosudil | |
|---|---|---|---|
| | Belumosudil | 200 mg twice | |
| | 200 mg daily | daily | Total |
| Characteristic | (n = 66) | (n = 66) | (N = 132) |
| Age, median (range), y | 53 (21-77) | 57 (21-77) | 56 (21-77) |
| Males | 42 (64) | 33 (50) | 75 (57) |
| Indication for transplant | | | |
| AML | 28 (42) | 25 (38) | 53 (40) |
| ALL | 7 (11) | 12 (18) | 19 (14) |
| MDS | 8 (12) | 5 (8) | 13 (10) |
| CML | 5 (8) | 3 (5) | 8 (6) |
| Myelofibrosis | 3 (5) | 2 (3) | 5 (4) |
| CLL | 2 (3) | 2 (3) | 4 (3) |
| Non-Hodgkin lymphoma | 3 (5) | 4 (7) | 7 (5) |
| and DLBCL | | | |
| Other | 7 (11) | 11 (17) | 18 (14) |
| Conditioning intensity | | | |
| Myeloablative | 41 (62) | 42 (64) | 83 (63) |
| Nonmyeloablative | 22 (33) | 22 (33) | 44 (33) |
| Unknown | 3 (5) | 2 (3) | 5 (4) |
| Stem cell source | | | |
| Peripheral blood | 57 (86) | 63 (96) | 120 (91) |
| Bone marrow | 6 (9) | 3 (5) | 9 (7) |
| Cord blood | 0 | 0 | 0 |
| Unknown | 3 (5) | 0 | 3 (2) |
| HLA matching of | | | |
| donor/recipient | | | |
| Matched | 57 (86) | 62 (94) | 119 (90) |
| Partially matched | 8 (12) | 3 (5) | 11 (8) |
| Unknown | 0 | 1 (2) | 1 (1) |
| Missing | 1 (2) | 0 | 1 (1) |
| CMV-positive serostatus | | | |
| (donor/recipient) | | | |
| +/+ | 23 (35) | 16 (24) | 39 (30) |
| i) +/− | 3 (5) | 8 (12) | 11 (8) |
| ii) −/+− | 18 (27) | 17 (26) | 35 (27) |
| iii) −/− | 13 (20) | 16 (24) | 29 (22) |
| 1 unknown | 3 (5) | 3 (5) | 6 (5) |
| Unknown/unknown | 5 (8) | 6 (9) | 11 (8) |
| Missing | 1 (2) | 0 | 1 (1) |
| iv) Time from | 25 (2-162) | 30 (4-144) | 29 (2-162) |
| cGVHD diagnosis to | | | |
| enrollment, median | | | |
| (range), mo | | | |
| NIH cGVHD severity* | | | |
| Severe | 46 (70) | 43 (65) | 89 (67) |
| Moderate | 18 (27) | 23 (35) | 41 (31) |
| Mild | 2 (3) | 0 | 2 (2) |
| Organ involvement | | | |
| No. of organs involved, | 4 (0-7) | 4 (2-7) | 4 (0-7) |
| median (range) | | | |
| ≥4 organs involved | 33 (50) | 35 (53) | 68 (52) |
| Skin | 55 (83) | 55 (83) | 110 (83) |
| Joints/fascia | 51 (77) | 49 (74) | 100 (76) |
| Eyes | 48 (73) | 49 (74) | 97 (74) |
| Mouth | 30 (46) | 42 (64) | 42 (64) |
| Lungs | 24 (36) | 23 (35) | 47 (36) |
| Esophagus | 19 (29) | 12 (18) | 31 (24) |
| Upper GI | 13 (20) | 10 (15) | 23 (17) |
| Lower GI | 6 (9) | 7 (11) | 13 (10) |
| Liver | 9 (14) | 4 (6) | 13 (10) |
| Baseline global | | | |
| severity rating | | | |
| 0 | 1 (2) | 0 | 1 (1) |
| 1 | 0 | 0 | 0 |
| 2 | 2 (3) | 1 (2) | 3 (2) |
| 3 | 3 (5) | 2 (3) | 5 (4) |
| 4 | 8 (12) | 3 (5) | 11 (8) |
| 5 | 6 (9) | 8 (12) | 14 (11) |
| 6 | 12 (18) | 14 (21) | 26 (20) |
| 7 | 11 (17) | 20 (30) | 31 (24) |
| 8 | 19 (29) | 14 (21) | 33 (25) |
| 9 | 4 (6) | 3 (5) | 7 (5) |
| 10 | 0 | 1 (2) | 1 (1) |
| Median Karnofsky | | | |
| Performance Status | | | |
| 60-70 | 10 (15) | 19 (29) | 29 (22) |
| 80-90 | 52 (79) | 43 (65) | 95 (72) |
| 100 | 4 (6) | 4 (6) | 8 (6) |
| Prior therapy | | | |
| characteristics | | | |
| Median prior LOTs, n | 3 | 4 | 3 |
| 2 prior LOTs | 23 (35) | 14 (21) | 37 (28) |
| 3 prior LOTs | 13 (20) | 17 (26) | 30 (23) |
| 4 prior LOTs | 15 (23) | 14 (21) | 29 (22) |
| 5 prior LOTs | 14 (21) | 19 (29) | 33 (25) |
| ≥6 prior LOTs | 1 (2) | 2 (3) | 3 (2) |
| Refractory to prior LOT | 44 (79) | 35 (65) | 79 (72) |
| Prior systemic | | | |
| cGVHD therapy type | | | |
| CS (prednisone) | 65 (99) | 65 (99) | 130 (99) |
| Tacrolimus | 40 (61) | 42 (64) | 82 (62) |
| ECP | 31 (47) | 32 (49) | 63 (48) |
| Sirolimus | 29 (44) | 33 (50) | 62 (47) |
| Ibrutinib | 22 (33) | 23 (35) | 45 (34) |
| Ruxolitinib | 20 (30) | 18 (27) | 38 (29) |
| MMF | 18 (27) | 15 (23) | 33 (25) |
| Rituximab | 15 (23) | 13 (20) | 28 (21) |
| MTX | 3 (5) | 3 (5) | 6 (5) |
| Cyclosporine | 4 (6) | 3 (5) | 5 (4) |
| Imatinib | 3 (5) | 1 (2) | 4 (3) |
| Ixazomib | 0 | 1 (2) | 1 (1) |
| Ofatumumab | 0 | 1 (2) | 1 (1) |
| Concomitant systemic | | | |
| cGVHD therapy type† | | | |
| CS | 65 (99) | 66 (100) | 131 (99) |
| CNI | 24 (36) | 25 (38) | 49 (37) |
| ECP | 17 (26) | 22 (33) | 39 (30) |
| Sirolimus | 17 (26) | 18 (27) | 35 (27) |
| MMF | 11 (17) | 2 (3) | 13 (10) |
| Imatinib | 1 (2) | 1 (2) | 2 (2) |
| Rituximab | 1 (2) | 0 | 1 (1) |
| Ruxolitinib | 1 (2) | 0 | 1 (1) |
| Other systemic | 9 (14) | 13 (20) | 22 (17) |
| cGVHD therapies | | | |
| Prednisone- | 0.20 | 0.20 | 0.20 |
| equivalent dose at | (0.03-0.95) | (0.03-1.07) | (0.03-1.07) |
| enrollment, median | | | |
| (range), mg/kg/d | | | |

Unless otherwise noted, data are n (%). Percentages may not add to 100% because of rounding.
ALL, acute lymphocytic leukemia;
AML, acute myelogenous leukemia;
CLL, chronic lymphocytic leukemia;
CML, chronic myelogenous leukemia;
CMV, cytomegalovirus;
DLBCL, diffuse large B-cell lymphoma;
GI, gastrointestinal;
MDS, myelodysplastic syndrome;
MMF, mycophenolate mofetil;
MTX, methotrexate.
*Disease severity was determined using NIH Global Severity of cGVHD scoring.
†Classified as concomitant systemic cGVHD medications on cycle 1 day 1.

Unless otherwise noted, data are n (%). Percentages may not add to 100% because of rounding. *Disease severity was determined using NIH Global Severity of cGVHD scoring.†Classified as concomitant systemic cGVHD medications on cycle 1 day 1.
ALL, acute lymphocytic leukemia; AML, acute myelogenous leukemia; CLL, chronic lymphocytic leukemia; CML, chronic myelogenous leukemia; CMV, cytomegalovirus; DLBCL, diffuse large B-cell lymphoma; GI, gastrointestinal; MDS, myelodysplastic syndrome; MMF, mycophenolate mofetil; MTX, methotrexate.

The CONSORT diagram (FIG. 7) shows subject disposition. The median duration of treatment was 10 months (range, 0.4-22.0), and the median follow-up was 14 months (range, 1-22). Forty-four percent of subjects had received treatment for ≥12 months. At the time of the data analysis, 37% of subjects continued to receive belumosudil. Reasons for discontinuation included progression of cGVHD (n=21), voluntary withdrawal (n=13), AEs (n=16), physician decision (n=11), progression of underlying malignancy (n=5), death due to underlying malignancy or disease progression (n=4), other (n=7), and nonadherence to study drug (n=3).

### Efficacy

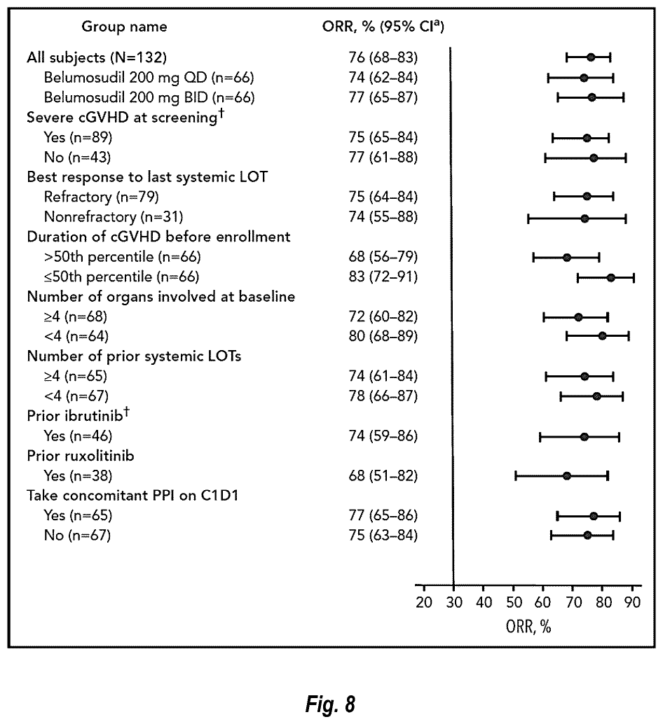

The best ORR for belumosudil 200 mg daily and 200 mg twice daily was 74% (95% CI, 62-84) and 77% (95% CI, 65-87), respectively (Table 6). High ORRs (61-85%) were observed in all subgroups (FIG. 8). Pooled responses across arms, unless stated otherwise. Efficacy of belumosudil was maintained, irrespective of prior ibrutinib (n=46) or ruxolitinib (n=38) therapy. The ORR for the subgroup with prior ruxolitinib therapy was 68% (95% CI, 51-83). The ORR (95% CI) for the subgroup with prior ibrutinib therapy was 74% (95% CI, 59-86).

TABLE 31
Efficacy end points in both arms within mITT population

| | Belumosudil, | Belumosudil, | |
|---|---|---|---|
| | 200 mg | 200 mg | |
| | daily | twice daily | Total |
| Efficacy end point | (n = 66) | (n = 66) | (N = 132) |
| ORR | 49 (74) | 51 (77) | 100 (76) |
| 95% Cl | 62-84 | 65-87 | 68-83 |
| ORR for | 47 (71) | 48 (73) | 95 (72) |
| responses occurring | | | |
| within 6 mo of treatment | | | |
| 95% Cl | 59-82 | 60-83 | 64-80 |
| CR | 2 (3) | 1 (2) | 3 (2) |
| PR | 45 (68) | 47 (71) | 92 (70) |
| ORR for | 49 (74) | 50 (76) | 99 (75) |
| responses occurring | | | |
| within 12 mo of treatment | | | |
| 95% Cl | 62-84 | 64-86 | 67-82 |
| CR | 4 (6) | 2 (3) | 6 (5) |
| PR | 45 (68) | 48 (73) | 93 (71) |
| Clinically significant | | | |
| improvement from | | | |
| baseline (LSS)* | | | |
| Overall | 39 (59) | 41 (62) | 80 (61) |
| Responder, n/N (%) | 34/49 (69) | 36/51 (71) | 70/100 (70) |
| Nonresponder, n/N (%) | 5/17 (29) | 5/15 (33) | 10/32 (31) |
| FFS at 6 mo (95% CI), % | 73 (61-83) | 76 (63-84) | 75 (66-81) |
| FFS at 12 mo | 57 (44-68) | 56 (43-67) | 56 (47-64) |
| (95% CI), % | | | |
| Proportion with | 42 (64) | 44 (67) | 86 (65) |
| CS reduction | | | |
| Median CS reduction | 38 | 50 | 50 |
| from baseline to greatest | | | |
| reduction, % | | | |
| Mean change in CS | | | |
| dose from baseline, % | | | |
| Overall | −43 | −48 | −45 |
| Responder | −49 | −58 | −54 |
| Nonresponder | −22 | −10 | −16 |
| CS discontinuation | 13 (20) | 15 (23) | 28 21) |

Unless otherwise noted, data are n (%).
*Changes in cGVHD symptom burden were measured using LSS.
Clinically meaningful improvement in symptom burden was defined as a decrease ≥ 7 points in LSS score.

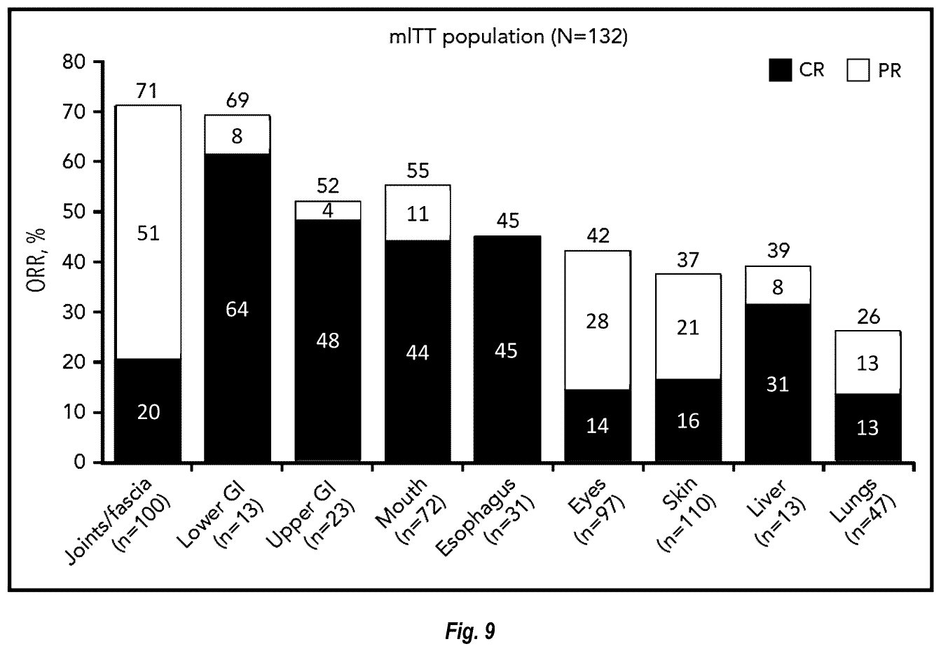

Best ORR, including CR, was evaluated across all affected organs. In the mITT population, organ-specific analyses demonstrated a best ORR of 37% in the skin, 42% in the eyes, 55% in the mouth, 39% in the liver, 26% in the lungs, 71% in the joints/fascia, 52% in the upper gastrointestinal (GI) tract, 69% in the lower GI tract, and 45% in the esophagus (FIG. 9; Table 7). Overall, 7 subjects achieved CR in all affected organs. Of the 12 subjects with lung responses, 3 were scored as CR based on normalization of FEV1 (median increase, 23%; range, 18-25), with an additional 3 CRs based on a reduction in NIH lung symptom score from 1 to 0 in the absence of pulmonary function tests. Six additional subjects had PR, with a ≥10% increase in FEV1 (median increase for all subjects achieving PR, 10%; range, 0-15) or a reduction in NIH lung symptom score of 1 point when pulmonary function tests were unavailable. Of the 41 subjects with skin responses, 11 had a decrease in sclerotic features, 15 had a decrease in body surface area involvement, and 13 had improvements in body surface area involvement and sclerotic features. Two subjects had skin responses according to the investigator's clinical assessment, not according to the 2014 NIH Consensus Criteria.

TABLE 32
Summary of ORR by dose and organ system

| | Belumosudil, | Belumosudil, | |
|---|---|---|---|
| Organ system, | 200 mg QD | 200 mg BID | Total |
| n (%) | (n = 66) | (n = 66) | (N = 132) |
| Joints and fascia | 51 (77) | 49 (74) | 100 (76) |
| CR | 10 (20) | 10 (20) | 20 (20) |
| PR | 28 (55) | 23 (47) | 51 (51) |
| ORR | 38 (75) | 33 (67) | 71 (71) |
| Lower GI | 6 (9) | 7 (11) | 13 (10) |
| CR | 4 (67) | 4 (57) | 8 (62) |
| PR | 0 | 1 (14) | 1 (8) |
| ORR | 4 (67) | 5 (71) | 9 (69) |
| Mouth | 30 (45) | 42 (64) | 72 (55) |
| CR | 15 (50) | 17 (41) | 32 (44) |
| PR | 1 (3) | 7 (17) | 8 (11) |
| ORR | 16 (53) | 24 (57) | 40 (56) |
| Upper GI | 13 (20) | 10 (15) | 23 (17) |
| CR | 7 (54) | 4 (40) | 11 (48) |
| PR | 1 (8) | 0 | 1 (4) |
| ORR | 8 (62) | 4 (40) | 12 (52) |
| Esophagus | 19 (29) | 12 (18) | 31 (23) |
| CR | 9 (47) | 5 (42) | 14 (45) |
| PR | 0 | 0 | 0 |
| ORR | 9 (47) | 5 (42) | 14 (45) |
| Eyes | 48 (73) | 49 (74) | 97 (73) |
| CR | 8 (17) | 6 (12) | 14 (14) |
| PR | 8 (17) | 19 (39) | 27 (28) |
| ORR | 16 (33) | 25 (51) | 41 (42) |
| Liver | 9 (14) | 4 (6) | 13 (10) |
| CR | 2 (22) | 2 (50) | 4 (31) |
| PR | 1 (11) | 0 | 1 (8) |
| ORR | 3 (33) | 2 (50) | 5 (39) |
| Skin | 55 (83) | 55 (83) | 110 (83) |
| CR | 8 (15) | 10 (18) | 18 (16) |
| PR | 10 (18) | 13 (24) | 23 (21) |
| ORR | 18 (33) | 23 (42) | 41 (37) |
| Lungs | 24 (36) | 23 (35) | 47 (36) |
| CR | 4 (17) | 2 (9) | 6 (13) |
| PR | 3 (13) | 3 (13) | 6 (13) |
| ORR | 7 (29) | 5 (22) | 12 (26) |

BID, twice a day;
CR, complete response;
GI, gastrointestinal;
ORR, overall response rate;
PR, partial response;
QD, every day.

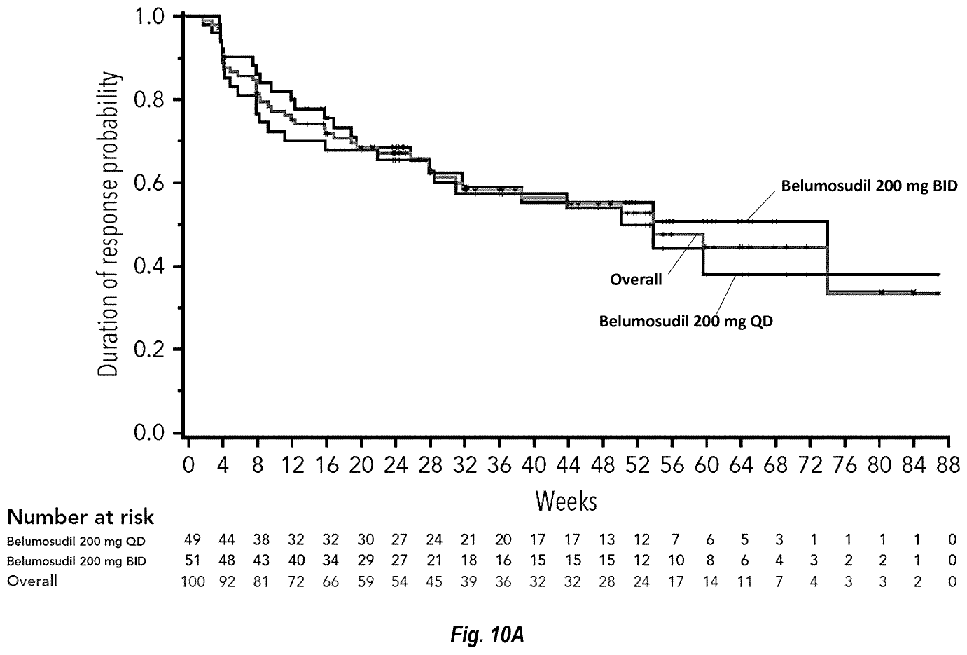

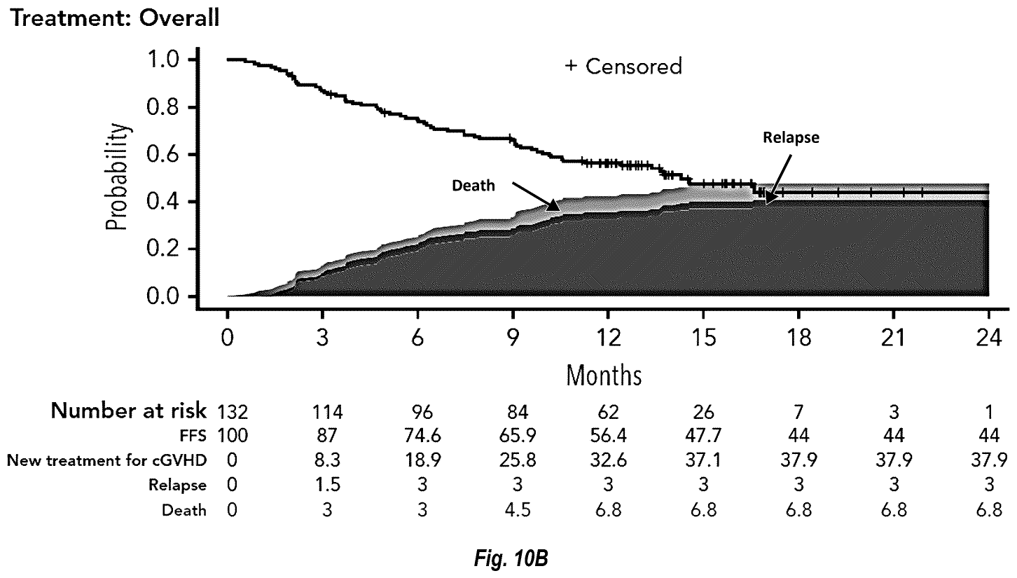

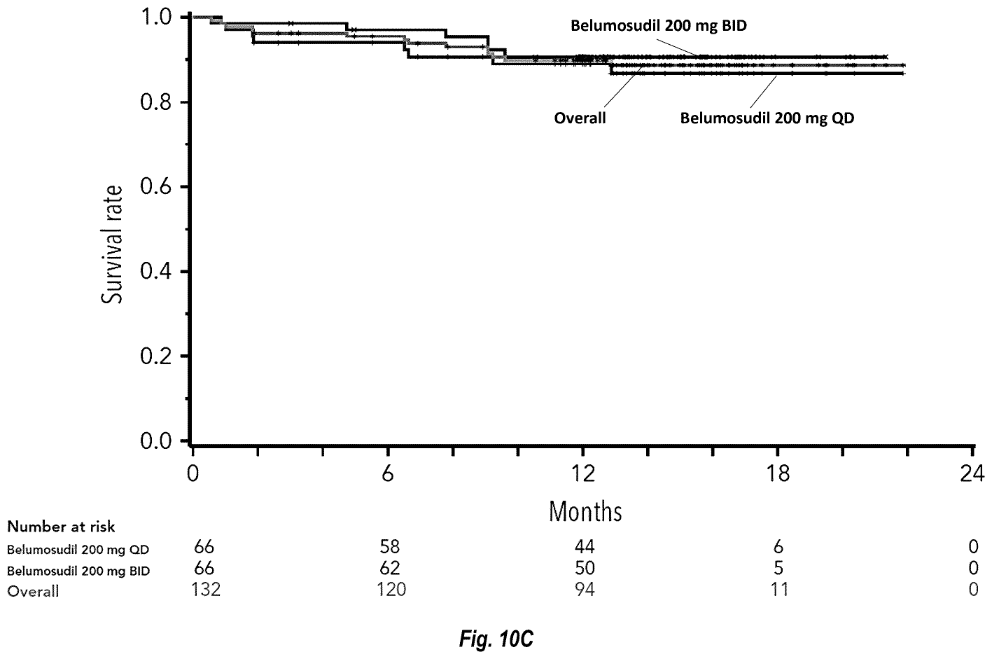

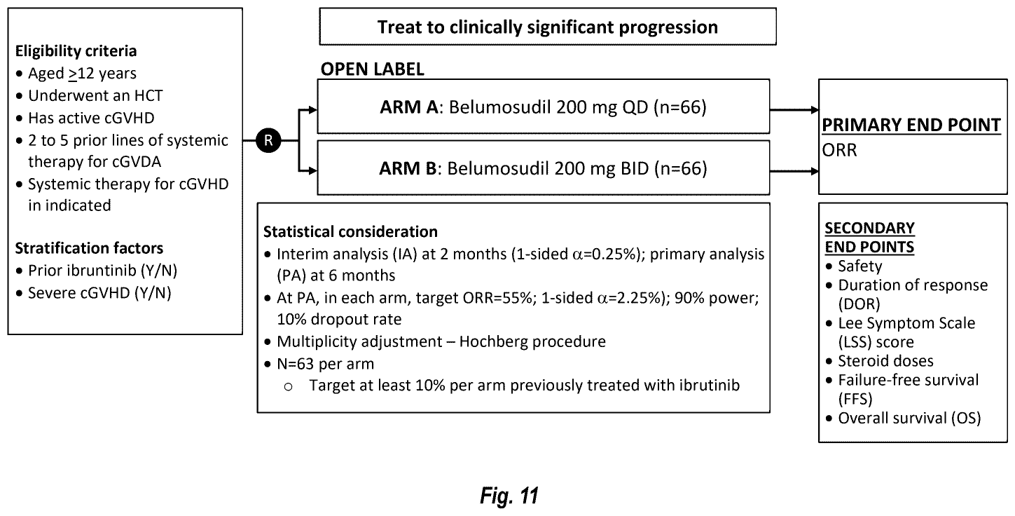

The overall median time to response was 5 weeks (range, 4-66) (FIG. 10A). Ninety-one percent of responses occurred within 6 months of treatment, with the remaining 9% of responses seen after 6 to 12 months of treatment. Fifty-nine percent of responders maintained responses for ≥20 weeks. The median DOR was 54 weeks in the responder population. The overall FFS rate was 75% (95% CI, 66-81) and 56% (95% CI, 47-64) at 6 and 12 months, respectively (FIG. 10B). Overall, low rates of nonrelapse mortality (NRM) (7%) and relapse (3%) were observed. The most common failure event was the initiation of a new systemic cGVHD therapy (38%). The 2-year OS rate was 89% (95% CI, 82-93) (FIG. 10C).

During treatment with belumosudil, 65% of subjects reduced their corticosteroid dose. The mean corticosteroid dose was reduced by 45% in the mITT population, with a mean corticosteroid dose reduction of 54% in responders. Twenty-one percent of subjects discontinued corticosteroid therapy. In addition, 22% of those subjects successfully discontinued calcineurin inhibitor (CNI) therapy, and 20% and 21% of subjects discontinued sirolimus and mycophenolate mofetil, respectively.

Clinically meaningful improvement (≥7-point reduction) in 7-day LSS summary score from baseline with belumosudil 200 mg daily and 200 mg twice daily was observed in 59% and 62% of them ITT population, respectively. This improvement was observed in 69% and 71% of responders in the belumosudil 200 mg daily and 200 mg twice-daily arms, respectively, as well as in 29% and 33% of nonresponders, respectively.

### Safety

Belumosudil was well tolerated, with a median RDI of 99.7%. Eighty-one percent of subjects received an RDI>95%. AEs were consistent with those expected in patients with cGVHD receiving corticosteroid therapy and other immunosuppressive therapies (ISTs) (Table 8). Thirty eight percent of subjects had ≥1 SAE; the most common was pneumonia (7%). The most common (≥5%) grade 3 or 4 AEs were pneumonia (8%), hypertension (6%), and hyperglycemia (5%). Twenty-four percent of subjects had increased liver function tests (LFTs); at baseline, 5% of subjects had increased g-glutamyltransferase (GGT), 5% of subjects had increased AST, 3% of subjects had increased ALT, 3% of subjects had increased LFTs, and 1% of subjects had increased bilirubin. The most common liver-related AE was increased GGT (12%). Of the 83 subjects who discontinued treatment, 28 (21%) discontinued because of overall AEs, 16 (12%) discontinued because of possible drug-related AEs, 5 (4%) discontinued because of progression of underlying malignant disease, and 21 (16%) discontinued because of progression of cGVHD. Fourteen subjects died during the study; 2 from multiorgan failure and infection possibly related to belumosudil, 2 from cardiac arrest, 2 from respiratory failure, 1 from hemothorax resulting from lung biopsy, 1 from acute myeloid leukemia recurrence, and 6 during long-term follow-up (LTFU) (>28 days after last dose). Grade≥3 anemia was reported in 3% of subjects, neutropenia was reported in 2% of subjects, and thrombocytopenia was reported in 2% of subjects. There was 1 case of Epstein-Barr viremia that required treatment and 1 case of cytomegalovirus (CMV) reactivation; both were unrelated to belumosudil treatment.

TABLE 33
Safety overview

| | Belumosudil, | Belumosudil, | |
|---|---|---|---|
| | 200 mg | 200 mg | |
| | daily | twice daily | Total |
| AE | (n = 66) | (n = 66) | (N = 132) |
| Any AE | 65 (99) | 66 (100) | 131 (99) |
| Grade ≥ 3 AEs | 37 (56) | 34 (52) | 71 (54) |
| Drug-related AEs | 49 (74) | 40 (61) | 89 (67) |
| SAEs | 27 (41) | 23 (35) | 50 (38) |
| Deaths* | 8 (12) | 6 (9) | 14 (11) |
| Drug-related SAEs | 5 (8) | 2 (3) | 7 (5) |
| All grades in ≥ 20% | | | |
| of subjects (overall) | | | |
| Fatigue | 30 (46) | 20 (30) | 50 (38) |
| Diarrhea | 23 (35) | 21 (32) | 44 (33) |
| Nausea | 23 (35) | 18 (27) | 41 (31) |
| Cough | 20 (30) | 17 (26) | 37 (28) |
| Upper respiratory | 17 (26) | 18 (27) | 35 (27) |
| tract infection | | | |
| Dyspnea | 21 (32) | 12 (18) | 33 (25) |
| Headache | 13 (20) | 18 (27) | 31 (24) |
| Peripheral edema | 17 (26) | 13 (20) | 30 (23) |
| Vomiting | 18 (27) | 10 (15) | 28 (21) |
| Muscle spasms | 13 (20) | 13 (20) | 26 (20) |
| Grade ≥ 3 in ≥ 5% of | | | |
| subjects in either arm | | | |
| Pneumonia | 6 (9) | 4 (6) | 10 (8) |
| Hypertension | 4 (6) | 4 (6) | 8 (6) |
| Hyperglycemia | 3 (5) | 3 (5) | 6 (5) |
| Liver-related AEs | 12 (18) | 19 (29) | 31 (24) |
| GGT increased | 6 (9) | 10 (15) | 16 (12) |
| AST increased | 5 (8) | 8 (12) | 13 (10) |
| ALT increased | 4 (6) | 7 (11) | 11 (8) |
| Blood alkaline phosphatase | 4 (6) | 6 (9) | 10 (8) |
| increased | | | |
| Hypoalbuminemia | 2 (3) | 2 (3) | 4 (3) |
| Transaminases increased | 1 (2) | 1 (2) | 2 (2) |
| Bilirubin conjugated | 1 (2) | 0 | 1 (1) |
| increased | | | |
| LFT increased | 1 (2) | 0 | 1 (1) |

All data are n (%).
*Six subjects died during long-term follow-up (LTFU) (>28 days after last dose).

The study of the present Example 2 demonstrated promising efficacy and a favorable safety profile for belumosudil therapy in patients with steroid-refractory (SR) cGVHD. The study population, consisting of subjects with severe cGVHD with multiorgan involvement and fibrotic manifestations who were treated after a median of 3 prior systemic lines of therapy, achieved best ORRs of 74% and 77% in the 200-mg daily and 200-mg twice-daily treatment arms, respectively.

Responses to belumosudil were sustained and clinically meaningful, regardless of response to prior treatment, severity of cGVHD, and number of organs involved. Responses were observed in all organs, which was clinically significant because CR and PR were achieved in difficult-to-treat organs, such as the lungs and liver, as well as in organs with fibrotic manifestations, such as the skin. cGVHD greatly impairs quality of life, especially in patients with fibrotic multiorgan involvement, which can be challenging to treat. The CR and PR observed, along with improvements in LSS, limited interactions, and lack of drug toxicities, are promising results that demonstrate that belumosudil treatment may have the potential to improve overall patient well-being. Seven subjects achieved CR in all affected organs. CR in all affected organs can be difficult to achieve in cGVHD because of the irreversible changes that occur in several organs, most notably the eyes and the lungs. The clinical benefit and tolerability of belumosudil therapy demonstrate the potential to halt the expected cycling of therapies for cGVHD seen in clinical practice. Responses were sustained in 59% of responders for ≥20 weeks at the 12-month analysis. The median DOR was 54 weeks in responders at the 12-month analysis.

In a patient population that is vulnerable to AEs and infections from immunosuppressive therapy (IST), belumosudil was well tolerated, allowing most subjects to remain on therapy to achieve clinically meaningful results and improvement in quality of life, which could be maintained with continued treatment. Only 12% of subjects discontinued belumosudil because of possible drug-related AEs. The median duration of treatment was 10 months (range, 0.4-22.0), and 37% of subjects continued to receive belumosudil after this time point., AEs were manageable, with few grade≥3 SAEs attributable to belumosudil. The SAE rates were comparable between the two treatment arms. Many current cGVHD treatment options are immunosuppressive and, consequently, increase the risk of infection and may cause hematologic toxicities, including leukopenia, anemia, and thrombocytopenia. Grade≥3 cytopenias were present in <4% of subjects, and there was only one report of cytomegalovirus (CMV) reactivation that was unrelated to belumosudil treatment. Cytopenias and CMV infection present as serious complications of cGVHD and cGVHD therapeutics; thus, the low rates of grade≥3 cytopenias and CMV infection rates are promising features of the safety profile of belumosudil.

### Recommended Dosage

The recommended dose of belumosudil is 200 mg given orally once daily until progression of chronic GVHD that requires new systemic therapy.

Instruct the patient on the following: Swallow belumosudil tablets whole with a glass of water. Do not cut, crush, or chew tablets. Take belumosudil with a meal at approximately the same time each day. If a dose of belumosudil is missed, it should be taken as soon as possible on the same day with a return to the normal schedule the following day. Instruct the patient to not take extra doses to make up the missed dose.

Treatment with belumosudil has not been studied in patients with pre-existing severe renal or hepatic impairment. For patients with pre-existing severe renal or hepatic impairment, consider the risks and potential benefits before initiating treatment with belumosudil.

### Dose Modifications for Adverse Reactions

Monitor total bilirubin, aspartate aminotransferase (AST), and alanine aminotransferase (ALT) at least monthly. Modify the belumosudil dosage for adverse reactions as per Table 34.

TABLE 34
Recommended Dosage Modifications for belumosudil for
Adverse Reactions

| Adverse | | Belumosudil |
|---|---|---|
| Reaction | Severity* | Dosage Modifications |
| Hepatotoxicity | Grade 3 AST | Hold belumosudil until recovery |
| | or ALT (5× to | of bilirubin, AST and ALT to |
| | 20× ULN) or | Grade 0-1, then resume |
| | Grade 2 bilirubin | belumosudil at the |
| | (1.5× to 3× | recommended dose. |
| | ULN) | |
| | Grade 4 | Discontinue belumosudil |
| | AST or ALT | permanently. |
| | (more than 20× | |
| | ULN) or | |
| | Grade ≥ 3 | |
| | bilirubin (more | |
| | than 3× ULN) | |
| Other adverse | Grade 3 | Hold belumosudil until recovery |
| reactions | | to Grade 0-1, then |
| | | resume belumosudil at |
| | | the recommended dose level. |
| | Grade 4 | Discontinue belumosudil |
| | | permanently. |

*Based on CTCAE v 4.03

### Dosage Modification Due to Drug Interactions

Strong CYP3A Inducers: Increase the dosage of belumosudil to 200 mg twice daily when coadministered with strong CYP3A inducers.

Proton Pump Inhibitors: Increase the dosage of belumosudil to 200 mg twice daily when coadministered with proton pump inhibitors.

In the present study, all subjects received belumosudil. Requiring randomization to best available therapy was not deemed appropriate, because subjects had previously progressed following ≥2 systemic lines of therapy, where response rates were historically low. Indeed, subjects in this study had already attempted a median of three prior lines of best available therapy for cGVHD before enrollment, with the use of ECP (48%), ibrutinib (34%), ruxolitinib (29%), and rituximab (21%), among other agents. The best ORR was 75% in subjects who were refractory to their last lines of therapy.

Based on the similar efficacy and safety observed in this study, 200 mg daily is the preferred dosage for the treatment of SR cGVHD. Although the 200-mg twice-daily dose showed higher responses in certain organs, such as the skin, and slightly fewer AEs, the difference compared with the 200-mg daily dose was not deemed significant.

In the two clinical trials of Examples 1 and 2, 83 adult patients with chronic GVHD were treated with belumosudil 200 mg once daily. The median duration of treatment was 9.2 months (range 0.5 to 44.7 months). Fatal adverse reaction was reported in one patient with severe nausea, vomiting, diarrhea and multi-organ failure.

Permanent discontinuation of belumosudil due to adverse reactions occurred in 18% of patients. The adverse reactions which resulted in permanent discontinuation of belumosudil in >3% of patients included nausea (4%). Adverse reactions leading to dose interruption occurred in 29% of patients. The adverse reactions leading to dose interruption in ≥2% were infections (11%), diarrhea (4%), and asthenia, dyspnea, hemorrhage, hypotension, liver function test abnormal, nausea, pyrexia, edema, and renal failure with (2% each).

The most common (≥20%) adverse reactions, including laboratory abnormalities, were infections, asthenia, nausea, diarrhea, dyspnea, cough, edema, hemorrhage, abdominal pain, musculoskeletal pain, headache, phosphate decreased, gamma glutamyl transferase increased, lymphocytes decreased, and hypertension.

Table 35 summarizes the nonlaboratory adverse reactions.

TABLE 35
Nonlaboratory Adverse Reactions in ≥ 10% Patients
with Chronic GVHD Treated with Belumosudil

| | Belumosudil | |
|---|---|---|
| | 200 mg once daily | |
| | (N = 83) | |
| Adverse Reaction | All Grades (%) | Grades 3-4 (%) |

Infections and infestations

| | | |
|---|---|---|
| Infection (pathogen not specified)^{a} | 53 | 16 |
| Viral infection^{b} | 19 | 4 |
| Bacterial infection^{c} | 16 | 4 |

General disorders and administration site conditions

| | | |
|---|---|---|
| Asthenia^{d} | 46 | 4 |
| Edema^{e} | 27 | 1 |
| Pyrexia | 18 | 1 |

Gastrointestinal

| | | |
|---|---|---|
| Nausea^{f} | 42 | 4 |
| Diarrhea | 35 | 5 |
| Abdominal pain^{g} | 22 | 1 |
| Dysphagia | 16 | 0 |

Respiratory, thoracic and mediastinal

| | | |
|---|---|---|
| Dyspnea^{h} | 33 | 5 |
| Coughⁱ | 30 | 0 |
| Nasal congestion | 12 | 0 |

Vascular

| | | |
|---|---|---|
| Hemorrhage^{j} | 23 | 5 |
| Hypertension | 21 | 7 |

Musculoskeletal and connective tissue

| | | |
|---|---|---|
| Musculoskeletal pain^{k} | 22 | 4 |
| Muscle spasm | 17 | 0 |
| Arthralgia | 15 | 2 |

Nervous system

| | | |
|---|---|---|
| Headache^{l} | 21 | 0 |

Metabolism and nutrition

| | | |
|---|---|---|
| Decreased appetite | 17 | 1 |

Skin and subcutaneous

| | | |
|---|---|---|
| Rash^{m} | 12 | 0 |
| Pruritusⁿ | 11 | 0 |

^{a}infection with an unspecified pathogen includes acute sinusitis, device related infection, ear infection, folliculitis, gastroenteritis, gastrointestinal infection, hordeolum, infectious colitis, lung infection, skin infection, tooth infection, urinary tract infection, wound infection, upper respiratory tract infection, pneumonia, conjunctivitis, sinusitis, respiratory tract infection, bronchitis, sepsis, septic shock.
^{b}includes influenza, rhinovirus infection, gastroenteritis viral, viral upper respiratory tract infection, bronchitis viral, Epstein-Barr viremia, Epstein-Barr virus infection, parainfluenzae virus infection, Varicella zoster virus infection, viral infection.
^{c}includes cellulitis, Helicobacter infection, Staphylococcal bacteremia, catheter site cellulitis, Clostridium difficile colitis, Escherichia urinary tract infection, gastroenteritis Escherichia coli, Pseudomonas infection, urinary tract infection bacterial.
^{d}includes fatigue, asthenia, malaise.
^{e}includes edema peripheral, generalized edema, face edema, localized edema, edema.
^{f}includes nausea, vomiting.
^{g}includes abdominal pain, abdominal pain upper, abdominal pain lower.
^{h}includes dyspnea, dyspnea exertional, apnea, orthopnea, sleep apnea syndrome.
ⁱincludes cough, productive cough.
^{j}includes contusion, hematoma, epistaxis, increased tendency to bruise, conjunctival hemorrhage, hematochezia, mouth hemorrhage, catheter site hemorrhage, hematuria, hemothorax, purpura.
^{k}includes pain in extremity, back pain, flank pain, limb discomfort, musculoskeletal chest pain, neck pain, musculoskeletal pain.
^{l}includes headache, migraine.
^{m}includes rash, rash maculo-papular, rash erythematous, rash generalized, dermatitis exfoliative.
ⁿincludes pruritus, pruritus generalized.

Table 36 summarizes the laboratory abnormalities in belumosudil. ^{a} infection with an unspecified pathogen includes acute sinusitis, device related infection, ear infection, folliculitis, gastroenteritis, gastrointestinal infection, hordeolum, infectious colitis, lung infection, skin infection, tooth infection, urinary tract infection, wound infection, upper respiratory tract infection, pneumonia, conjunctivitis, sinusitis, respiratory tract infection, bronchitis, sepsis, septic shock.^{b} includes influenza, rhinovirus infection, gastroenteritis viral, viral upper respiratory tract infection, bronchitis viral, Epstein-Barr viremia, Epstein-Barr virus infection, parainfluenzae virus infection, Varicella zoster virus infection, viral infection.^{c} includes cellulitis, Helicobacter infection, Staphylococcal bacteremia, catheter site cellulitis, Clostridium difficile colitis, Escherichia urinary tract infection, gastroenteritis Escherichia coli, Pseudomonas infection, urinary tract infection bacterial.^{d} includes fatigue, asthenia, malaise.^{e} includes edema peripheral, generalized edema, face edema, localized edema, edema.^{f} includes nausea, vomiting.^{g} includes abdominal pain, abdominal pain upper, abdominal pain lower.^{h} includes dyspnea, dyspnea exertional, apnea, orthopnea, sleep apnea syndrome.ⁱ includes cough, productive cough.^{j} includes contusion, hematoma, epistaxis, increased tendency to bruise, conjunctival hemorrhage, hematochezia, mouth hemorrhage, catheter site hemorrhage, hematuria, hemothorax, purpura.^{k} includes pain in extremity, back pain, flank pain, limb discomfort, musculoskeletal chest pain, neck pain, musculoskeletal pain.^{l} includes headache, migraine.^{m} includes rash, rash maculo-papular, rash erythematous, rash generalized, dermatitis exfoliative.ⁿ includes pruritus, pruritus generalized.

TABLE 36
Selected Laboratory Abnormalities in Patients with
Chronic GVHD Treated with Belumosudil.

| | Belumosudil | | |
|---|---|---|---|
| | 200 mg once daily | | |
| | Grade 0-1 | Grade 2-4 | Grade 3-4 |
| | Baseline | Max Post | Max Post |
| Parameter | (N) | (%) | (%) |
| Chemistry | | | |
| Phosphate Decreased | 76 | 28 | 7 |
| Gamma Glutamyl | 47 | 21 | 11 |
| Transferase Increased | | | |
| Calcium Decreased | 82 | 12 | 1 |
| Alkaline Phosphatase Increased | 80 | 9 | 0 |
| Potassium Increased | 82 | 7 | 1 |
| Alanine Aminotransferase | 83 | 7 | 2 |
| Increased | | | |
| Creatinine Increased | 83 | 4 | 0 |
| Hematology | | | |
| Lymphocytes Decreased | 62 | 29 | 13 |
| Hemoglobin Decreased | 79 | 11 | 1 |
| Platelets Decreased | 82 | 10 | 5 |
| Neutrophil Count Decreased | 83 | 8 | 4 |

### Drug Drug Interactions

### Effect of Other Drugs on Belumosudil

Strong CYP3A Inducers: Coadministration of belumosudil with strong CYP3A inducers decreases belumosudil exposure, which may reduce the efficacy of belumosudil. Increase the dosage of belumosudil when coadministered with strong CYP3A inducers.

Proton Pump Inhibitors: Coadministration of belumosudil with proton pump inhibitors decreases belumosudil exposure, which may reduce the efficacy of belumosudil. Increase the dosage of beludosumil when coadministered with proton pump inhibitors.

### Pediatric Use

The safety and effectiveness of belumosudil have been established in pediatric patients 12 years and older. Use of belumosudil in this age group is supported by evidence from adequate and well-controlled studies of belumosudil in adults with additional population pharmacokinetic data demonstrating that age and body weight had no clinically meaningful effect on the pharmacokinetics of drug substance, that the exposure of drug substance is expected to be similar between adults and pediatric patients age 12 years and older, and that the course of disease is sufficiently similar in adult and pediatric patients to allow extrapolation of data in adults to pediatric patients.

### Geriatric Use

Of the 186 patients with chronic GVHD in clinical studies of belumosudil, 26% were 65 years and older. No clinically meaningful differences in safety or effectiveness of belumosudil were observed in comparison to younger patients.

### Pharmacokinetics

The following pharmacokinetic parameters are presented for chronic GVHD patients administered belumosudil 200 mg once daily, unless otherwise specified. The mean (% coefficient of variation, % CV) steady-state AUC and Cmax of belumosudil was 22700 (48%) hong/mL and 2390 (44%) ng/ml, respectively. Belumosudil Cmax and AUC increased in an approximately proportional manner over a dosage range of 200 and 400 mg (1 to 2 times once daily recommended dosage). The accumulation ratio of belumosudil was 1.4.

Absorption: Median Tmax of belumosudil at steady state was 1.26 to 2.53 hours following administration of 200 mg once daily or twice daily in patients. The mean (% CV) bioavailability was 64% (17%) following a single belumosudil dose in healthy subjects.

Effect of Food. Belumosudil Cmax and AUC increased 2.2 times and 2 times, respectively, following administration of a single belumosudil dose with a high-fat and high-calorie meal (800 to 1,000 calories with approximately 50% of total caloric content of the meal from fat) compared to the fasted state in healthy subjects. Median Tmax was delayed 0.5 hours.

Distribution: The geometric mean volume of distribution after a single dose of belumosudil in healthy subjects was 184 L (geo CV % 67.7%).

Belumosudil binding to human serum albumin and human al-acid glycoprotein was 99.9% and 98.6%, respectively, in vitro.

Elimination: The mean (% CV) elimination half-life of belumosudil was 19 hours (39%), and clearance was 9.83 L/hours (46%) in patients.

Metabolism: Belumosudil is primarily metabolized by CYP3A4 and to a lesser extent by CYP2C8, CYP2D6, and UGT1A9, in vitro.

Excretion: Following a single oral dose of radiolabeled belumosudil in healthy subjects, 85% of radioactivity was recovered in feces (30% as unchanged) and less than 5% in urine.

Specific Populations: No clinically significant differences in belumosudil pharmacokinetics were observed with regard to age (18 to 77 years), sex, weight (38.6 to 143 kg), or mild to moderate renal impairment (eGFR≥60 and <90 mL/min/1.72 m² to eGFR≥30 and <60 mL/min/1.72 m²). The effect of severe renal impairment on the pharmacokinetics of belumosudil has not been studied.

### Drug Interaction Studies

Clinical Studies and Model-Informed Approaches Effects of Other Drugs on Belumosudil

Strong Cytochrome P450 (CYP) 3A Inhibitors: There was no clinically meaningful effect on belumosudil exposure when coadministered with itraconazole in healthy subjects.

Strong CYP3A Inducers: Coadministration of rifampin decreased belumosudil Cmax by 59% and AUC by 72% in healthy subjects.

Moderate CYP3A Inducers: Coadministration of efavirenz is predicted to decrease belumosudil Cmax by 32% and AUC by 35% in healthy subjects.

Proton Pump Inhibitors: Coadministration of rabeprazole decreased belumosudil Cmax by 87% and AUC by 80%, and omeprazole decreased belumosudil Cmax by 68% and AUC by 47% in healthy subjects.

### Effects of Belumosudil on Other Drugs

CYP3A Substrates: Coadministration of belumosudil is predicted to increase midazolam (a sensitive CYP3A substrate) Cmax and AUC approximately 1.3- and 1.5-fold, respectively.

CYP2C9 Substrates: Coadministration of belumosudil is not expected to have clinically meaningful effect on the exposure of CYP2C9 substrates (such as warfarin).

CYP2C8 Substrates: Coadministration of belumosudil is not expected to have clinically meaningful effect on the exposure of CYP2C8 substrates that are not an OATPIB1 substrate.

### In Vitro Studies

Transporter Systems: Belumosudil is a substrate of P-gp. Belumosudil inhibits BCRP, P-gp, and OATPIB1 at clinically relevant concentrations.

Enzymes Systems: Belumosudil is an inhibitor of CYP1A2, CYP2C19, CYP2D6, UGT1A1 and UGT1A9.

Although the present invention has been described in some detail by way of illustration and example for purposes of clarity and understanding, the descriptions and examples should not be construed as limiting the scope of the invention. The disclosures of all patent and scientific literature cited herein are expressly incorporated herein in their entirety by reference.

1-21. (canceled)

22. A method of treating cGVHD in a subject receiving ruxolitinib as a second line treatment, comprising ceasing or instructing the cessation of the administration of ruxolitinib as a second line treatment, and administering to a patient in need thereof a therapeutically effective amount of 2-{3-[4-(1H-indazol-5-ylamino)-2-quinazolinyl]phenoxy}-N-(propan-2-yl) acetamide or a pharmaceutically acceptable salt thereof, as a third line treatment.

23. The method of claim 22, wherein the 2-{3-[4-(1H-indazol-5-ylamino)-2-quinazolinyl]phenoxy}-N-(propan-2-yl) acetamide or a pharmaceutically acceptable salt thereof comprises 2-{3-[4-(1H-indazol-5-ylamino)-2-quinazolinyl]phenoxy}-N-(propan-2-yl) acetamide methanesulfonate (1:1).

24. The method of claim 22, wherein the daily dose of 2-{3-[4-(1H-indazol-5-ylamino)-2-quinazolinyl]phenoxy}-N-(propan-2-yl) acetamide or a pharmaceutically acceptable salt thereof administered to the subject is in the range of a dose equivalent to about 200-400 mg of the free base of 2-{3-[4-(1H-indazol-5-ylamino)-2-quinazolinyl]phenoxy}-N-(propan-2-yl) acetamide.

25. The method of claim 22, wherein the subject is receiving concomitant corticosteroid therapy.

26. The method of claim 25, wherein the dose of the concomitant corticosteroid therapy is reduced after at least 1 cycle of the treatment with 2-{3-[4-(1H-indazol-5-ylamino)-2-quinazolinyl]phenoxy}-N-(propan-2-yl) acetamide or a pharmaceutically acceptable salt thereof.

27. The method of claim 25, wherein the concomitant corticosteroid therapy is discontinued after at least 1 cycle of the treatment with 2-{3-[4-(1H-indazol-5-ylamino)-2-quinazolinyl]phenoxy}-N-(propan-2-yl) acetamide or a pharmaceutically acceptable salt thereof.

28. A method of treating cGVHD in a subject receiving ruxolitinib, the method comprising the steps of:
(a) confirming the subject is refractory to treatment with ruxolitinib;
(b) ceasing or instructing the cessation of administration of ruxolitinib to the subject when the cGVHD is not responding to treatment with ruxolitinib; and
(c) administering a therapeutically effective amount of 2-{3-[4-(1H-indazol-5-ylamino)-2-quinazolinyl]phenoxy}-N-(propan-2-yl) acetamide or a pharmaceutically acceptable salt thereof, to the subject in need thereof.

29. The method of claim 28, wherein the ruxolitinib is administered as a second line treatment for the cGVHD and/or wherein the 2-{3-[4-(1H-indazol-5-ylamino)-2-quinazolinyl]phenoxy}-N-(propan-2-yl) acetamide or a pharmaceutically acceptable salt thereof is administered as a third line treatment for the cGVHD.

30. The method of claim 28, wherein the 2-{3-[4-(1H-indazol-5-ylamino)-2-quinazolinyl]phenoxy}-N-(propan-2-yl) acetamide or a pharmaceutically acceptable salt thereof comprises 2-{3-[4-(1H-indazol-5-ylamino)-2-quinazolinyl]phenoxy}-N-(propan-2-yl) acetamide methanesulfonate (1:1).

31. The method of claim 28, wherein the daily dose of 2-{3-[4-(1H-indazol-5-ylamino)-2-quinazolinyl]phenoxy}-N-(propan-2-yl) acetamide or a pharmaceutically acceptable salt thereof administered to the subject is in the range of about 200 mg to about 400 mg.

32. The method of claim 28, wherein the dose of 2-{3-[4-(1H-indazol-5-ylamino)-2-quinazolinyl]phenoxy}-N-(propan-2-yl) acetamide or a pharmaceutically acceptable salt thereof for the subject is about 200 mg taken once daily with food.

33. The method of claim 28, wherein the daily dose of 2-{3-[4-(1H-indazol-5-ylamino)-2-quinazolinyl]phenoxy}-N-(propan-2-yl) acetamide or a pharmaceutically acceptable salt thereof for the subject is in the range of a dose equivalent to about 200-400 mg of the free base of 2-{3-[4-(1H-indazol-5-ylamino)-2-quinazolinyl]phenoxy}-N-(propan-2-yl) acetamide.

34. The method of claim 28, wherein the subject has failed one to three prior lines of systemic therapy for the cGVHD

35. The method of claim 28, wherein the subject has failed at least two prior lines of systemic therapy for the cGVHD.

36. A method of treating cGVHD in a subject receiving a steroid as a first line treatment for the cGVHD and a first rho-associated coiled coil-containing protein kinase-2 (ROCK2) inhibitor as a second line treatment for the cGVHD, comprising ceasing or instructing the cessation of the administration of the first ROCK2 inhibitor as a second line treatment, and administering in the patient in need thereof a therapeutically effective amount of a second ROCK2 inhibitor as a third line treatment.

37. The method of claim 36, wherein the first ROCK2 inhibitor comprises ruxolitinib.

38. The method of claim 36, wherein the second ROCK2 inhibitor comprises 2-{3-[4-(1H-indazol-5-ylamino)-2-quinazolinyl]phenoxy}-N-(propan-2-yl) acetamide or a pharmaceutically acceptable salt thereof.

39. The method of claim 38, wherein the daily dose of the second ROCK2 inhibitor administered to the subject is in the range of a dose equivalent to about 200-400 mg of the free base of 2-{3-[4-(1H-indazol-5-ylamino)-2-quinazolinyl]phenoxy}-N-(propan-2-yl) acetamide.

40. A method of treating chronic graft-versus-host disease (cGVHD) in a subject after failure of at least two prior lines of systemic therapy, the method comprising administering to the patient in need thereof a therapeutically effective amount of 2-{3-[4-(1H-indazol-5-ylamino)-2-quinazolinyl]phenoxy}-N-(propan-2-yl) acetamide, or a pharmaceutically acceptable salt thereof, wherein:
one of the at least two prior lines of systemic therapy comprises treatment with a compound that inhibits rho-associated coiled coil-containing protein kinase-2 (ROCK2);
the compound that inhibits ROCK2 is ruxolitinib; and
the therapeutically effective amount of 2-{3-[4-(1H-indazol-5-ylamino)-2-quinazolinyl]phenoxy}-N-(propan-2-yl) acetamide or a pharmaceutically acceptable salt thereof, is a daily dose that is equivalent to about 200 mg of the free base of 2-{3-[4-(1H-indazol-5-ylamino)-2-quinazolinyl]phenoxy}-N-(propan-2-yl) acetamide.

41. The method of claim 40, wherein the subject is refractory to prior treatment with ruxolitinib.
